(19) United States
(12) Patent Application Publication
(10) Pub. No.: US 2023/0167467 A1
(43) Pub. Date: Jun. 1, 2023

(54) METHODS AND COMPOSITIONS FOR THE PRODUCTION OF ISOBUTENE

(71) Applicant: University of Alaska Anchorage, Anchorage, AK (US)

(72) Inventor: Brandon Briggs, Anchorage, AK (US)

(21) Appl. No.: 17/919,081

(22) PCT Filed: Apr. 15, 2021
(86) PCT No.: PCT/US2021/027510

§ 371 Date: Oct. 14, 2022

Related U.S. Application Data
(60) Provisional application No. 63/010,409, filed on Apr. 15, 2020.

(51) Int. Cl. C12P 5/02; C12N 9/12; C12N 9/88; C12N 15/70; C12N 15/90

(52) U.S. Cl. CPC C12P 5/026; C12N 9/1205; C12Y 207/01036; C12N 9/88; C12Y 401/01033; C12N 15/70; C12N 15/90; C12N 2800/101

(57) ABSTRACT

Disclosed are nucleic acid sequences comprising a first E. coli homology region, wherein the first E. coli homology region comprises a protospacer adjacent motif (PAM) mutation; a constitutive promoter; a mevalonate-3-kinase (M3K) gene; a mevalonate diphosphate decarboxylase (MVD) gene; and a second E. coli homology region. Disclosed are vectors comprising one or more of the disclosed nucleic acid sequences. Disclosed are recombinant cells comprising a nucleic acid sequence, wherein the nucleic acid sequence comprises a first E. coli homology region, wherein the first E. coli homology region comprises a PAM mutation; a constitutive promoter; a M3K gene; a MVD gene; and a second E. coli homology region.

### CROSS-REFERENCE TO RELATED APPLICATIONS

This application claims the benefit of U.S. Provisional Patent Application No. 63/010,409, filed on Apr. 15, 2020, which is incorporated by reference herein in its entirety.

### BACKGROUND

Isobutene is a key precursor for numerous chemicals and products. For example, isobutene is used to produce butyl rubber, terephthalic acid, and a gasoline performance additive (alkylate). Alkylate increases octane, improves combustion, reduces emissions and prevents engine knock. Unfortunately, the production of these olefins requires high-energy reactions (steam cracking) from petroleum sources. More efficient reactions that produce industrially significant hydrocarbons are needed.

Biological production of isobutene has been known for many years. A variety of eukaryotes, archaea, bacteria, fungi, and plants naturally produce these compounds in low concentrations through several identified pathways. Microbial processes can produce isobutene that can be used for industrial applications given key advances in production. Furthermore, using microorganisms allows for less dependence on petroleum sources because diverse feedstocks can be used such as corn stover, wastewater, or manure.

One particular microbial pathway that produces isobutene is the mevalonate (MVA) pathway. The MVA pathway is the main route for the production of isopentenyl pyrophosphate, a key building block for a large family of biological metabolites. The last enzyme in this pathway is mevalonate diphosphate decarboxylase (MVD) and MVD has the ability to decarboxylate 3-hydroxyisovalerate (3-HIV) to isobutene (Reaction 1). An enzyme in the MVA pathway of Picrophilus torridus (Archaea from acidic environments) has been identified as a mevalonate-3-kinase (M3K). This enzyme has the highest rate of isobutene formation by catalyzing the phosphorylation of 3-HIV into an unstable 3-phosphate intermediate that undergoes spontaneous decarboxylation to form isobutene (Reaction 2).

C₅H₉O₃+ATP→C₄H₈+CO₂+Pi (Reaction 1)

C₅H₉O₃+ATP→C₅H₈O₃P→C₄H₈+CO₂+Pi (Reaction 2)

Both the MVD and M3K genes have been independently engineered into Escherichia colt using plasmid based systems. In synthetic media, isobutene production rates reached up to 507 pmol min⁻¹ g cells⁻¹ when M3K was expressed in E. coli.

Despite the advances in bioengineering, biological production of isobutene is still limited. In part, this is because of inefficient expression systems that require antibiotics (plasmid based) to be maintained or control of expression is limited. Another reason for the limited bio-production is because of unknown competing metabolic pathways. Microbial metabolism can be described as a complex network where the product of one reaction can be shuttled to several different competing reactions. For efficient bio-production competing reactions need to be minimized.

To overcome these obstacles, the current disclosure describes both biosynthetic genes for isobutene (M3K and MVD) inserted into the E. coli chromosome. In addition, expression levels can be increased by placing the genes under the control of the 16S rRNA gene promoter and by placing the genes in a part of the genome that is known to have higher expression levels. Placing the genes in the chromosome also removed the need for antibiotics to maintain the genes in a plasmid. Furthermore, these genes can work with native pathways to produce isobutene from simple organic sugars such as glucose or complex carbon such as manure. The isobutene production levels described herein have been pushed to 304 μmol L⁻¹ hr⁻¹.

### BRIEF SUMMARY

Disclosed are nucleic acid sequences comprising a first E. coli homology region, wherein the first E. coli homology region comprises a protospacer adjacent motif (PAM) mutation; a constitutive promoter; a mevalonate-3-kinase (M3K) gene; a mevalonate diphosphate decarboxylase (MVD) gene; and a second E. coli homology region.

Disclosed are vectors comprising one or more of the disclosed nucleic acid sequences.

Disclosed are recombinant cells comprising a nucleic acid sequence, wherein the nucleic acid sequence comprises a first E. coli homology region, wherein the first E. coli homology region comprises a PAM mutation; a constitutive promoter; a M3K gene; a MVD gene; and a second E. coli homology region.

Disclosed are methods of making recombinant cells comprising administering any one of the disclosed linear nucleic acid sequences to a cell, wherein the cell incorporates the linear nucleic acid sequence into the cellular genome. In some aspects the recombinant cells are bacterial cells.

Disclosed are methods of producing isobutene comprising culturing any one of the disclosed recombinant bacteria cells comprising a nucleic acid sequence comprising a constitutive promoter; a M3K gene and a MVD gene under conditions suitable for bacterial growth and expression of M3K and MVD, wherein the MVD decarboxylates 3-hydroxyisovalerate (3-HIV) to isobutene, and wherein the M3K catalyzes the phosphorylation of 3-HIV into an unstable 3-phosphate intermediate that undergoes spontaneous decarboxylation to isobutene.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a diagram of MVA pathway derivatives for production of industrial petrochemical isobutene.

FIG. 2 is a schematic of a nucleic acid sequence comprising M3K and MVD being inserted into an E. coli genome.

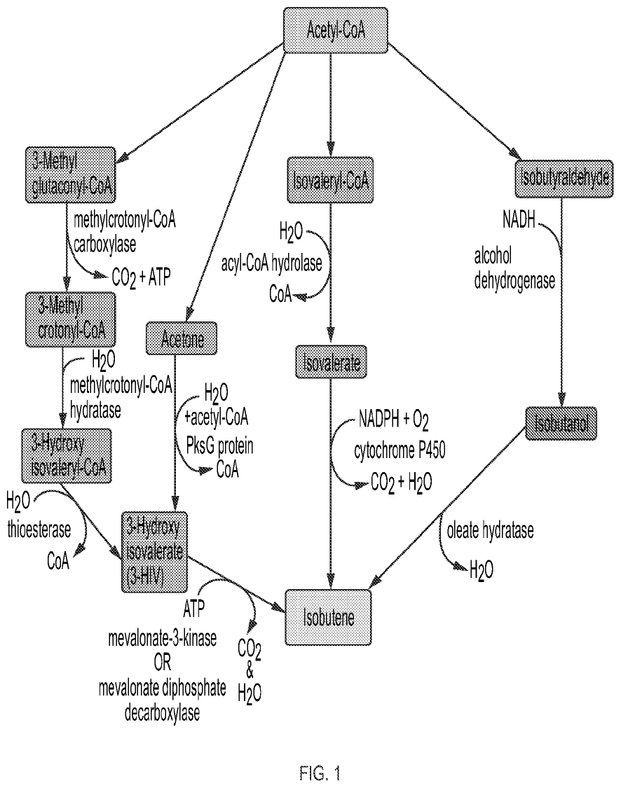

FIG. 1 shows solvent-induced dysregulation. Total (black bars) and relative (red and blue bars) number of dysregulated genes following solvent exposure. All data are significantly dysregulated genes with Bon Ferroni corrected p-value<0.05 when compared to no solvent control.

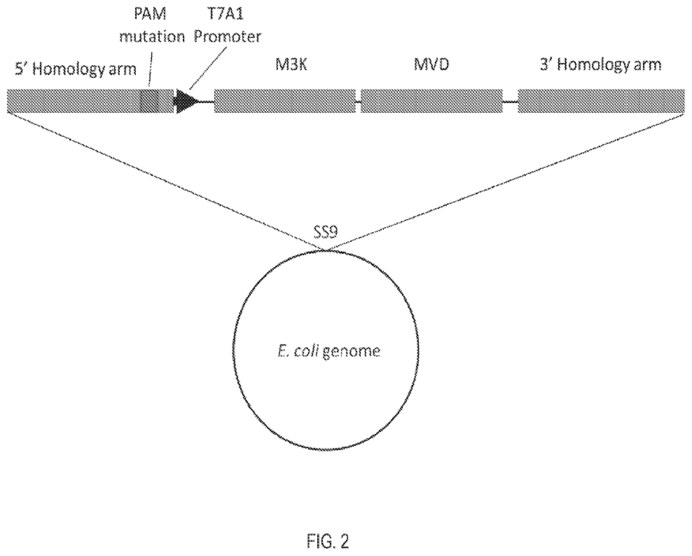

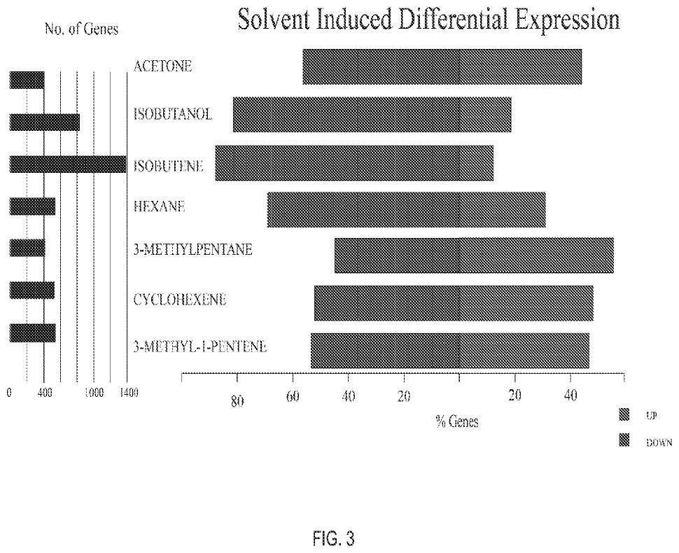

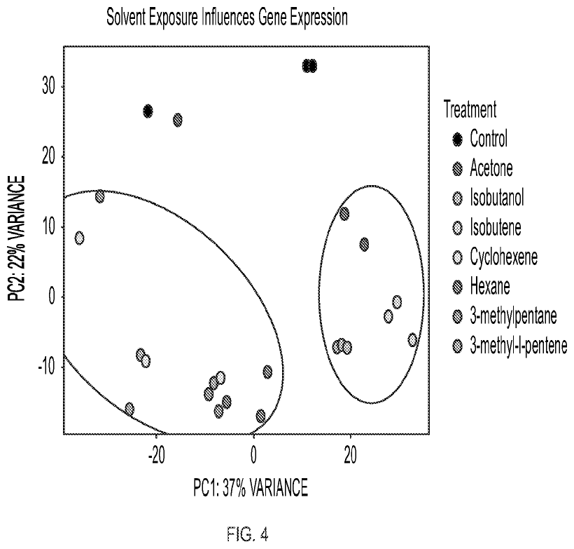

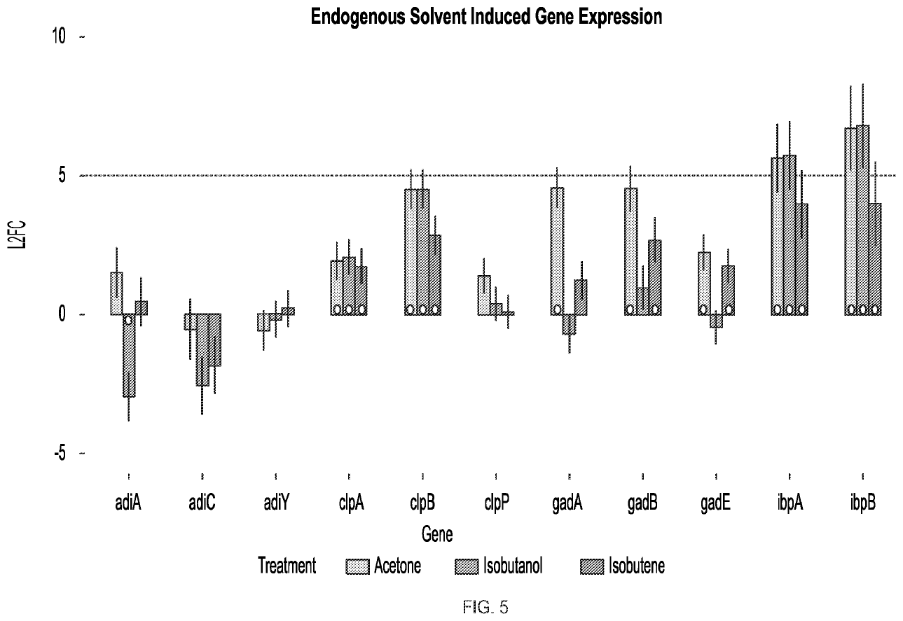

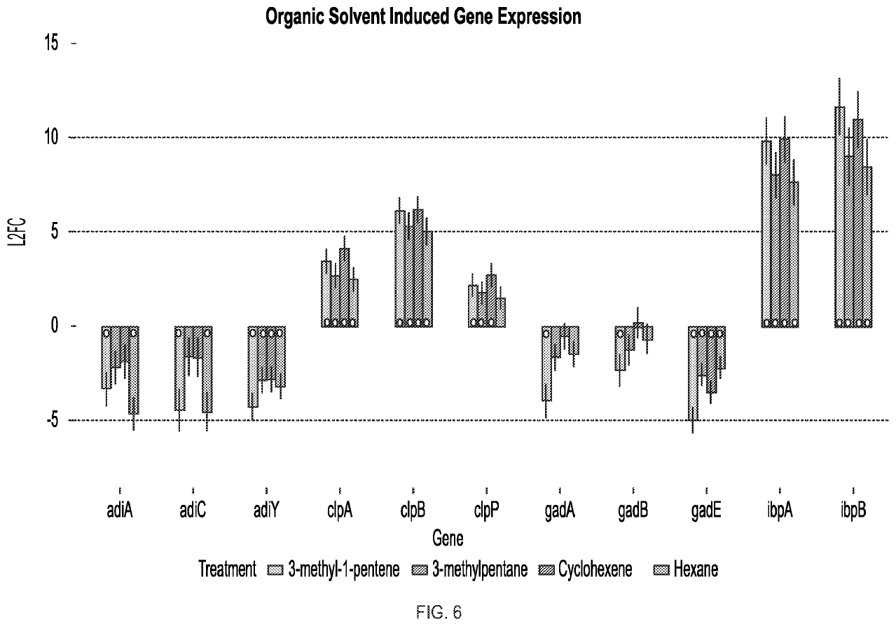

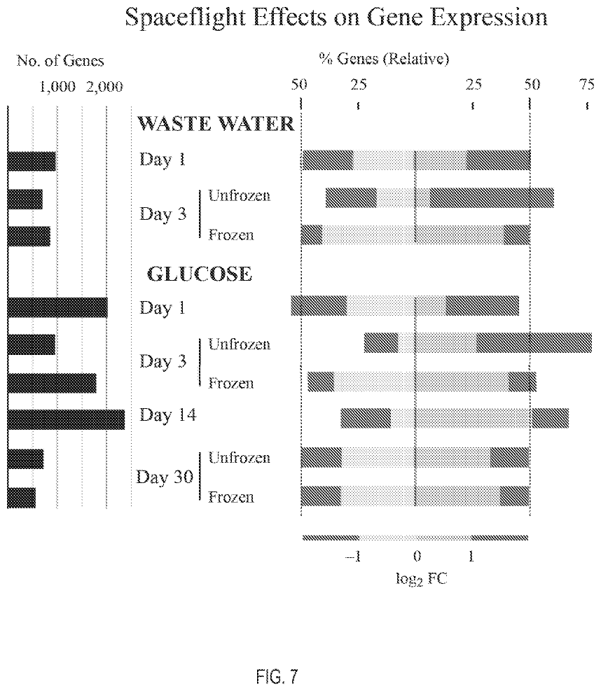

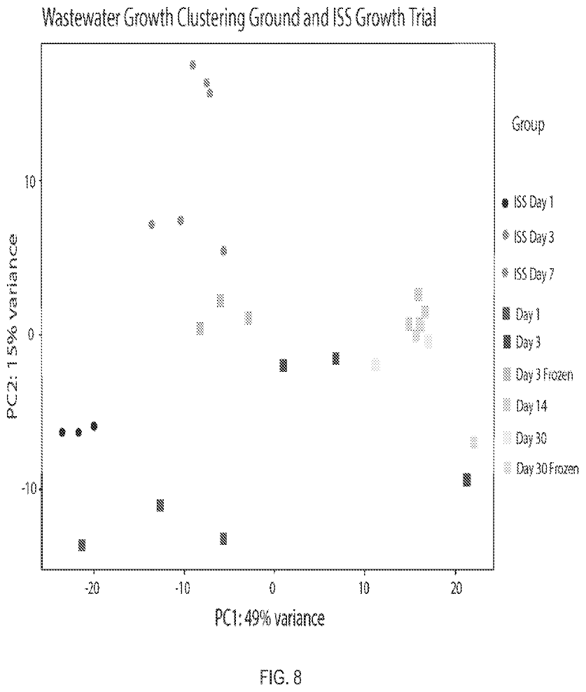

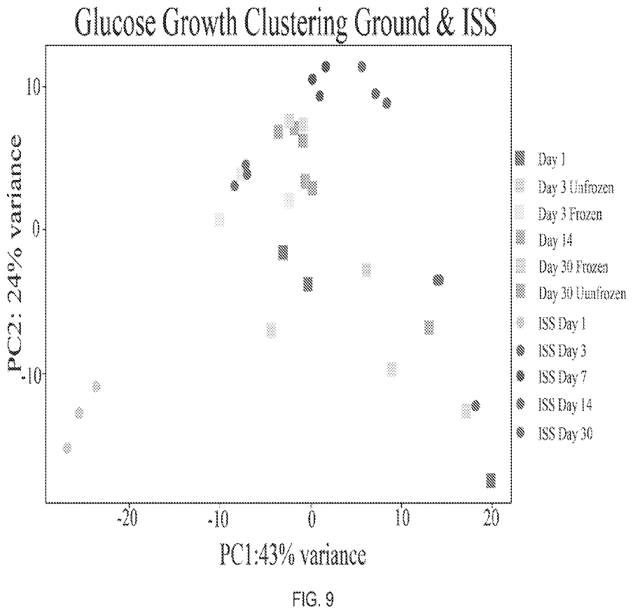

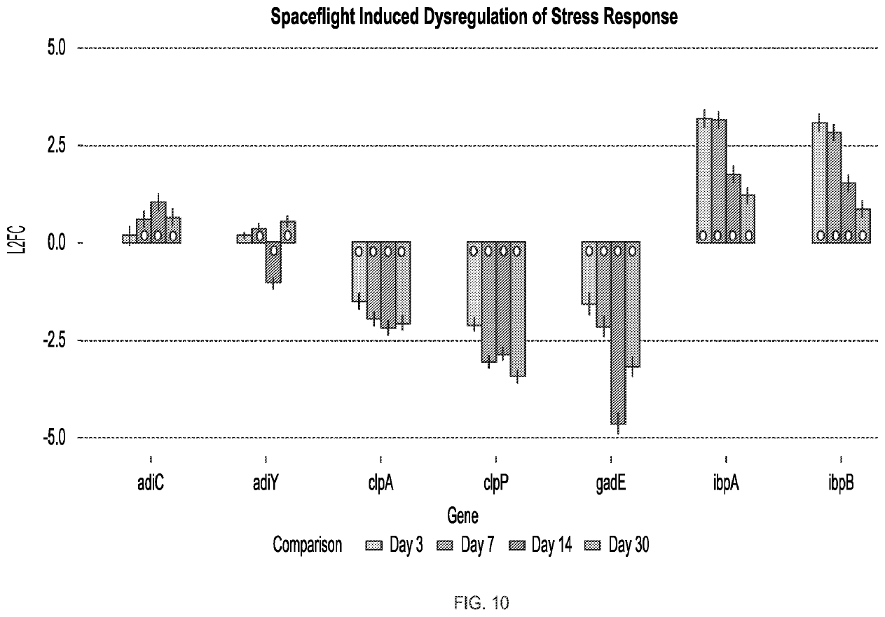

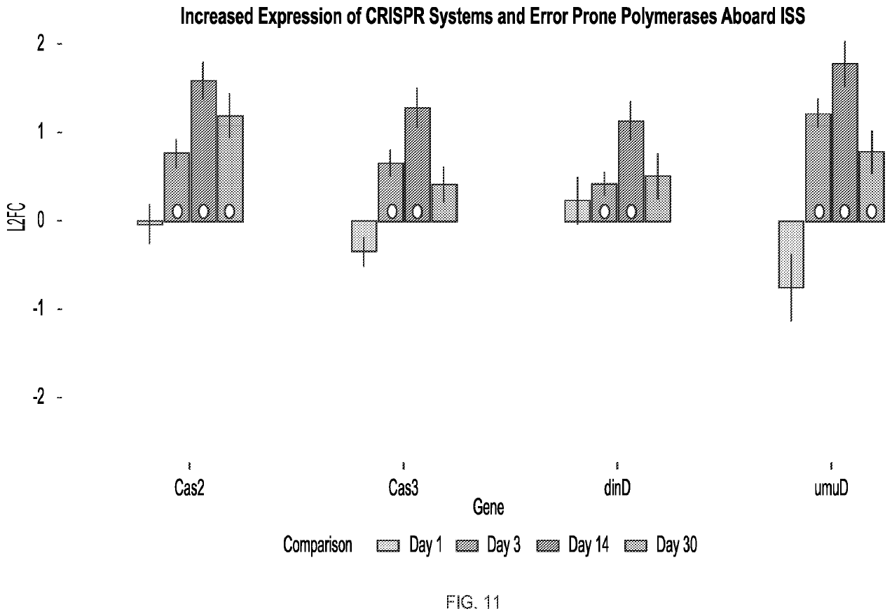

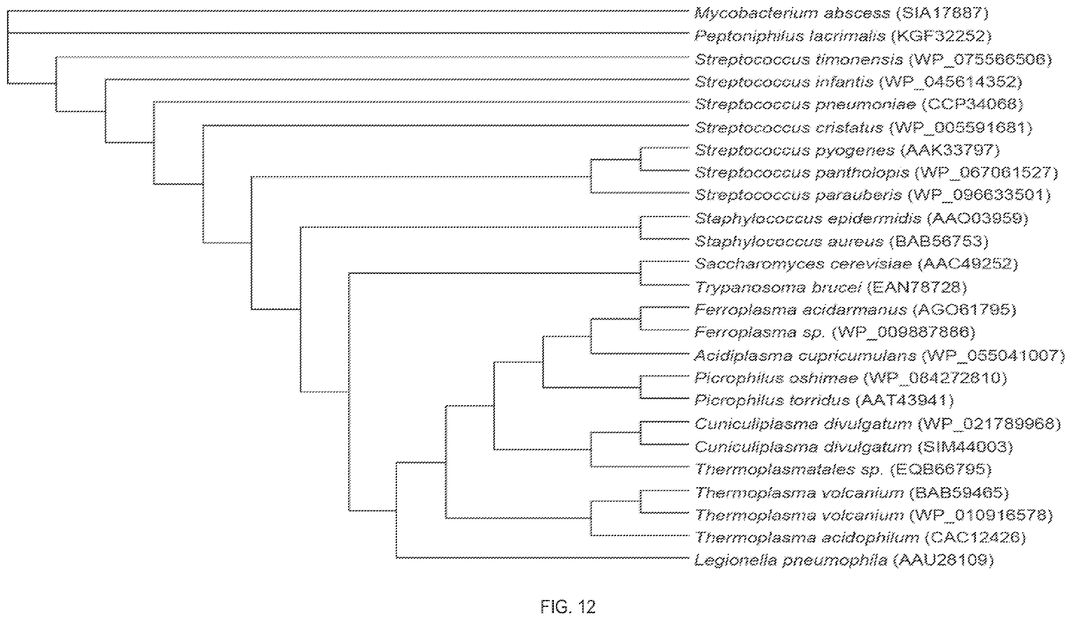

FIG. 2 shows solvent exposure clustering. Principle components analysis of effects of solvent exposure on gene expression. Components describe the variance in gene expression within the likelihood estimates generated by DESeq-2. Samples that cluster more closely showed more similar gene dysregulation responses. Treatments one and two cluster independently as demarcated by ovals surrounding treatment groups

FIG. 3 shows conserved responses to solvent exposure. Log₂Fold-Change of stress response genes undergoing changes in expression following exposure to acetone, isobutanol, and isobutene. Upregulation of chaperones clp and ibp gene families in all treatments. Mixed expression of acid stress responses adi and gad gene families. P_{A} <0.05 ∘. Error bars are ±log fold change standard error (LFCSE).

FIG. 4 shows conserved responses to hydrocarbon exposure. Log₂Fold-Change of stress response genes undergoing changes in expression following exposure to organic solvents. Conserved responses across all treatments were: upregulation of chaperones clp and ibp gene families and downregulation of acid stress responses adi and gad gene families. Padⱼ<0.05 ∘. Error bars are ±LFCSE.

FIG. 5 shows spaceflight effects on gene expression. Spaceflight induced differentially expressed gene counts, comparing equivalent time points of ground and ISS samples to screen for spaceflight induced effects. Black bars are total genes and colored bars are relative percentage of dysregulated genes. All data are significantly dysregulated genes with Bon Ferroni corrected p-value<0.05 when compared to samples grown on ground

FIG. 6 shows wastewater growth clustering ground and ISS growth trial. Principal components analysis plot showing clustering of changes in gene expression at different time points for ground (square) and ISS (circle) samples grown on MOPS+1% wastewater. Components describe the variance in gene expression within the likelihood estimates generated by DESeq-2. Samples that cluster more closely showed more similar gene dysregulation responses.

FIG. 7 shows glucose growth clustering ground and ISS. Principle components analysis plot showing clustering of changes in gene expression at different time points for ground (square) and ISS (circle) samples grown on MOPS+0.5% glucose. Components describe the variance in gene expression within the likelihood estimates generated by DESeq-2. Samples that cluster more closely showed more similar gene dysregulation responses

FIG. 8 shows spaceflight induced dysregulation of stress response. Changing expression of acid stress response, and protein repair systems over time aboard ISS. P_{adj} <0.05 ∘. Error bars are ±LFCSE.

FIG. 9 shows increased expression of CRISPR systems and error prone polymerases aboard ISS. As cultures age, they increase expression of chromosome protection systems for 14 days, expression has decreased by 30 days. P_{adj}<0.05 0. Error bars are ±LFCSE.

FIG. 12 shows a phylogenetic tree of all the M3K genes found using a hidden Markov model that was trained on already known M3K enzymes.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a nucleic acid sequence is disclosed and discussed and a number of modifications that can be made to a number of molecules including the nucleic acid sequence are discussed, each and every combination and permutation of the nucleic acid sequence and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a nucleic acid sequence” includes a plurality of such nucleic acid sequences, reference to “the MVD gene” is a reference to one or more MVD genes and equivalents thereof known to those skilled in the art, and so forth.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used herein the terms “amino acid” and “amino acid identity” refers to one of the 20 naturally occurring amino acids or any non-natural analogues that may be in any of the antibodies, variants, or fragments disclosed. Thus, “amino acid” as used herein means both naturally occurring and synthetic amino acids. For example, homophenylalanine, citrulline and norleucine are considered amino acids for the purposes of the invention. “Amino acid” also includes amino acid residues such as proline and hydroxyproline. The side chain may be in either the (R) or the (S) configuration. In some aspects, the amino acids are in the (S) or L-configuration. If non-naturally occurring side chains are used, non-amino acid substituents may be used, for example to prevent or retard in vivo degradation.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA. Another example is the operable linkage of the MVD gene and the M3K gene wherein each gene is suitably positioned or oriented for transcription from the same promoter.

The term “percent homology” or “% homology” is used interchangeably herein with the term “percent (%) identity” and refers to the level of nucleic acid or amino acid sequence identity when aligned with a wild type sequence using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90, 95, 98% or more sequence identity to a given sequence, e.g., the coding sequence for anyone of the inventive polypeptides, as described herein. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet. See also, Altschul, et al., 1990 and Altschul, et al., 1997. Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62matrix. (See, e.g., Altschul, S. F., et al., Nucleic Acids Res.25:3389-3402, 1997.) A preferred alignment of selected sequences in order to determine“% identity” between two or more sequences, is performed using for example, the CLUSTAL-W program in Mac Vector version 13.0.7, operated with default parameters, including an open gap penalty of 10.0, an extended gap penalty of 0.1, and a BLOSUM 30 similarity matrix.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

“Inhibit,” “inhibiting” and “inhibition” mean to diminish or decrease an activity, level, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

The term “fragment” can refer to a portion (e.g., at least 5, 10, 25, 50, 100, 125, 150, 200, 250, 300, 350, 400 or 500, etc. amino acids or nucleic acids) of a protein or nucleic acid molecule that is substantially identical to a reference protein or nucleic acid and retains the biological activity of the reference. In some aspects, the fragment or portion retains at least 50%, 75%, 80%, 85%, 90%, 95% or 99% of the biological activity of the reference protein or nucleic acid described herein. Further, a fragment of a referenced peptide can be a continuous or contiguous portion of the referenced polypeptide (e.g., a fragment of a peptide that is ten amino acids long can be any 2-9 contiguous residues within that peptide).

A “variant” can mean a difference in some way from the reference sequence other than just a simple deletion of an N- and/or C-terminal amino acid residue or residues. Where the variant includes a substitution of an amino acid residue, the substitution can be considered conservative or non-conservative. Conservative substitutions are those within the following groups: Ser, Thr, and Cys; Leu, Ile, and Val; Glu and Asp; Lys and Arg; Phe, Tyr, and Trp; and Gln, Asn, Glu, Asp, and His. Variants can include at least one substitution and/or at least one addition, there may also be at least one deletion. Variants can also include one or more non-naturally occurring residues. For example, they may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH2(CH₂)nCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Nucleic Acid Sequences

Disclosed are nucleic acid sequences comprising a first bacterial homology region, wherein the first bacterial homology region comprises a protospacer adjacent motif (PAM) mutation; a constitutive promoter; a mevalonate-3-kinase (M3K) gene; a mevalonate diphosphate decarboxylase (MVD) gene; and a second bacterial homology region. In some aspects, the first or second bacterial homology regions can be homologous to the particular bacteria (e.g. subject bacteira) to which the nucleic acid sequences will be administered to. For example, if the disclosed nucleic acid sequences will be administered to or introduced to E. coli, then the homology regions can be E. coli homology regions.

Disclosed are nucleic acid sequences comprising a first E. coli homology region, wherein the first E. coli homology region comprises a protospacer adjacent motif (PAM) mutation; a constitutive promoter; a mevalonate-3-kinase (M3K) gene; a mevalonate diphosphate decarboxylase (MVD) gene; and a second E. coli homology region.

Disclosed are nucleic acid sequences comprising a first E. coli homology region, wherein the first E. coli homology region comprises a PAM mutation; a constitutive promoter; a M3K gene comprising the sequence of SEQ ID NO:1; a MVD gene comprising the sequence of SEQ ID NO:2; and a second E. coli homology region.

Disclosed are nucleic acid sequences comprising a first E. coli homology region, wherein the first E. coli homology region comprises a PAM mutation; a constitutive promoter; a M3K gene comprising a sequence having at least 90% identity to the sequence of SEQ ID NO:1; a MVD gene comprising a sequence having at least 90% identity to the sequence of SEQ ID NO:2; and a second E. coli homology region.

In some aspects, one or both of the M3K gene and the MVD gene are optimized.

In some aspects, one or both of the M3K gene and the MVD gene are optimized for E. coli. Nucleic acid sequences can be codon optimized in order to improve gene expression and or increase translational efficiency of a sequence of interest in a host organism. For example, M3K, derived from Picrophilus torridus, can be codon optimized for better expression in E. coli.

In some aspects, the MVD gene and the M3K gene are operably linked.

In some aspects, the nucleic acid sequence is linear. In some aspects, the nucleic acid sequence is circular.

In some aspects, the nucleic acid sequence comprises, from 5′ to 3′ respectively, a first E. coli homology region, wherein the first E. coli homology region comprises a PAM mutation; a constitutive promoter; a M3K gene; a MVD gene; and a second E. coli homology region.

In some aspects, variants of one or both of the M3K gene and the MVD gene can be used. Variants can include nucleotide sequences that are substantially similar to sequences of the M3K gene or the MVD gene, precursors or sequences derived thereof. In an aspect, variants include nucleotide sequences that are substantially similar to the of the M3K gene or the MVD gene sequence or fragments thereof. Variants can also include nucleotide sequences that are substantially similar to sequences of the M3K gene or the MVD gene disclosed herein. A “variant” can mean a difference in some way from the reference sequence other than just a simple deletion of an N- and/or C-terminal nucleotide. Variants can also or alternatively include at least one substitution and/or at least one addition, there may also be at least one deletion. In some aspects, the variant M3K gene or the variant MVD gene to be used can comprise a sequence displaying at least 80% sequence identity to the sequence of the M3K gene (SEQ ID NO: 1) or the MVD gene (SEQ ID NO: 2). In some aspects, the M3K gene or the MVD gene to be used can comprise a sequence displaying at least 90% sequence identity to SEQ ID NO: 1 or 2. In some aspects, the M3K gene or the MVD gene to be used can comprise a sequence displaying at least 91%, 92%, 93%, 94%, 95%, 96%, 97%, 98% or 99% sequence identity to SEQ ID NO: 1 or 2.

Alternatively or in addition, variants can comprise modifications, such as non-natural residues at one or more positions with respect to the M3K gene or the MVD gene sequence. In an aspect, the variant can be a sequence wherein the last nucleotide of the M3K gene or the MVD gene is changed. In some aspects, the variant can be a sequence comprising at least one, at least two, or at least three substitutions at the 5′ end of the M3K gene or the MVD gene. In an aspect, nucleotide substitutions can include nucleotide substitutions to the reference sequence which increase stability of the M3K gene or the MVD gene or a variant thereof. Nucleotide substitutions can be substitutions of one or two bases. In some aspects, nucleotide substitutions can be substitutions of three bases. Deletions and insertions can include from one (1) to about three (3) bases. .Substitutions, deletions, insertions or any combination thereof may be used to arrive at a final derivative or variant. Generally, these changes are done on a few nucleotides to minimize the alteration of the molecule. However, larger changes may be tolerated in certain circumstances.

Generally, the nucleotide identity between individual variant sequences can be at least 90%, 91%, 92%, 93%, 94%, 95%, 96%, 97%, 98%, 99%, or 100%. Thus, a “variant sequence” can be one with the specified identity to the parent or reference sequence of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. For example, a “variant sequence” can be a sequence that contains 1, 2, or 3 4 nucleotide base changes as compared to the parent or reference sequence of the invention, and shares or improves biological function, specificity and/or activity of the parent sequence. In some aspects, the parent or reference sequence can be miR-584-5p.

In some aspects, any of sequences disclosed herein can include a single nucleotide change as compared to the parent or reference sequence. In some aspects, any of the sequences disclosed herein can include at least two nucleotide changes as compared to the parent or reference sequence. The nucleotide identity between individual variant sequences can be at least 60%, 65%, 70%, 75%, 80%, 85%, 90%, 91%, 92%, 93%, 94%, 95%, 96%, 97%, 98%, 99% or 100%. Thus, a “variant sequence” can be one with the specified identity to the parent sequence of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. The variant sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence.

### 1. Bacterial Homology Region

Disclosed are nucleic acid sequences comprising a first bacterial homology region. Also disclosed are nucleic acid sequences comprising a second bacterial homology region. In some aspects, disclosed are nucleic acid sequences comprising a first and second bacterial homology region. The bacterial homology regions can be designed to allow the disclosed nucleic acid sequences comprising the bacterial homology regions to use homologous recombination to insert the sequences between the first and second bacterial homology regions into the bacterial chromosome.

Disclosed are nucleic acid sequences comprising a first E. coli homology region. Also disclosed are nucleic acid sequences comprising a second E. coli homology region. In some aspects, disclosed are nucleic acid sequences comprising a first and second E. coli homology region. The E. coli homology regions are designed to allow the disclosed nucleic acid sequences comprising the E. coli homology regions to use homologous recombination to insert the sequences between the first and second E. coli homology regions into the E. coli chromosome.

In some aspects, the first or second E. coli homology regions are homologous to the E. coli strain MG1655. In some aspects, the first or second E. coli homology regions are homologous to the E. coli strain DH5alpha. In some aspects, the first or second E. coli homology regions are homologous to E. coli strain K12.

In some aspects, the E. coli homology regions can be nucleic acid sequences homologous to E. coli safe site 9 sequences.

In some aspects, the first E. coli homology region comprises a PAM mutation. In some aspects, the PAM mutation is a mutation in the wild type sequence AAGG to the PAM mutation of CAAA. In some aspects, a PAM site is a site where a guide RNA directs a Cas protein allowing for a double stranded cut of the DNA. In some aspects, the presence of a PAM mutation in a bacterial homology region can result in the guide RNA and Cas protein not being able to cut the DNA. Thus, in some aspects, only DNA sequences that have an unmutated PAM site would be cut and those DNA sequences would be understood to have not undergone homologous recombination with a nucleic acid comprising the bacterial homology regions.

In some aspects, the bacterial homology regions can be reduced in size. For example, the first and second E. coli homology regions can be cut down from 500 bp to 400 bp (see ACS Synth. Biol. 2016, 5, 7, 561-568, hereby incorporated by reference herein).

### 2. Promoters

In some aspects, any known constitutive promoter or regulatable promoter can be used in the disclosed nucleic acid sequences.

In some aspects, the constitutive promoter is a 16S rRNA promoter. In some aspects, the constitutive promoter is a T7A1 promoter.

In some aspects, the constitutive promoter is located 3′ of the first E. coli homology region and 5′ of the M3K gene.

In some aspects, any promoter compatible with bacterial expression systems can be used.

3. M3K gene

M3K, a key enzyme in the MVA pathway, is an ATP-dependent enzyme that catalyzes the conversion of 3-hydroxyisovalerate (3-HIV) to isobutene by catalyzing phosphorylation of 3-HIV into an unstable 3-phosphate intermediate that undergoes spontaneous decarboxylation to form isobutene.

In some aspects, an optimized M3K gene can comprise the following nucleic acid sequence

(SEQ ID NO: 1)
ATGGAGAACTATAATGTTAAAACCCGTGCATTTCC
GACCATTGGTATTATTCTGCTGGGTGGCATTAGCG
ACAAAAAAAACCGTATTCCGCTGCATACCACCGCA
GGTATTGCATATACCGGCATCAATAACGATGTGTA
CACCGAAACCAAACTGTATGTGAGCAAAGACGAAA
AATCGTATATCGATGGCAAAGAAATCGATCTGAAT
AGCGATCGTAGCCCGAGCAAAGTGATCGATAAATT
CAAACATGAAATCCTGATGCGTGTGAATCTGGATG
ATGAAAACAACCTGAGCATTGATAGCCGCAATTTT
AACATTCTGAGCGGTAGCAGCGATAGCGGTGCAGC
AGCACTGGGTGAATGCATTGAAAGCATCTTCGAGT
ACAACATCAACATCTTCACCTTTGAAAATGATCTG
CAGCGTATTAGCGAAAGCGTTGGTCGTAGCCTGTA
TGGTGGTCTGACCGTTAATTATGCAAATGGTCGTG
AAAGCCTGACCGAACCGCTGCTGGAACCGGAAGCA
TTTAACAACTTTACCATCATCGGTGCCCATTTTAA
CATTGATCGCAAACCGAGCAACGAAATCCACGAAA
ACATCATCAAACATGAGAACTATCGCGAACGTATT
AAAAGCGCAGAGCGCAAAGCAAAAAAACTGGAAGA
ACTGAGCCGTAATGCCAACATTAAAGGCATTTTTG
AACTGGCAGAAAGCGATACCGTGGAATATCATAAA
ATGCTGCATGATGTGGGCGTTGATATTATCAATGA
CCGCATGGAAAATCTGATTGAACGCGTGAAAGAGA
TGAAAAACAACTTCTGGAACAGCTATATTGTTACC
GGTGGTCCGAATGTTTTTGTGATCACCGAGAAAAA
AGATGTGGATAAAGCCATGGAAGGTCTGAATGATC
TGTGTGATGATATTCGTCTGCTGAAAGTTGCAGGT
AAACCGCAGGTTATCAGCAAAAACTTCTAATGA.
Also disclosed are variant
or fragments of the M3K gene or
optimized gene sequence of M3K.

Thus, in some aspects, SEQ ID NO:1 represents an optimized gene sequence of M3K.

In some aspects, wild type M3K is a Picrophilus torridus M3K represented by the following nucleic acid sequence

(SEQ ID NO: 3)
ATGGAAAATTACAATGTTAAGACAAGGGCGTTCCC
AACAATAGGCATAATACTGCTTGGTGGGATCTCGG
ATAAAAAGAACAGGATACCGCTGCATACAACGGCA
GGCATAGCATATACTGGTATAAACAATGATGTTTA
CACTGAGACAAAGCTTTATGTATCAAAAGATGAAA
AATGCTATATTGATGGAAAGGAAATTGATTTAAAT
TCAGATAGATCACCATCGAAGGTTATTGATAAATT
CAAGCATGAAATACTTATGAGAGTAAATCTTGATG
ATGAAAATAACCTTTCAATTGATTCAAGGAACTTT
AATATATTAAGTGGCAGCTCAGATTCTGGGGCCGC
TGCACTGGGAGAGTGCATAGAATCAATTTTTGAAT
ACAATATAAATATATTTACATTTGAAAACGATCTT
CAGAGGATATCAGAAAGTGTTGGAAGAAGCCTTTA
CGGTGGTTTAACAGTAAACTATGCCAATGGCAGGG
AATCATTAACAGAGCCATTACTTGAGCCTGAGGCA
TTTAATAACTTTACAATAATTGGTGCACATTTTAA
CATTGATAGAAAACCATCAAATGAGATTCATGAAA
ATATCATAAAACATGAAAATTACAGGGAAAGAATA
AAAAGTGCTGAGAGAAAGGCGAAAAAACTTGAGGA
GCTATCAAGGAATGCAAACATAAAGGGTATCTTTG
AACTTGCAGAATCCGATACAGTGGAATACCATAAA
ATGCTCCATGATGTTGGCGTTGACATAATAAATGA
TAGAATGGAGAACCTCATTGAAAGGGTAAAAGAAA
TGAAAAATAACTTCTGGAATTCATACATAGTTACC
GGCGGCCCGAACGTTTTTGTAATAACAGAGAAAAA
GGACGTTGATAAGGCAATGGAAGGATTAAATGATC
TGTGCGATGATATAAGATTATTAAAAGTTGCAGGA
AAGCCACAGGTCATTTCAAAAAACTTTTAA

In some aspects, wild type M3K is a M3K from one or more of the following species: Acidiplasma cupricumulans, Ferroplasma acidarmanus, Legionella pneumophila, Picrophilus oshimae, Thermoplasma acidophilum, Thermoplasma volcanium, Thermoplasmatales archaeon, Trypansoma brucie, Thermoplasma acidophilum, cuniculiplasma divulgatum, Streptococcus timonensis, Streptococcus parauberis, Streptococcus cristatus, Streptococcus pantholopis, Streptococcus infantis, Mycrobacterium abscessus, peptoniphilu lacrimalis.

Table 1 shows examples of nucleotides from wild type M3K that can be optimized in SEQ ID NO:1. The sequences provided in Table 1 can be variants of the M3K gene that can be used in the disclosed methods.

| Position | Original nucleotide | Optimized nucleotide |
|---|---|---|
| 6 | A | G |
| 9 | T | C |
| 12 | C | T |
| 21 | G | A |
| 24 | A | C |
| 25 | A | C |
| 27 | G | T |
| 30 | G | A |
| 33 | C | T |
| 36 | A | G |
| 39 | A | C |
| 42 | A | T |
| 45 | C | T |
| 48 | A | T |
| 51 | A | T |
| 57 | T | G |
| 63 | G | C |
| 66 | C | T |
| 67 | T | A |
| 68 | C | G |
| 69 | G | C |
| 72 | T | C |
| 78 | G | A |
| 82 | A | C |
| 84 | G | T |
| 87 | A | T |
| 99 | A | C |
| 102 | G | C |
| 108 | C | T |
| 111 | A | T |
| 120 | T | C |
| 123 | T | C |
| 126 | A | C |
| 129 | C | T |
| 132 | T | C |
| 138 | T | G |
| 144 | T | C |
| 147 | G | A |
| 150 | A | C |
| 153 | G | A |
| 156 | T | G |
| 162 | A | G |
| 163 | T | A |
| 164 | C | G |
| 165 | A | C |
| 171 | T | C |
| 179 | G | C |
| 180 | C | G |
| 186 | T | C |
| 192 | A | C |
| 195 | G | A |
| 201 | T | C |
| 205 | T | C |
| 207 | A | G |
| 211 | T | A |
| 212 | C | G |
| 213 | A | C |
| 217 | A | C |
| 219 | A | T |
| 220 | T | A |
| 221 | C | G |
| 222 | A | C |
| 225 | A | G |
| 226 | T | A |
| 227 | C | G |
| 228 | G | C |
| 231 | G | A |
| 234 | T | G |
| 237 | T | C |
| 249 | G | A |
| 258 | A | C |
| 261 | T | G |
| 265 | A | C |
| 267 | A | T |
| 270 | A | G |
| 276 | T | G |
| 288 | T | C |
| 294 | T | G |
| 295 | T | A |
| 296 | C | A |
| 297 | A | C |
| 304 | T | A |
| 305 | C | G |
| 306 | A | C |
| 307 | A | C |
| 309 | G | C |
| 312 | C | T |
| 318 | T | C |
| 321 | A | T |
| 322 | T | C |
| 324 | A | G |
| 327 | T | C |
| 330 | C | T |
| 334 | T | A |
| 335 | C | G |
| 336 | A | C |
| 340 | T | A |
| 341 | C | G |
| 342 | T | C |
| 345 | G | T |
| 348 | C | A |
| 351 | T | A |
| 360 | A | T |
| 363 | G | A |
| 369 | A | T |
| 373 | T | A |
| 374 | C | G |
| 375 | A | C |
| 378 | T | C |
| 381 | T | C |
| 384 | A | G |
| 390 | T | C |
| 393 | A | C |
| 396 | T | C |
| 399 | A | C |
| 402 | T | C |
| 405 | A | C |
| 414 | C | T |
| 420 | T | G |
| 424 | A | C |
| 426 | G | T |
| 429 | A | T |
| 430 | T | A |
| 431 | C | A |
| 432 | A | C |
| 438 | T | C |
| 444 | A | T |
| 445 | A | C |
| 447 | A | T |
| 453 | T | G |
| 456 | C | T |
| 463 | T | C |
| 465 | A | G |
| 468 | A | C |
| 471 | A | T |
| 474 | C | T |
| 480 | C | A |
| 486 | C | T |
| 487 | A | C |
| 489 | G | T |
| 493 | T | A |
| 494 | C | G |
| 495 | A | C |
| 496 | T | C |
| 498 | A | G |
| 501 | A | C |
| 504 | G | A |
| 507 | A | G |
| 508 | T | C |
| 510 | A | G |
| 513 | T | G |
| 516 | G | A |
| 519 | T | G |
| 522 | G | A |
| 531 | T | C |
| 540 | A | C |
| 543 | A | C |
| 546 | T | C |
| 552 | A | C |
| 468 | A | C |
| 470 | A | C |
| 476 | A | G |
| 477 | T | A |
| 478 | C | G |
| 479 | A | C |
| 482 | T | C |
| 485 | G | A |
| 488 | T | C |
| 491 | T | C |
| 497 | T | C |
| 603 | A | C |
| 612 | A | G |
| 615 | T | C |
| 618 | C | T |
| 619 | A | C |
| 621 | G | C |
| 625 | A | C |
| 627 | A | T |
| 630 | A | T |
| 636 | T | C |
| 639 | T | A |
| 643 | A | C |
| 645 | A | C |
| 648 | G | A |
| 651 | G | A |
| 660 | T | G |
| 663 | G | A |
| 666 | G | A |
| 669 | A | G |
| 670 | T | A |
| 671 | C | G |
| 672 | A | C |
| 673 | A | C |
| 675 | G | T |
| 681 | A | C |
| 687 | A | T |
| 690 | G | A |
| 693 | T | C |
| 696 | C | T |
| 705 | T | G |
| 712 | T | A |
| 713 | C | G |
| 720 | A | C |
| 729 | C | T |
| 741 | C | G |
| 750 | T | G |
| 759 | C | T |
| 762 | A | T |
| 765 | A | C |
| 771 | T | C |
| 772 | A | C |
| 774 | A | C |
| 780 | G | A |
| 783 | C | T |
| 786 | C | G |
| 793 | A | C |
| 795 | G | C |
| 798 | A | G |
| 804 | T | G |
| 813 | T | C |
| 825 | T | C |
| 826 | T | A |
| 827 | C | G |
| 828 | A | C |
| 831 | C | T |
| 834 | A | T |
| 843 | C | T |
| 846 | C | T |
| 852 | C | T |
| 861 | A | G |
| 864 | A | C |
| 867 | A | C |
| 876 | G | A |
| 879 | C | T |
| 882 | T | G |
| 888 | G | A |
| 891 | A | C |
| 900 | A | T |
| 901 | T | C |
| 903 | A | G |
| 915 | C | T |
| 924 | A | T |
| 925 | A | C |
| 927 | A | T |
| 928 | T | C |
| 930 | A | G |
| 931 | T | C |
| 933 | A | G |
| 945 | A | T |
| 948 | G | A |
| 951 | A | G |
| 957 | C | T |
| 960 | T | C |
| 961 | T | A |
| 962 | C | G |
| 963 | A | C |
| 972 | T | C |
| 976 | — | T |
| 977 | — | G |
| 978 | — | A |

In some aspects, disclosed are M3K gene sequences comprising at least 85, 90, 95, or 99% identity to the sequence of SEQ ID NO:1. In some aspects, disclosed are M3K gene sequences comprising at least 90% identity to the sequence of SEQ ID NO:1. In some aspects, the M3K gene sequence is 100% identical to SEQ ID NO:1 at the optimized nucleotides shown in Table 1. Thus, in some aspects, the differences between SEQ ID NO:1 and a disclosed M3K gene can be present at any nucleotide besides those listed in Table 1.

4. MVD gene

MVD, a key enzyme in the MVA pathway, is an ATP-dependent enzyme that catalyzes the conversion of, by decarboxylating, 3-hydroxyisovalerate (3-HIV) to isobutene.

In some aspects, an E. coli optimized MVD can comprise the following nucleic acid sequence

(SEQ ID NO: 2)
ATGACCGTTTATACCGCAAGCGTTACCGCACCGGT
TAATATTGCAACCCTGAAATATTGGGGTAAACGTG
ATACCAAACTGAATCTGCCGACCAATAGCAGCATT
AGCGTTACCCTGAGCCAGGATGATCTGCGTACCCT
GACCAGCGCAGCAACCGCACCGGAATTTGAACGTG
ATACCCTGTGGCTGAATGGTGAACCGCATAGCATT
GATAATGAACGTACCCAGAATTGTCTGCGTGATCT
GCGTCAGCTGCGTAAAGAAATGGAAAGCAAAGATG
CAAGCCTGCCGACCCTGAGCCAGTGGAAACTGCAT
ATTGTTAGCGAAAATAATTTTCCGACCGCAGCAGG
TCTGGCAAGCAGCGCAGCAGGTTTTGCAGCACTGG
TTAGCGCAATTGCAAAACTGTATCAGCTGCCGCAG
AGCACCAGCGAAATTAGCCGTATTGCACGTAAAGG
TAGCGGTAGCGCATGTCGTAGCCTGTTTGGTGGTT
ATGTTGCATGGGAAATGGGTAAAGCAGAAGATGGT
CATGATAGCATGGCAGTTCAGATTGCAGATAGCAG
CGATTGGCCGCAGATGAAAGCATGTGTTCTGGTTG
TTAGCGATATTAAAAAAGATGTTAGCAGCACCCAG
GGTATGCAGCTGACCGTTGCAACCAGCGAACTGTT
TAAAGAACGTATTGAACATGTTGTTCCGAAACGTT
TTGAAGTTATGCGTAAAGCAATTGTTGAAAAAGAT
TTCGCAACCTTTGCAAAAGAAACCATGATGGATAG
CAATAGCTTTCATGCAACCTGTCTGGATAGCTTTC
CGCCGATTTTTTATATGAATGATACCAGCAAACGC
ATTATTAGCTGGTGTCATACCATTAATCAGTTTTA
TGGTGAAACCATTGTGGCATATACCTTTGATGCAG
GTCCGAATGCAGTTCTGTATTATCTGGCAGAAAAT
GAAAGCAAACTGTTTGCATTTATCTACAAACTGTT
CGGTAGCGTTCCGGGTTGGGATAAAAAATTTACCA
CCGAACAGCTGGAAGCATTTAATCATCAGTTTGAA
AGCAGCAATTTCACCGCACGTGAACTGGATCTGGA
ACTGCAGAAAGATGTTGCACGTGTTATTCTGACCC
AGGTTGGTAGCGGTCCGCAGGAAACCAATGAAAGC
CTGATTGATGCAAAAACCGGTCTGCCGAAAGAATA
A.
Also disclosed are variant or fragments
of the MVD gene or optimized gene
sequence of MVD.

In some aspects, wild type MVD is a Saccharomyces cerevisiae MVD represented by the following nucleic acid sequence

(SEQ ID NO: 4)
ATGACCGTTTACACAGCATCCGTTACCGCACCCGT
CAACATCGCAACCCTTAAGTATTGGGGGAAAAGGG
ACACGAAGTTGAATCTGCCCACCAATTCGTCCATA
TCAGTGACTTTATCGCAAGATGACCTCAGAACGTT
GACCTCTGCGGCTACTGCACCTGAGTTTGAACGCG
ACACTTTGTGGTTAAATGGAGAACCACACAGCATC
GACAATGAAAGAACTCAAAATTGTCTGCGCGACCT
ACGCCAATTAAGAAAGGAAATGGAATCGAAGGACG
CCTCATTGCCCACATTATCTCAATGGAAACTCCAC
ATTGTCTCCGAAAATAACTTTCCTACAGCAGCTGG
TTTAGCTTCCTCCGCTGCTGGCTTTGCTGCATTGG
TCTCTGCAATTGCTAAGTTATACCAATTACCACAG
TCAACTTCAGAAATATCTAGAATAGCAAGAAAGGG
GTCTGGTTCAGCTTGTAGATCGTTGTTTGGCGGAT
ACGTGGCCTGGGAAATGGGAAAAGCTGAAGATGGT
CATGATTCCATGGCAGTACAAATCGCAGACAGCTC
TGACTGGCCTCAGATGAAAGCTTGTGTCCTAGTTG
TCAGCGATATTAAAAAGGATGTGAGTTCCACTCAG
GGTATGCAATTGACCGTGGCAACCTCCGAACTATT
TAAAGAAAGAATTGAACATGTCGTACCAAAGAGAT
TTGAAGTCATGCGTAAAGCCATTGTTGAAAAAGAT
TTCGCCACCTTTGCAAAGGAAACAATGATGGATTC
CAACTCTTTCCATGCCACATGTTTGGACTCTTTCC
CTCCAATATTCTACATGAATGACACTTCCAAGCGT
ATCATCAGTTGGTGCCACACCATTAATCAGTTTTA
CGGAGAAACAATCGTTGCATACACGTTTGATGCAG
GTCCAAATGCTGTGTTGTACTACTTAGCTGAAAAT
GAGTCGAAACTCTTTGCATTTATCTATAAATTGTT
TGGCTCTGTTCCTGGATGGGACAAGAAATTTACTA
CTGAGCAGCTTGAGGCTTTCAACCATCAATTTGAA
TCATCTAACTTTACTGCACGTGAATTGGATCTTGA
GTTGCAAAAGGATGTTGCCAGAGTGATTTTAACTC
AAGTCGGTTCAGGCCCACAAGAAACAAACGAATCT
TTGATTGACGCAAAGACTGGTCTACCAAAGGAATA
A.

In some aspects, any known MVD sequence can be used.

Table 2 shows the nucleotides from wild type MVD (SEQ ID NO:4) that can be optimized in SEQ ID NO:2. The sequences provided in Table 2 can be variants of the MVD gene that can be used in the disclosed methods.

TABLE 2

| Position | Original nucleotide | Optimized nucleotide |
|---|---|---|
| 12 | C | T |
| 15 | A | C |
| 19 | T | A |
| 20 | C | G |
| 33 | C | G |
| 36 | C | T |
| 39 | C | T |
| 42 | C | T |
| 51 | T | G |
| 54 | G | A |
| 63 | G | T |
| 67 | A | C |
| 69 | G | T |
| 72 | C | T |
| 75 | G | C |
| 78 | G | A |
| 79 | T | C |
| 90 | C | G |
| 97 | T | A |
| 98 | C | G |
| 99 | G | C |
| 100 | T | A |
| 101 | C | G |
| 105 | A | T |
| 106 | T | A |
| 107 | C | G |
| 108 | A | C |
| 111 | G | T |
| 114 | T | C |
| 115 | T | C |
| 117 | A | G |
| 118 | T | A |
| 119 | C | G |
| 120 | G | C |
| 123 | A | G |
| 129 | C | T |
| 132 | C | G |
| 133 | A | C |
| 135 | A | T |
| 138 | G | C |
| 139 | T | C |
| 145 | T | A |
| 146 | C | G |
| 147 | T | C |
| 150 | G | A |
| 153 | T | A |
| 156 | T | C |
| 162 | T | G |
| 165 | G | A |
| 174 | C | T |
| 177 | C | T |
| 180 | T | C |
| 181 | T | C |
| 187 | T | C |
| 189 | A | G |
| 195 | A | T |
| 201 | A | G |
| 204 | C | T |
| 210 | C | T |
| 213 | C | T |
| 220 | A | C |
| 222 | A | T |
| 225 | T | C |
| 228 | A | G |
| 240 | C | T |
| 243 | C | T |
| 246 | A | G |
| 249 | C | T |
| 252 | A | G |
| 253 | T | C |
| 255 | A | G |
| 256 | A | C |
| 258 | A | T |
| 261 | G | A |
| 271 | T | A |
| 272 | C | G |
| 273 | G | C |
| 276 | G | A |
| 279 | C | T |
| 282 | C | A |
| 283 | T | A |
| 284 | C | G |
| 285 | A | C |
| 286 | T | C |
| 291 | C | G |
| 294 | A | C |
| 295 | T | C |
| 297 | A | G |
| 298 | T | A |
| 299 | C | G |
| 300 | T | C |
| 303 | A | G |
| 312 | C | G |
| 315 | C | T |
| 321 | C | T |
| 322 | T | A |
| 323 | C | G |
| 333 | C | T |
| 339 | T | G |
| 342 | A | C |
| 348 | T | A |
| 352 | T | C |
| 354 | A | G |
| 357 | T | A |
| 358 | T | A |
| 359 | C | G |
| 361 | T | A |
| 362 | C | G |
| 366 | T | A |
| 369 | T | A |
| 372 | C | T |
| 378 | T | A |
| 382 | T | C |
| 387 | C | T |
| 388 | T | A |
| 389 | C | G |
| 390 | T | C |
| 399 | T | A |
| 402 | G | A |
| 403 | T | C |
| 405 | A | G |
| 408 | C | T |
| 411 | A | G |
| 412 | T | C |
| 414 | A | G |
| 417 | A | G |
| 421 | T | A |
| 422 | C | G |
| 423 | A | C |
| 426 | T | C |
| 427 | T | A |
| 428 | C | G |
| 429 | A | C |
| 435 | A | T |
| 436 | T | A |
| 437 | C | G |
| 438 | T | C |
| 439 | A | C |
| 441 | A | T |
| 444 | A | T |
| 448 | A | C |
| 450 | A | T |
| 453 | G | A |
| 456 | G | T |
| 457 | T | A |
| 458 | C | G |
| 459 | T | C |
| 463 | T | A |
| 464 | C | G |
| 465 | A | C |
| 468 | T | A |
| 472 | A | C |
| 474 | A | T |
| 475 | T | A |
| 476 | C | G |
| 477 | G | C |
| 478 | T | C |
| 486 | C | T |
| 489 | A | T |
| 492 | C | T |
| 495 | G | T |
| 498 | C | A |
| 510 | A | T |
| 555 | T | A |
| 532 | T | A |
| 533 | C | G |
| 543 | A | T |
| 546 | A | G |
| 549 | C | T |
| 555 | C | T |
| 559 | T | A |
| 560 | C | G |
| 561 | T | C |
| 564 | C | T |
| 570 | T | G |
| 582 | T | A |
| 588 | C | T |
| 591 | A | G |
| 597 | C | T |
| 612 | G | A |
| 518 | G | T |
| 621 | T | C |
| 622 | T | A |
| 623 | C | G |
| 627 | T | C |
| 639 | A | G |
| 640 | T | C |
| 648 | G | T |
| 655 | T | A |
| 656 | C | G |
| 663 | A | G |
| 673 | A | C |
| 675 | A | T |
| 687 | C | T |
| 690 | A | T |
| 693 | A | G |
| 696 | G | A |
| 697 | A | C |
| 699 | A | T |
| 708 | C | T |
| 720 | C | A |
| 741 | C | A |
| 753 | G | A |
| 759 | A | C |
| 769 | T | A |
| 770 | C | G |
| 774 | C | T |
| 775 | T | A |
| 776 | C | G |
| 777 | T | C |
| 780 | C | T |
| 786 | C | A |
| 789 | A | C |
| 793 | T | C |
| 798 | C | T |
| 799 | T | A |
| 800 | C | G |
| 801 | T | C |
| 804 | C | T |
| 807 | T | G |
| 810 | A | G |
| 813 | A | T |
| 816 | C | T |
| 819 | C | T |
| 828 | C | T |
| 831 | T | C |
| 832 | T | A |
| 833 | C | G |
| 837 | G | A |
| 840 | T | C |
| 843 | C | T |
| 846 | C | T |
| 849 | T | C |
| 855 | C | T |
| 858 | C | T |
| 876 | C | T |
| 879 | A | T |
| 885 | A | C |
| 888 | C | T |
| 891 | T | G |
| 897 | C | T |
| 900 | G | C |
| 915 | A | G |
| 921 | T | A |
| 924 | G | T |
| 925 | T | C |
| 930 | C | T |
| 933 | C | T |
| 934 | T | C |
| 936 | A | G |
| 939 | T | A |
| 948 | G | A |
| 949 | T | A |
| 950 | C | G |
| 951 | G | C |
| 957 | C | G |
| 972 | T | C |
| 976 | T | C |
| 981 | T | C |
| 984 | C | T |
| 985 | T | A |
| 986 | C | G |
| 987 | T | C |
| 993 | T | G |
| 996 | A | T |
| 1002 | C | T |
| 1005 | G | A |
| 1014 | T | C |
| 1017 | T | C |
| 1020 | G | A |
| 1026 | T | G |
| 1029 | G | A |
| 1032 | T | A |
| 1035 | C | T |
| 1038 | C | T |
| 1044 | A | G |
| 1051 | T | A |
| 1052 | C | G |
| 1053 | A | C |
| 1054 | T | A |
| 1055 | C | G |
| 1056 | T | C |
| 1059 | C | T |
| 1062 | T | C |
| 1065 | T | C |
| 1075 | T | C |
| 1083 | T | G |
| 1086 | G | A |
| 1087 | T | C |
| 1092 | A | G |
| 1095 | G | A |
| 1104 | C | A |
| 1105 | A | C |
| 1107 | A | T |
| 1110 | G | T |
| 1114 | T | C |
| 1116 | A | G |
| 1119 | T | C |
| 1122 | A | G |
| 1125 | C | T |
| 1129 | T | A |
| 1130 | C | G |
| 1131 | A | C |
| 1134 | C | T |
| 1137 | A | G |
| 1140 | A | G |
| 1146 | A | C |
| 1149 | C | T |
| 1153 | T | A |
| 1154 | C | G |
| 1155 | T | C |
| 1156 | T | C |
| 1164 | C | T |
| 1170 | G | A |
| 1173 | T | C |
| 1179 | A | G |
| 1182 | A | G |
| 1185 | G | A |

In some aspects, disclosed are MVD gene sequences comprising at least 85, 90, 95, or 99% identity to the sequence of SEQ ID NO:2. In some aspects, disclosed are MVD gene sequences comprising at least 90% identity to the sequence of SEQ ID NO:2. In some aspects, the MVD gene sequence is 100% identical to SEQ ID NO:2 at the optimized nucleotides shown in Table 2. Thus, in some aspects, the differences between SEQ ID NO:2 and a disclosed MVD gene can be present at any nucleotide besides those listed in Table 2.

### C. Vectors

Disclosed are vectors comprising any of the nucleic acid sequences and constructs disclosed herein.

In some aspects, the vector can be a viral vector. In some aspects, the vector can be a plasmid. In some aspects, the vector can be an expression vector.

The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

In some aspects, the vector can be a viral vector. For example, the viral vector can be a retroviral vector. In some aspects, the vector can be a non-viral vector, such as a DNA based vector.

### 1. Viral and Non-Viral Vectors

There are a number of compositions and methods which can be used to deliver the disclosed nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Expression vectors can be any nucleotide construction used to deliver genes or gene fragments into cells (e.g., a plasmid), or as part of a general strategy to deliver genes or gene fragments, e.g., as part of recombinant retrovirus or adenovirus (Ram et al. Cancer Res. 53:83-88, (1993)). For example, disclosed herein are expression vectors comprising a nucleic acid sequence capable of encoding a VMD2 promoter operably linked to a nucleic acid sequence encoding Rap1a.

The “control elements” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif) or pSPORT1 plasmid (Gibco BRL, Gaithersburg, Md.) and the like may be used. If it is necessary to generate a cell line that contains multiple copies of the sequence encoding a polypeptide, vectors based on SV40 or EBV may be advantageously used with an appropriate selectable marker.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Optionally, the promoter or enhancer region can act as a constitutive promoter or enhancer to maximize expression of the polynucleotides of the invention. In certain constructs the promoter or enhancer region be active in all eukaryotic cell types, even if it is only expressed in a particular type of cell at a particular time.

The expression vectors can include a nucleic acid sequence encoding a marker product. This marker product can be used to determine if the gene has been delivered to the cell and once delivered is being expressed. Marker genes can include, but are not limited to the E. coli lacZ gene, which encodes β-galactosidase, and the gene encoding the green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

Another type of selection that can be used with the composition and methods disclosed herein is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

As used herein, plasmid or viral vectors are agents that transport the disclosed nucleic acids, such as a nucleic acid sequence capable of encoding one or more of the disclosed peptides into the cell without degradation and include a promoter yielding expression of the gene in the cells into which it is delivered. In some embodiments the nucleic acid sequences disclosed herein are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction abilities (i.e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I. M., Retroviral vectors for gene transfer. In Microbiology, Amer. Soc. for Microbiology, pp. 229-232, Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol., 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides of the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors. In addition, the disclosed nucleic acid sequences can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subjects lung cells. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

### D. Recombinant Cells

Disclosed are recombinant cells comprising the nucleic acids, nucleic acid constructs and peptides and proteins disclosed herein. In an aspect, disclosed herein are recombinant cells comprising a nucleic acid sequence, wherein the nucleic acid sequence comprises a first E. coli homology region, wherein the first E. coli homology region comprises a PAM mutation; a constitutive promoter; a M3K gene; a MVD gene; and a second E. coli homology region.

Disclosed are recombinant cells comprising a nucleic acid sequence, wherein the nucleic acid sequence comprises a first E. coli homology region, wherein the first E. coli homology region comprises a PAM mutation; a constitutive promoter; a M3K gene comprising the sequence of SEQ ID NO:1; a MVD gene comprising the sequence of SEQ ID NO:2; and a second E. coli homology region.

Disclosed are recombinant cells comprising a nucleic acid sequence, wherein the nucleic acid sequence comprises a first E. coli homology region, wherein the first E. coli homology region comprises a PAM mutation; a constitutive promoter; a M3K gene comprising a sequence having at least 90% identity to the sequence of SEQ ID NO:1; a MVD gene comprising a sequence having at least 90% identity to the sequence of SEQ ID NO:2; and a second E. coli homology region.

In some aspects, the disclosed nucleic acid sequences are integrated into the genome of the recombinant cells. In some aspects, non-integrated plasmid nucleic acid sequences can often require an antibiotic resistance gene for selection. Therefore, integration of the disclosed nucleic acid sequences into the genome of the recombinant cells can remove the need for using antibiotics.

In some aspects, the recombinant cells can be bacterial cells. In some aspects, the bacteria is E. coli.

In some aspects, the M3K gene and MVD gene are in a region of the recombinant cell's genome known to have higher expression levels. For example, in some aspects, the region of the genome known to have higher expression levels is the safe site 9 region of E. coli.

In some aspects, the M3K gene and the MVD gene are operably linked.

In some aspects, the M3K gene and MVD gene are controlled by the constitutive promoter. The constitutive promoter can be, for example, a 16S rRNA promoter or a T7A1 promoter. Any of the constitutive promoters disclosed herein can be used to control the M3K and MVD genes.

### E. Methods of Making Recombinant Cells

Disclosed are methods of making recombinant cells comprising administering any one of the disclosed linear nucleic acid sequences to a cell, wherein the cell incorporates the linear nucleic acid sequence into its cellular genome.

In some aspects, the incorporation of the disclosed nucleic acid sequences into the cellular genome occurs through homologous recombination using the first and second E. coli homology regions of the nucleic acid sequence.

In some aspects, the disclosed methods of making recombinant cells are methods of making recombinant bacterial cells.

In some aspects, the disclosed methods can further comprise administering a safe site 9 (SS9) specific gRNA to the recombinant cell. In some aspects, the gRNA targets the PAM site located within SS9. Thus, if the PAM site changes during the recombination then the gRNA cannot direct the Cas9 enzyme there to cut the DNA. In some aspects, the PAM site changes during recombination because the first and/or second homology regions have a mutated PAM site so when then first and/or second homology regions recombine into the cellular genome the wild type PAM site is no longer present, only the mutated PAM site is present and the gRNA cannot direct the Cas9 enzyme to the DNA.

In some aspects, the use of gRNA and Cas9 enzyme cutting the DNA can be used as a selection process for those DNA sequences that underwent homologous recombination. For example, if homologous recombination occurred and the nucleic acid sequence comprising the first and second homology regions recombined into the cells genome, the cellular genome would not be cut by the addition of the specific gRNAs and cas enzymes disclosed herein. The first and second homology regions can comprise mutant PAM sites and therefore the homologous recombination of the first and second homology regions into the cells genome, eliminates the wild type PAM sites from the cell's genome and replaces it with the mutated PAM sites from the first and second homology regions. Thus, the cell's genome is not cut by the addition of gRNA and cas enzyme. This process of adding the specific gRNAs and cas enzyme can be used to select for only those cells that underwent homologous recombination because those cells that did not undergo homologous recombination will die once the cas enzyme cuts the genome at the wild type PAM site.

In some aspects, the recombinant cells comprise Cas9 or a gene encoding Cas9. Thus, Cas9 can be expressed within the cell without having to exogenously add it. As used herein, “Cas9” can be wild type Cas9 proteins (i.e., those that occur in nature), modified Cas9 proteins (i.e., Cas9 protein variants), or fragments of wild type or modified Cas9 proteins. Cas9 proteins can also be active variants or fragments with respect to catalytic activity of wild type or modified Cas9 proteins. In some aspects, the recombinant cells comprise any Cas protein, wild type or modified.

In some aspects, only the cells that incorporate the linear nucleic acid sequence into the cellular genome will remain viable.

### F. Methods of Producing Isobutene

Disclosed are methods of producing isobutene comprising growing any one of the disclosed recombinant bacteria cells comprising a nucleic acid sequence comprising a constitutive promoter; a M3K gene and a MVD gene under conditions suitable for bacterial growth and expression of M3K and MVD, wherein the MVD decarboxylates 3-hydroxyisovalerate (3-HIV) to isobutene, and wherein the M3K catalyzes the phosphorylation of 3-HIV into an unstable 3-phosphate intermediate that undergoes spontaneous decarboxylation to isobutene.

In some aspects, the cells can be grown in wastewater from a water treatment plant for production of the isobutene. Thus, in some aspects, traditional broth or media can be substituted for wastewater.

### G. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising a first E. coli homology region, a constitutive promoter; a M3K gene; a MVD gene; and a second E. coli homology region. The kits also can contain a vector.

### EXAMPLES

### A. Introduction

In the modern world, the impacts of fossil fuels are inescapable. In addition to generating energy, fossil fuel products are used to create high-value industrial chemicals and polymers that permeate society. These polymers are used to create plastic products around the world and production is increasing. The world produced an estimated 322 million tons of plastic in 2015. Currently, the petrochemicals utilized around the world are produced predominantly via steam cracking of crude petroleum products. This process requires high temperatures, pressures, and anoxic conditions. The steam cracking process is very energetically demanding, requiring 7-15% of the oil input simply for heat generation. This accounts for 14% of the total energy consumption of the U.S. Additionally, molten salt is used to reduce coke formation in the refinement process leading to additional requirements for contaminant disposal. Producing petrochemicals in this fashion emits massive quantities of greenhouse gasses (GHG) and potential environmental contaminants that have a myriad of effects on the environment, economies, and species as a whole. Research is needed on ways to sustainably produce these petrochemicals while reducing the negative effects of current production processes.

Isobutene (isobutylene, 3-methylpropene) is one such petrochemical that can benefit from a sustainable production process. Isobutene is a widely used petrochemical with a global market value of—22 Billion USD/year and is expected to rise to—31 billion USD/year by 2024. It is used to create fuel additives such as isooctane, methyl tert-butyl ether (MTBE), and ethyl tert-butyl ether (ETBE) through electrophilic additions of isobutane, methanol, and ethanol, respectively. More importantly isobutene can be polymerized to make isobutyl rubber used in tires, gaskets, gum, hoses, and more (Table 3). Isobutyl rubber is of special significance because it is the only manufactured gas tight synthetic rubber. Isobutene can also be polymerized with isoprene to make isobutyl-isoprene rubber, which is also widely used, but can vary in permeability with the ratio of isobutene to isoprene. Due to the economic importance of isobutene, maintaining or increasing production to meet demands is necessary.

TABLE 3
Industrial Products Synthesized from Isobutene

| Product | Examples |
|---|---|
| Insecticides | Aldicarb |
| Fuel Additives | MTBE |
| | ETBE |
| | isooctane |
| Polymers | Butyl rubber |
| | Isobutylene-isoprene rubber (IIR) |
| | Methyl methacylate |
| Antioxidants | Butylated hydroxyanisole |
| | Butylated hydroxytoluene |
| Various Uses | Tert-butylamine |
| | Tert-butanol |
| | Tert butyl acetate |
| | Diisobutyl esters |
| | Isobutylene-isoprene co-polymer |

### 1. Bioproduction of Industrial Chemicals

One avenue of research that can be more carbon neutral and less polluting is the biological production of industrially important chemicals. While switching production of petrochemicals from steam-cracking crude oil to a renewable production method would not alleviate all emissions, it would redress one of the major sources. There is a wide range of petrochemicals that are produced in varying quantities by microbes, which makes biological production an alluring avenue of research; however, harnessing bioproduction of these compounds has proved challenging. Production of alcohol by microbes has been taken advantage by humans for well over a thousand years, but production of other high energy compounds such as ethers, esters, alkanes, and alkenes are just now garnering increased interest. Many industrially important compounds, such as ethanol, butanol, methane, 1,2-propanediol, and 3-hydroxypropionate can be generated by microbial fermentation and for some of these compounds, large scale biological production has been implemented with varying degrees of success. For many other compounds, biological production has been demonstrated but has yet to move beyond the benchtop. Longer chain hydrocarbons commonly found in microbes are made through either elongation-decarboxylation reactions or head to head condensation reactions with fatty acids. Enhanced production of fatty acids for conversion to longer chain hydrocarbons is being investigated as a method to produce petrol type fuels from microbes.

Microbial production of ethylene has been detected in a wide variety of bacteria and fungi. It is especially prevalent in plant-associated microbes, possibly due to ethylenes' role as a plant growth regulatory hormone. Microbial production of ethylene is carried out through the KMBA pathway, which converts methionine to 2-oxo acid 2-oxo-4-methylthiobutyric acid (KMBA), then to ethylene. Bioproduction of isoprene has been seen in various bacterial groups, from actinomycetes to Pseudomonas and Bacillus. Microbial production of isobutene has been detected in all microbial domains of life, Archaea, Bacteria and Fungi. Microbial production of isobutene proceeds from the breakdown products of leucine and valine catabolism.

Current research in bioproduction of industrial chemicals has focused on optimization of production organisms. This optimization happens through genetic engineering and careful manipulations of growth conditions to overcome inefficiencies. Next Generation Sequencing (NGS) coupled with advancements in genome engineering has allowed for discovery of novel enzymes for biofuel production and incorporation into cell factories to produce more petrochemicals through biological means. There is now validated biological production at varying levels of methane, propane, isobutanol, ethylene, isobutene, isoprene and more. By taking advantage of newly elucidated CRISPR/Cas systems, it is possible to carry out genomic insertions in bacteria without relying on antibiotic resistance as a counter selection. With these new technologies, it has become possible to insert whole pathways for biofuel production in host strains in single recombination events without scarring the host genome.

There has been significant work over the last two decades to bring biofuels to market in higher quantities at more affordable prices. Much of the effort has been in the production of biofuels or biodiesels from crops such as soybeans, corn, or palm trees. Using yeast to produce ethanol through fermenting corn is a prime example of a process that has become economically viable. However, designating this process economically viable must carry a significant caveat due to the high level of government subsidization for production of ethanol from corn. Furthermore, using food crops as a feedstock for biofuels is increasingly problematic as requirements for food production are expected to rise by up to 60% over the next 40 years. This is now leading nations away from subsidizing the farming of food crops for biofuel production. For example, the European Union in 2018 voted to cease subsidies for palm oil production for biodiesels. This move away from food crops means cheaper replacement feedstocks are needed for biofuel production. More recently developed biofuel processes either use more recalcitrant feedstocks (lignocellulosic biomass) or use engineered autotrophs for overproduction of fatty acids from atmospheric CO₂. These later generation biofuel techniques seek to first improve economic viability by lowering the initial cost threshold through low cost or free carbon sources.

Wastewater is one potential feedstock for the production of bioproducts that can help to reduce the cost and increase economic viability. Due to the relatively high concentrations of lipids present in sewage sludge, (2-12% wt for secondary sludge and 15-30% wt for primary sludge) use of wastewater as a feedstock would provide high energy compounds i.e., fatty acids for metabolism. The viability of wastewater, either from farms, processing plants or municipalities for use as a feedstock has been demonstrated in production of several biofuels, such as hydrogen, methane, butanol, and acetone. Growing algae in wastewater from farms and municipalities was shown to be effective at both removing dissolved nitrogen and phosphorous from the effluent as well as for converting solid mass into lipid biomass. The high lipid content of the algal biomass means that it can be condensed into biodiesel with the application of heat and pressure. Another potential mechanism to convert biomass to biofuels, is anaerobic digestion of the microalgal biomass grown on wastewater effluent for methane production. Wastewaters from farms and agricultural processing have been used as feedstocks for biological production of hydrogen and methane. The wide range of biofuels that can be produced from microbial growth on wastewaters demonstrates its viability as a feedstock.

Even with cheaper feedstocks it is a struggle for bioproduction of isobutene and other petrochemicals to reach economic viability. This is primarily due to inefficient pathways that produce low titers. Optimization of all stages of the process must occur, which will require multidisciplinary teams. For example, teams of engineers, modelers, and synthetic biologists can work together to design a complete optimized system from feedstock to the bioreactor, to streamlining genetic engineering through deriving mathematical models of metabolic networks to predict energetic favorability. To reduce or alleviate the need for use of expensive sugar feedstocks, the use of lignocellulosic materials such as corn stover for biofuels feedstocks is particularly appealing. There have been some promising studies engineering E. coli to degrade other recalcitrant structural polysaccharides such as pectin and cellobiose. Researchers have shown that disruption of methylglyoxal synthase improves co-metabolism of pentoses and hexoses in E. coli. This deactivation helps alleviate the metabolic throttling of the carbon catabolite repression (CCR) systems in E. coli. The use of biofilm-based bioreactors rather than liquid suspension cultures for microalgal production of biofuels has also been investigated. Through cultivation of biofilms rather than planktonic cells, researchers produced equivalent quantities of biofuel precursors as liquid suspension but with a significant reduction in the volume of media used for culturing. Multidisciplinary teams that combine these methods can increase metabolic efficiency, improve degradation of recalcitrant feedstocks and reduce culture volumes. These, in turn, will decrease the cost of biofuel production and improve economic viability.

### 2. Tolerance Engineering for Enhanced Bioproduction

Genetic manipulation to increase solvent tolerance has been demonstrated as an effective method for increasing production titers for biofuels. Most biofuel-compounds cause damage to cellular membranes and nucleic acids. Accordingly, as the concentration of the biofuel increases it can inhibit the growth of the producer. Increasing tolerance to these compounds can allow for production of higher concentrations of before inhibition happens. Tolerance to a compound can be achieved in more than one way. Repeated and prolonged exposure to biofuels can be used to drive selection for mutants with increased tolerance to the target compound. Additionally, identifying and characterizing genes that show increased transcription following exposure to biofuels can help elucidate targets for mutation or overexpression. A method that has proven effective for both increased tolerance and, in some cases production, of industrial chemicals is overexpression of efflux pumps. Efflux pumps are a well-reasoned target to overexpress or optimize as they will remove the biofuel from the intracellular milieu preventing further intracellular damage. Isopentenol tolerance and production both were increased through overexpression of several genes that had been upregulated in response to isopentenol exposure. These genes ranged from chaperones ibpA to transporters mdlB to transcriptional activators metR. Clostridium acetobutylicum was engineered for enhanced production and tolerance to butanol and acetone through overexpression of chaperones groELS.

### B. Bioproduction of Isobutene

### 1. Introduction

Several biological pathways for production of isobutene have been discovered that utilize a variety of precursors (FIG. 1). Decarboxylation of isovalerate by cytochrome P450 has been shown to produce isobutene at a rate of 11 nmol*min-1 *mg protein-1. This reaction required NADPH, 02, and a second cytochrome P450 enzyme functioning as an NADPH reductase. Production of isobutene through dehydration of isobutanol via an oleate hydratase is known. Unfortunately, no information on rates of production is known, thus limiting the comparison to production via other enzymatic methods.

Two enzymes that have previously been shown to catalyze the production of isobutene from 3-hydroxyisovalerate (3-HIV) are mevalonate diphosphate decarboxylase (MVD) and mevalonate-3-kinase (M3K), and this study is focused on isobutene production through expression of these two enzymes. Isobutene production from purified MVD on 3-HIV has been reported as high as 6.44±0.95_pmol*min-1 *mg protein-1. Isobutene production from purified M3K has been reported reaching 26±2 pmol*min-1 *mg protein at 30° C. and 2,880±140 pmol*min-1 *mg protein at 50° C. The highest biological rates of isobutene production have been through action of M3K on 3-HIV, with production from MVD being the second highest. Both enzymes have been shown to each catalyze production of isobutene from reactions with 3-HIV through separate mechanisms. MVD reacts with 3-HIV in an ATP dependent reaction and after 3-HIV is phosphorylated, catalyzes the removal of a carbonyl group with concomitant release of inorganic phosphate to yield isobutene (Reaction 1). Similarly, M3K acts to catalyze the addition of a phosphate group to 3-HIV to produce 3-phosphoisovalerate (3-PIV) which undergoes spontaneous decarboxylation with concomitant release of inorganic phosphate to produce isobutene (Reaction 2).

C₅H₉O₃+ATP→C₄H₈+CO₂+Pi (1)

C₅H₉O₃+ATP→C₅H₈O₃P→C₄H₈+CO₂+Pi (2)

Due to the differing modes of action of these two enzymes, expression of both enzymes in one organism can enhance production above that achieved by an organism only expressing one of the two enzymes. Significant improvements in biological production of isobutene are still needed as current rates for production are approximately 10⁶ times lower than needed to be economically viable.

### 2. Isobutene Production

Described herein is the integration of a nucleic acid sequence comprising M3K and MVD into the E. coli genome. FIG. 2 shows an example of a nucleic acid sequence comprising both M3K and MVD being inserted into an E. coli genome at a safe site 9 (SS9) region.

Biological production of isobutene from both MVD and M3K has been previously demonstrated in whole cell culture and cell free extracts. The efficiency of isobutene production in E. coli expressing MVD and M3K from the central chromosome was unknown before this study. E. coli MG1655 was engineered through homologous recombination, with Cas9 mediated counterselection to express MVD and M3K from SS9 in its' chromosome. After all introduced plasmids were cured, successful genomic engineering was validated by the detection of isobutene in the headspace. No isobutene was detected in the headspace of controls from non-recombinant cultures. Isobutene production has previously been demonstrated to be linear over the first 48 hours of growth in E. coli cultures expressing MVD or M3K from plasmids and this assumption was used for calculating production rates. Isobutene production by the engineered strain was measured after 24 hours. The highest production measured was 144.68 pmol*min⁻¹ *g cells⁻¹ and the average production rate from the engineered strain was 135.70±7.78pmol*min⁻¹*g cells⁻¹ (Table 4). This production value remains significantly lower than production from plasmid-based expression.

TABLE 4
Biological Isobutene Production from Culture

| | Isobutene Production | |
|---|---|---|
| Production Method | pmol/min*g cells | Source |
| Whole Cell from Genome | 135.70 ± 7.78 | This paper |
| Whole Cell from Plasmid | 7.4 ± 1.5 | Rossoni et al. 2015 |
| (MVD) | | |
| Whole Cell from Plasmid | 507 ± 137 | Rossoni et al. 2015 |
| (M3K) | | |

### 3. Discussion

This work is the first demonstration of isobutene production from transgenic expression of M3K and MVD from a central chromosome. Previous bioproduction levels from whole cells with expression from plasmids in E. coli range from 2.57 pmol/min*g cells to 98.13pmol/min*g cells when expressing either wild type scMVD or mutated MVD, respectively. The isobutene production rate of this engineered strain (135.70±7.78 pmol/min*g cells) remains—5 fold below the current highest reported production levels from plasmids (507±137 pmol/min*g cells), but the production strain may improve with further optimization. SS9 is under constitutive expression from the chromosome; thus, it is not expected to have a high gene copy number compared to cells harboring plasmids carrying M3K or MVD, leading to the lower production rate for isobutene. A benefit of expressing genes from the central chromosome is that antibiotic supplementation is not necessary to maintain the genes, which is environmentally and economically advantageous when scaling up. The highest overall bioproduction of isobutene was reported as 2,880±140 pmol/min*mg protein. This was achieved through reactions of purified M3K enzyme with 3-HIV at elevated temperatures (50° C.). Since the reaction of 3-HIV with M3K proceeds through an unstable intermediate that undergoes a spontaneous decarboxylation, it is likely that the increased reaction rate was due to an abundance of free energy for the reaction. Bearing this fact in mind, it might be beneficial to insert M3K and MVD into a thermotolerant microbe to determine if production efficiency could be improved by culturing at higher temperatures.

There have been several thermophilic microbes that have been investigated for their potential as biochemical cell factories. The organism that has demonstrated the most success thus far is Pyrococcus furious. This hyperthermophilic archaea grows optimally at 100° C. and has been engineered previously to increase hydrogen production, or to produce products such as lactate, ethanol, and 3-hydroxypropionate (3-HP). Through a series of engineering steps, 3HP production has been established and increased incrementally to a level 10-fold higher than initial production. One difficulty facing use of P. furiosus is finding non-native enzymes that are thermally stable enough to be functional at temperatures near 100° C. P. torridus, the thermoacidophilic archaeon that M3K was identified in, grows optimally at 60° C. While this temperature is significantly lower than the temperature necessary for the cultivation of P. furiosus, it is more thermostable than other biofuel producing organisms and its enzymes may function in P. furious. Given increased rate of production and available host organisms, it is worth investigating if expressing M3K, since it is more thermally adapted than MVD, in P. furiosus would result in significant production of isobutene.

### C. Solvent Stress Response

There are many challenges associated with biological production of high energy hydrocarbons, one of which is that accumulation of biofuels in cultures can result in toxicity that inhibits further production of the desired fuel. Elucidating the stress response of cell factories following solvent exposure has been demonstrated as an effective method for finding gene targets for engineering solvent tolerant bioproducers. The stress response of the engineered microbe to biologically relevant solvents was investigated through transcriptomic profiling following solvent exposure. Solvents were grouped into two classes of treatments: endogenously produced stressors (acetone, isobutene, and isobutanol), and analogous organic solvents (liquid alkanes, 3-methylpentane, hexane; liquid alkenes 3-methyl-1-pentene, and cyclohexene). The number of significant upregulated genes following treatment ranged from 155 to 254 across treatments, and the number of significant downregulated genes ranged from 185 to 1,214 across treatments (FIG. 3). Solvent induced changes in gene expression were not consistent across both treatments. This is evidenced by the distinct clustering of the two solvent treatment groups, the endogenous solvents and the analogous organic solvents in FIG. 4.

The most commonly upregulated pathways in response to all solvent treatments were heat-shock responses (gene families clp, ibp, hsl) and phage-shock responses (gene family psp; Table 5). FIGS. 5 and 6 show a shared subset of genes that have undergone changes in expression from treatment with endogenous stressors and analogous organic solvents, respectively. All changes in gene expression were generated by testing for changes from the expression levels of the positive control and genes were filtered at a Bon Ferroni Corrected p-value<0.05. When exposed to acetone, isobutanol, or isobutene, 399, 831, and 1384 genes underwent significant changes in expression, respectively (FIG. 3). When exposed to liquid alkane solvents, 535 genes were differentially expressed following exposure to hexane and 414 from 3-methylpentane (FIG. 3). When exposed to the liquid alkene solvents, 530 genes underwent significant changes in expression after treatment with cyclohexene and 540 after exposure with 3-methyl-1-pentene (FIG. 3).

TABLE 5

| | Treatment | | | | | | |
|---|---|---|---|---|---|---|---|
| | | | | | 3- | | 3-methyl-1- |
| Gene | Acetone | Isobutanol | Isobutene | Hexane | methylpentane | Cyclohexene | pentene |
| adiA | 1.50 ± 0.91 | −2.97 ± 0.87* | 0.47 ± 0.86 | −4.64 ± 0.88* | −2.19 ± 0.86* | −1.87 ± 0.86 | −3.34 ± 0.90* |
| adiC | −0.54 ± 1.10 | −2.56 ± 1.03 | −1.83 ± 1.03 | −4.53 ± 1.04* | −1.60 ± 1.02* | −1.64 ± 1.02 | −4.45 ± 1.13* |
| adiY | −0.57 ± 0.71 | −0.17 ± 0.65 | 0.21 ± 0.65 | −3.19 ± 0.66* | −2.88 ± 0.66* | −2.84 ± 0.66* | −4.27 ± 0.72* |
| bhsA | 4.49 ± 0.62* | 4.78 ± 0.60* | 0.43 ± 0.86 | 3.01 ± 0.60* | 2.98 ± 0.60* | 4.18 ± 0.60* | 4.75 ± 0.60* |
| bssR | 2.34 ± .46* | −1.75 ± 1.29 | 3.93 ± 0.44* | 1.50 ± 0.44* | 1.81 ± 0.44* | 2.27 ± 0.44* | −0.03 ± 0.44 |
| bssS | 5.02 ± .88* | 4.08 ± 0.84* | 3.75 ± 0.84* | 5.47 ± 0.84* | 5.42 ± 0.84* | 6.69 ± 0.84* | 5.81 ± 0.84* |
| clpA | 1.92 ± .68* | 2.07 ± 0.63* | 1.73 ± 0.64* | 2.49 ± 0.63* | 2.69 ± 0.63* | 4.12 ± 0.63* | 3.44 ± 0.63* |
| clpB | 4.51 ± .69* | 4.51 ± 0.69* | 2.85 ± 0.69* | 5.02 ± 0.69* | 5.30 ± 0.69* | 6.17 ± 0.69* | 6.13 ± 0.69* |
| clpP | 1.39 ± 0.63 | 0.38 ± 0.59 | 0.09 ± 0.59 | 1.52 ± 0.59 | 1.74 ± 0.59* | 2.72 ± .59* | 2.17 ± 0.59* |
| coaA | −3.10 ± 1.0* | −3.09 ± 0.90* | −4.22 ± 0.95* | −2.34 ± 0.88 | −1.35 ± 0.88 | −1.36 ± 0.88 | −1.75 ± 0.90 |
| coaD | 0.05 ± 1.01 | −1.68 ± 0.97 | −3.52 ± 1.24* | 0.57 ± 0.90 | 1.13 ± 0.90 | 1.34 ± 0.90 | 0.47 ± 0.93 |
| cspA | −4.58 ± 0.64* | −4.88 ± 0.58* | −8.13 ± 0.67* | −4.08 ± 0.57* | −4.58 ± 0.58* | −6.07 ± 0.58* | −4.30 ± 0.58* |
| cspB | −5.17 ± 1.49* | −5.73 ± 1.38* | −9.03 ± 1.74* | −4.25 ± 1.30* | −4.38 ± 1.31* | −4.10 ± 1.31* | −3.27 ± 1.33 |
| cspC | −0.95 ± .98 | −3.36 ± 0.94* | −2.50 ± 0.94* | −2.22 ± 0.90 | −2.37 ± 0.90* | −3.65 ± 0.94* | −1.52 ± 0.93 |
| cspD | 2.52 ± 0.63* | 1.73 ± 0.58* | 3.12 ± 0.58* | 2.07 ± 0.57* | 2.53 ± 0.57* | 2.88 ± 0.57* | 3.11 ± 0.58* |
| cspE | −2.45 ± 0.72* | −1.94 ± 0.64* | −0.85 ± 0.63 | −0.53 ± 0.63 | −0.36 ± 0.63 | −0.06 ± 0.63 | 0.28 ± 0.63 |
| cspF | −5.06 ± 1.34 | −3.40 ± 1.07* | −7.27 ± 1.56* | −3.67 ± 1.04* | −3.22 ± 1.04 | −2.37 ± 1.04 | −2.49 ± 1.08 |
| dhaK | 0.26 ± 0.67 | 1.05 ± 0.59 | 2.25 ± 0.59* | 3.83 ± 0.58* | 4.32 ± 0.58* | 3.72 ± 0.59* | 3.88 ± 0.59* |
| dhaL | −0.41 ± 0.74 | −0.04 ± 0.64 | 0.48 ± 0.65 | 2.77 ± 0.62* | 3.43 ± 0.62* | 2.40 ± 0.62* | 3.17 ± 0.63* |
| dnaJ | 2.96 ± 0.74* | 3.71 ± 0.69* | 0.17 ± 0.70 | 4.46 ± 0.69* | 5.07 ± 0.69* | 6.64 ± 0.69* | 6.43 ± 0.69* |
| dnaK | 4.58 ± 0.66* | 4.53 ± 0.66* | 2.71 ± 0.66* | 4.31 ± 0.66* | 4.55 ± 0.66* | 5.96 ± 0.66* | 4.74 ± 0.66* |
| fabA | −0.85 ± 0.84 | −2.84 ± 0.81* | −2.35 ± 0.81* | −0.96 ± 0.75 | −0.74 ± 0.75 | −1.13 ± 0.76 | −0.23 ± 0.77 |
| fabB | 1.61 ± 0.72 | −0.39 ± 0.68 | −0.08 ± 0.68 | 2.15 ± 0.66* | 2.87 ± 0.66* | 1.01 ± 0.66 | 2.75 ± 0.66* |
| fabD | −1.74 ± 0.59* | −2.33 ± 0.53* | −1.92 ± 0.54* | −0.89 ± 0.52 | −0.97 ± 0.52 | −1.64 ± 0.53* | −0.87 ± 0.53 |
| flgA | −2.14 ± 1.68* | −3.05 ± 1.60 | −3.93 ± 1.72* | −2.91 ± 1.55 | −1.37 ± 1.53 | −0.73 ± 1.53 | −2.55 ± 1.64 |
| flgB | −4.07 ± 1.67* | −8.74 ± 1.90* | −7.15 ± 1.90 | −6.86 ± 1.62* | −3.25 ± 1.50 | −3.31 ± 1.51 | −5.95 ± 1.74 |
| flgC | −4.24 ± 2.06* | −8.87 ± 2.24* | −8.24 ± 2.24 | −7.12 ± 2.02* | −4.33 ± 1.93 | −4.46 ± 1.94 | −5.29 ± 2.06 |
| flgD | −4.39 ± 2* | −6.75 ± 2.03* | −6.54 ± 2.06* | −6.97 ± 1.94* | −3.56 ± 1.87 | −3.72 ± 1.87 | −5.38 ± 2.00* |
| flgE | −4.02 ± 1.30* | −4.68 ± 1.21* | −7.58 ± 1.51* | −5.83 ± 1.20* | −2.72 ± 1.17 | −2.82 ± 1.18 | −4.38 ± 1.24* |
| flgF | −2.98 ± 1.7* | −5.18 ± 1.70* | −7.63 ± 1.96* | −5.93 ± 1.66* | −2.66 ± 1.57 | −2.88 ± 1.58 | −3.45 ± 1.66 |
| flgG | −4.98 ± 1.68* | −6.73 ± 1.69* | −6.35 ± 1.72* | −5.59 ± 1.51* | −3.06 ± 1.48 | −2.77 ± 1.48 | −4.32 ± 1.57* |
| flgH | −2.24 ± 1.82* | −4.17 ± 1.80 | −4.44 ± 1.88* | −4.58 ± 1.75* | −2.28 ± 1.70 | −2.14 ± 1.71 | −3.08 ± 1.80 |
| gabD | 0.73 ± 1.41 | −2.21 ± 1.41 | −0.64 ± 1.36 | −1.81 ± 1.34 | 0.24 ± 1.31 | 0.11 ± 1.32 | −0.39 ± 1.37 |
| gabP | −1.06 ± 1.42 | −1.47 ± 1.32 | −3.65 ± 1.55 | −3.20 ± 1.33 | 1.14 ± 1.28 | 1.19 ± 1.28 | −0.55 ± 1.33 |
| gabT | 0.34 ± 1.38 | −1.57 ± 1.34 | −1.79 ± 1.38 | −1.73 ± 1.30 | 0.62 ± 1.27 | 0.31 ± 1.28 | −0.74 ± 1.35 |
| gadA | 4.57 ± 0.70* | −0.71 ± 0.69 | 1.22 ± 0.68 | −1.47 ± 0.68 | −1.66 ± 0.69 | −0.52 ± 0.68 | −3.97 ± 0.90* |
| gadB | 4.53 ± 0.82* | 0.96 ± 0.79 | 2.70 ± 0.79* | −0.69 ± 0.80 | −1.26 ± 0.80 | 0.17 ± 0.80 | −2.34 ± 0.85* |
| gadC | 4.83 ± 0.82* | 1.56 ± 0.80 | 3.17 ± 0.80* | −0.25 ± 0.80 | −0.31 ± 0.80 | 1.28 ± 0.80 | −1.88 ± 0.83 |
| gadE | 2.23 ± .62* | −0.46 ± 0.60 | 1.75 ± 0.60* | −2.22 ± 0.60* | −2.58 ± 0.60* | −3.51 ± 0.61* | −5.01 ± 0.68* |
| gadW | 1.58 ± 1* | −4.10 ± 0.99* | −4.21 ± 1.02* | −4.92 ± 0.97* | −2.78 ± 0.95* | −2.17 ± 0.95 | −5.73 ± 1.17* |
| gadX | 1.71 ± 0.59* | −2.01 ± 0.57* | −1.69 ± 0.57* | −2.95 ± 0.57* | −2.92 ± 0.57* | −3.69 ± 0.57* | −2.89 ± 0.58* |
| gadY | 0.05 ± 0.83 | −3.37 ± 0.80* | −1.72 ± 0.78 | −5.22 ± 0.81* | −4.93 ± 0.81* | −4.01 ± 0.80* | −4.81 ± 0.90* |
| glpA | 4.83 ± 0.91* | 5.44 ± 0.87* | 9.06 ± 0.87* | 7.69 ± 0.87* | 8.12 ± 0.87* | 7.06 ± 0.87* | 7.04 ± 0.87* |
| glpB | 2.81 ± 0.69* | 2.96 ± 0.64* | 6.10 ± 0.63* | 5.40 ± 0.63* | 5.89 ± 0.63* | 4.64 ± 0.63* | 4.86 ± 0.63* |
| glpC | 3.12 ± 0.87* | 2.92 ± 0.81* | 6.02 ± 0.80* | 6.04 ± 0.79* | 6.69 ± 0.79* | 5.02 ± 0.80* | 5.64 ± 0.80* |
| groL | 4.18 ± 0.58* | 4.05 ± 0.57* | 2.61 ± 0.57* | 4.16 ± 0.57* | 4.27 ± 0.57* | 4.57 ± 0.57* | 4.37 ± 0.57* |
| groS | 4.58 ± 0.51* | 4.43 ± 0.50* | 1.62 ± 0.50* | 4.25 ± 0.50* | 4.18 ± 0.50* | 4.63 ± 0.50* | 4.22 ± 0.50* |
| hchA | 4.64 ± 0.64* | 3.17 ± 0.60* | 3.41 ± 0.60* | 0.77 ± 0.60 | 1.65 ± 0.60* | 2.06 ± 0.60* | 2.03 ± 0.62* |
| hslJ | −0.10 ± 0.75 | 1.12 ± 0.66 | −1.24 ± 0.71 | 2.29 ± 0.65* | 2.40 ± 0.65* | 1.41 ± 0.66 | 1.02 ± 0.67 |
| hslO | 2.78 ± 0.69* | 1.72 ± 0.65* | −0.29 ± 0.66 | 3.04 ± 0.64* | 3.37 ± 0.64* | 4.56 ± 0.64* | 3.42 ± 0.64* |
| hslR | 3.01 ± 0.70* | 2.12 ± 0.65* | −0.04 ± 0.67 | 3.21 ± 0.65* | 3.53 ± 0.65* | 4.80 ± 0.65* | 3.19 ± 0.66* |
| hslU | 2.69 ± 0.59* | 1.52 ± 0.56* | 0.23 ± 0.56 | 3.61 ± 0.55* | 3.65 ± 0.55* | 4.95 ± 0.55* | 3.91 ± 0.56* |
| hslV | 2.24 ± 0.67* | 0.88 ± 0.62 | −1.15 ± 0.66 | 2.94 ± 0.61* | 3.32 ± 0.61* | 4.81 ± 0.61* | 3.66 ± 0.61* |
| ibpA | 5.64 ± 1.22* | 5.74 ± 1.21* | 3.97 ± 1.21* | 7.63 ± 1.21* | 8.01 ± 1.21* | 9.90 ± 1.21* | 9.84 ± 1.21* |
| ibpB | 6.70 ± 1.51* | 6.79 ± 1.50* | 3.99 ± 1.50* | 8.41 ± 1.50* | 9.01 ± 1.50* | 10.95 ± 1.50* | 11.65 ± 1.50* |
| lhgO | 1.82 ± 1.73 | −1.10 ± 1. 73 | −1.00 ± 1.77 | −1.20 ± 1.67 | 1.21 ± 1.64 | 1.38 ± 1.64 | −1.08 ± 1.80 |
| lipA | −1.03 ± 0.69 | 0.31 ± 0.60 | −1.56 ± 0.63* | 0.42 ± 0.59 | 0.26 ± 0.60 | 0.42 ± 0.73 | 0.62 ± 0.61 |
| lipB | −1.11 ± 0.75 | −2.33 ± 0.69* | −4.07 ± 0.88* | −0.05 ± 0.63 | 0.71 ± 0.63 | 0.61 ± 1.03 | 1.26 ± 0.64 |
| narG | 2.69 ± 0.70* | 2.47 ± 0.65* | 4.31 ± 0.65* | 4.03 ± 0.65* | 4.24 ± 0.65* | 4.96 ± 0.65* | 0.67 ± 0.66 |
| narH | 2.94 ± 0.66* | 1.99 ± 0.62* | 3.55 ± 0.61* | 3.23 ± 0.61* | 3.41 ± 0.61* | 4.45 ± 0.61* | −0.40 ± 0.65 |
| narI | 1.48 ± 1.02 | 0.99 ± 0.94 | 1.31 ± 0.94 | 2.02 ± 0.92 | 2.28 ± 0.92 | 3.11 ± 0.92* | −2.41 ± 1.20 |
| narJ | 1.75 ± 0.74 | 1.39 ± 0.69 | 2.32 ± 0.68* | 2.26 ± 0.68* | 2.34 ± 0.68* | 4.11 ± 0.68* | −0.67 ± 0.73 |
| narK | 0.21 ± 0.96 | −0.93 ± 0.90 | −0.07 ± 0.89 | 2.20 ± 0.84 | 2.75 ± 0.84* | 3.31 ± 0.84* | 0.15 ± 0.90 |
| potA | −3.62 ± 0.91* | −5.42 ± 0.87* | −5.02 ± 0.89* | −2.87 ± 0.80* | −2.43 ± 0.80* | −2.79 ± 0.81* | −2.42 ± 0.82* |
| potB | −2.22 ± 1.43 | −2.50 ± 1.32 | −4.42 ± 1.50* | −2.22 ± 1.29 | −0.51 ± 1.28 | −1.05 ± 1.29 | −1.56 ± 1.33 |
| potC | −3.06 ± 1.45 | −2.97 ± 1.28* | −3.91 ± 1.44* | −1.06 ± 1.18 | −0.27 ± 1.18 | −0.55 ± 1.19 | −0.61 ± 1.22 |
| pspA | 2.10 ± 0.60* | 5.48 ± 0.55* | 7.14 ± 0.55* | 8.97 ± 0.55* | 9.23 ± 0.55* | 10.50 ± 0.55* | 10.50 ± 0.55* |
| pspB | 0.76 ± 0.80 | 3.26 ± 0.68* | 4.56 ± 0.68* | 7.11 ± 0.68* | 7.09 ± 0.68* | 9.33 ± 0.68* | 8.15 ± 0.68* |
| rpoA | −0.20 ± 0.47 | −0.58 ± 0.44 | −0.01 ± 0.44 | 0.76 ± 0.44 | 0.54 ± 0.44 | 0.17 ± 0.44 | 0.25 ± 0.44 |
| rpoB | −0.26 ± 0.43 | −0.72 ± 0.40 | −0.34 ± 0.40 | 0.54 ± 0.40 | 0.33 ± 0.40 | 0.79 ± 0.40 | 0.39 ± 0.40 |
| rpoC | −0.29 ± 0.43 | −1.03 ± 0.39* | −0.44 ± 0.39 | 1.02 ± 0.39 | 0.70 ± 0.39 | 1.15 ± 0.39 | 0.99 ± 0.39 |
| rpoD | 1.41 ± 0.72 | 1.30 ± 0.70 | −0.24 ± 0.70 | 3.14 ± 0.70* | 3.38 ± 0.70 | 4.75 ± 0.70* | 3.68 ± 0.70* |
| rpoE | −0.26 ± 0.66 | −1.10 ± 0.62 | −1.87 ± 0.62* | −2.64 ± 0.62* | −2.07 ± 0.62* | 1.44 ± 0.62 | −2.04 ± 0.63* |
| rpoH | 1.72 ± 0.55* | 1.72 ± 0.52* | 0.42 ± 0.52 | 0.31 ± 0.52 | 0.71 ± 0.52 | 1.79 ± 0.52* | 0.68 ± 0.52 |
| rpoN | −1.18 ± 0.54 | −1.50 ± 0.49* | −1.57 ± 0.49* | −1.24 ± 0.49 | −1.28 ± 0.49* | −1.54 ± 0.49* | −1.52 ± 0.50* |
| rpoS | 1.63 ± 0.51* | 1.73 ± 0.47* | 0.24 ± 0.48 | 0.42 ± 0.47 | 0.53 ± 0.47 | 0.22 ± 0.47 | −0.17 ± 0.48 |
| rpoZ | −1.83 ± 0.60* | −0.91 ± 0.54 | −2.12 ± 0.55* | −0.98 ± 0.53 | −1.07 ± 0.53 | −0.02 ± 0.53 | −1.17 ± 0.55 |
| spy | 1.90 ± 0.95 | 8.88 ± 0.87* | 2.48 ± 0.88* | 4.12 ± 0.87* | 4.73 ± 0.87* | 3.95 ± 0.87* | 3.35 ± 0.88* |
| tatA | −1.15 ± 0.57 | −2.04 ± 0.52* | −1.83 ± 0.52* | −0.89 ± 0.51 | −1.05 ± 0.51 | −1.95 ± 0.52* | −1.50 ± 0.52* |
| tatB | −1.35 ± 0.65 | −2.87 ± 0.60* | −2.68 ± 0.61* | −0.66 ± 0.58 | −0.93 ± 0.58 | −1.59 ± 0.59* | −1.06 ± 0.60 |
| tatC | −2.55 ± 1.23 | −4.38 ± 1.21* | −3.61 ± 1.20* | −1.03 ± 1.08 | −0.35 ± 1.08 | −1.25 ± 1.09 | −1.33 ± 1.12 |
| tatD | −1.90 ± 1.23 | −4.11 ± 1.20* | −4.46 ± 1.28* | −2.65 ± 1.13 | −1.73 ± 1.12 | −1.56 ± 1.13 | −2.62 ± 1.19 |
| tatE | 0.41 ± 0.70 | 0.70 ± 0.64 | 0.70 ± 0.64 | 0.78 ± 0.63 | 0.79 ± 0.63 | −0.97 ± 0.64 | −0.18 ± 0.65 |

The quantity of sequence generated for each sample was consistent across most samples, but there were four samples that had diminished sequence returns compared to the other biological replicates in their treatment. The lower-quality data in these samples was due to inexperience with the RNA library preparation protocol during the first attempts. The two most significantly impacted were one control replicate and one acetone treatment replicate (FIG. 4). One sample from each of the cyclohexene and 3-methylpentane samples also generated less sequence data, but not to the degree seen in the acetone and control sample. Statistical analyses were carried out with and without outliers included to determine outlier effects. DESeq2 proved robust in generating models to estimate fold changes with no major differences between the two sets of analyses with or without outliers.

When treated with acetone, isobutanol, or isobutene the strongest upregulation responses were in stress response genes, in particular those involved in protein repair and regulation of intracellular pH and osmolarity. Expression of genes ibpAB and clpAB, involved in protein protection and repair, all increased (FIG. 5). Expression of heat shock proteins and chaperones hslJORUV was mixed, with upregulation in response to acetone and isobutanol treatments but no significant changes in expression for isobutene. Genes encoding chaperone proteins groLS and dnaJK increased expression (Table 5). Genes involved in acid resistance via gamma-aminobutyric acid (GABA) production from glutamate (gadABCE) were upregulated in acetone and isobutene treatments but downregulated in the isobutanol treatment (FIG. 5). Genes involved in acid resistance through conversion of arginine to agmatine adiACY showed mixed responses across treatments but, with the exception of adiA which was downregulated in response to isobutanol, were not statistically significant (FIG. 5). Genes involved in conversion of glycerol to glycerone phosphate (glpABC) increased in expression across all three treatments (Table 5). Genes involved in decreasing production of biofilm material (bssRS) were upregulated across treatments (Table 5). Genes involved in fatty acid biosynthesis (accBC) were downregulated across all three treatments (Table 5). One gene in the multiple antibiotic resistance family marA was upregulated in all three treatments; however, marBC did not pass quality filtering and showed mixed expression (Table 5).

When treated with 3-methyl pentane and hexane, the strongest increases were stress response genes, specifically those involved in protein repair and protection (ibpAB, clpAB, FIG. 5; hslJORUV, Table 5). The phage shock genes pspABCDE were highly upregulated for both treatments (Table 5), as was expression of the chaperone proteins groLS, spy, and dnaJK (Table 5). Genes involved in acid resistance, either through production of GABA from glutamate or conversion of arginine to agmatine, were downregulated in both treatments, but not all members of gad or adi gene families had significant expression changes for both treatments (FIG. 6). Members of the genes encoding proteins for galactitol metabolism gatABD were upregulated (Table 5), whereas gadE and adiY were significantly downregulated (FIG. 5). Glycerol degradation genes glpABC as well as nitrate reductase genes narGHJKP were upregulated across both treatments (Table 5). Genes involved in development of multiple antibiotic resistance phenotypes marABC were upregulated in response to both solvents (Table 5), as were the dihydroxyacetone kinase genes dhaKLM (Table 5). Genes coding for flagellar protein synthesis and assembly were also downregulated in both treatments (Table 5).

When treated with alkene solvents 3-methyl-pentene or cyclohexene, the strongest changes in gene expression were also in stress response genes, specifically those involved in protein repair and protection. Expression of genes coding for protein protection and repair proteins increased across both treatments (ibpAB, clpAB, FIG. 6; hslJORUV, Table 5). Phage shock genes were highly upregulated for both treatments as were chaperone proteins (pspABCDE, groLS, spy, dnaJK, Table 5). Galactitol metabolism (gatABD) was upregulated in both treatments (Table 5). Dihydroxyacetone kinases (dhaKLM) were upregulated in both treatments (Table 5). Nitrate reductase genes narGHJKP were upregulated across both treatments (Table 5). Genes involved in development of multiple antibiotic resistance phenotypes were upregulated in response to both solvents (marABC, Table 5). Genes involved in acid resistance either through production of GABA from glutamate or conversion of arginine to agmatine were downregulated in both treatments. While not all members of gad or adi gene families underwent significant changes in expression for both solvent treatments, gadE and adiY were significant for both treatments (FIG. 6).

### 1. DISCUSSION

i. Transcriptomic Analysis of Solvent Response

Following solvent exposure, there was consistent upregulation of genes involved in heat shock or oxidative stress response. Across all solvent treatments there were upregulation of genes involved in protecting proteins from denaturation and aggregation or in repairing and refolding proteins after damage (clp ibp). These responses act collectively increase protection of proteins from aggregation, refolding damaged proteins, and decreasing membrane fluidity in response to interactions with nonpolar solvents. The strongest effects seen across all solvent treatments were in overexpression of genes for protein protection and repair. This indicates that the strongest negative effects the solvent exposures had were through denaturing proteins. This is important for the production strain in particular since isobutene is gaseous and has very low aqueous solubility. The environment where isobutene has most potential to have deleterious interactions is while it is still held intracellularly, hence the interest in repairing proteins it may have damaged. Since there is work remaining to be done to bring biofuels to a more viable large-scale implementation, it is necessary to design production strains that are resistant to prolonged exposure to these solvents.

There is evidence that improving the tolerance of a production strain can improve production titers, and stress responses following exposure to related compounds such as butanol and isobutanol have been investigated. Isobutanol tolerant mutants were evolved through repeated exposure to isobutanol and subsequent reculturing. In these isobutanol tolerant mutants, they found insertion mutants in gene loci acrA, gatY, marCRAB, rapZ, and tnaA. When the strain engineered for isobutene production was exposed to isobutanol, there were mixed responses in expression of the systems when compared to the study by Atsumi et al 2010. There was upregulation of the gatY and marCRAB loci and downregulation of RNAse adapter protein rapZ (Table 5). In the study by Atsumi et al., they were able to establish isobutanol tolerance in a naive population through deletion of the loci described earlier. Varying degrees of tolerance were conferred through deletion of some or all of the loci. This is important to note, as it demonstrates that not all of the upregulated responses seen in these engineered cells may confer increased tolerance if overexpressed. While numerous studies have shown that selectively overexpressing genes that are highly expressed during stress responses can improve stress tolerance, the Atsumi study shows that one should not blindly assume this to be the case for all upregulated responses. Additionally, it is worth noting that not all strains that have been engineered for enhanced tolerance have shown increased production. There are several other studies however, that have managed to increase product titers for several compounds through increased tolerance in various microbes. Tolerance engineering has been effective for increased production of a number of compounds e.g. butanol, ethanol, 1-3-propanediol, and limonene.

Isobutene is gaseous at room temperature and has an aqueous solubility of 30 mg/L at 20° C. which means it will accumulate primarily in the headspace of the culture vessels. This makes for a system where continual product harvesting will be simple. In the system there is evidence of upregulation of several gene families such as mar and gat that were investigated for isobutanol tolerance. Deletion of some of these highly expressed genes conferred increased tolerance, but did not increase production. Given the expression of gat genes throughout the samples grown on wastewater and their increasing rates of expression at later time points, it indicates deletion of gat genes may prove problematic for the production system in the long term. It will need to be experimentally determined if deletion of gene families such as mar, gat, or arc will increase tolerance to isobutene, as well as to determine the effects of these deletions on the engineered strains' growth rate. The most successful studies at increasing biofuel production through tolerance engineering used overexpression of some of the same stress responses that were seen to be strongly upregulated in the engineered strain. Overexpression of ibpA in an engineered production strain not only conferred increased tolerance but increased isopentenol production by 16%. The strong upregulation of ibpAB in the engineered strain when stressed with isobutene would indicate these are prime candidates to improve production when overexpressed. Once an isobutene tolerant mutant has been engineered, the next steps would be to investigate the effect that this tolerance engineering has on isobutene production. Determining the effects of tolerance engineering on both production rates, and overall isobutene titers will provide insight on how isobutene toxicity may affect isobutene production.

ii. Wastewater Metabolism

When comparing the samples grown with either glucose or wastewater as the carbon source in MOPS minimal media, there were several significant differences in carbon utilization and cell movements. Cultures grown on wastewater show lower expression of genes synthesizing glutamate and conversion of glutamate to GABA than in the glucose cultures. There are high numbers of transcripts coding for enzymes involved in sugar alcohol catabolism in the wastewater cultures. One of the most prevalent families of transcripts in these were gat genes. Galactitol seems to be a primary sugar component of the wastewater as evidenced by strong upregulation of gat genes involved in galactitol uptake and catabolism compared to the glucose cultures. Sugars were not the only carbon component of the wastewater media though, lipid catabolism is upregulated at day 1 compared to glucose samples, but free fatty acids appear to have been depleted in the media by day 3, as genes involved in (3-oxidation were no longer upregulated by day 3. Following cell death phase, anaerobic (3-oxidation genes were upregulated once more likely because of fatty acids available from dead cells in later time points in both culture conditions.

In the samples grown in wastewater supplemented media, there was strong upregulation of genes involved in motility and chemotaxis compared to those grown in glucose. This was most strongly evident in early time point comparisons. Increased motility (fig and fli genes) and chemotaxis (cheAW and tsr genes) are beneficial in the wastewater system due to the media being a fine particulate suspension/solution mixture unlike media supplemented with glucose. The particulate matter harboring carbon is not available for passive uptake; thus cells will need to attach to the surface of a particle and degrade it at the water interface. It is important to note that not all carbon in the wastewater media is in suspension, and the amount of suspended material will decrease as cultures age. This would indicate the chemotactic responses were important for early growth on wastewater but as cultures age will no longer be advantageous. This is supported by increased expression of genes involved in lipopolysaccharide (LPS) production, and biofilm formation as cultures age.

### D. Spaceflight Exposure on Engineered E. Coli

### 1. Introduction

While plastic production on earth has been increasing as time progress, there is one environment populated by humans that does not have current large-scale manufacturing capabilities-space. Astronauts living aboard the ISS represent the furthest flung habitation by humans, as well as the closest to colonization of territory off planet. Absent materials production on ISS, all of the materials used by any of the astronauts in space have to be shipped to location. Shipping is extremely expensive, ranging from $1,000-$10,000 per trip, and is potentially dangerous. Additionally, the need for gas impermeable synthetic rubbers is especially high when working in a vessel where leaks of air could be fatal. Couple this need to the availability of human waste as a microbial feedstock and all of these factors come together to ask the question: What are the effects of exposure to spaceflight on the engineered microbe?

The current need for manufacturing polymers or any other industrially significant material in large quantities aboard the ISS is low, but the benefits of understanding the potential effects of spaceflight exposure on bioproduction is high. There remains a huge amount of work to be done to understand the effects of spaceflight on biological systems in general, not just for biofuel producers. Accordingly, the ISS provides a unique opportunity to investigate the effects of spaceflight exposure on the biofuel production strain. Determining the effects of spaceflight on biofuel producers is a starting point for assembling the knowledge necessary to produce biofuels off planet. There is still an incredible amount of technological investment and advancement needed before humans will be investigating large scale manufacturing outside of Earth; but basic research science, like determining the effects of spaceflight on the bioproduction strain will help future researchers in furthering those technological advancements.

As incredibly well protected and habitable as the ISS is when compared to the vacuum of space, it presents a profoundly different environment than that experienced here on Earth. NASA reports that over the course of a single year here on Earth, humans are exposed to approximately 3 mSv (milliSieverts) of damaging background radiation. In comparison, over a 6-month period an astronaut aboard the ISS would be exposed to 160mSV. This is more than 100× more radiation exposure over the course of a year than down below the protection of the atmosphere. This large dose of high energy radiation will likely influence both gene expression, and the overall genome composition of the engineered strain. Previous work aboard the ISS quantified rates of mutation in microbes such as Bacillus subtilus from exposure to radiation. In this study prolonged exposure to high doses of radiation increased rates of mutation by more than three orders of magnitude compared to controls grown on the ground. This relatively high radiation exposure can also be evidenced by expression of the SOS response system, which causes an increase in the expression of error prone polymerases encoded by dinABD & umuCD.

Previous work has shown varied effects when microbes are exposed to spaceflight, including increased growth rates, biofilm formation, virulence, and secondary metabolite production. This provides some evidence of potential benefits to exposing biofuel producers to spaceflight. Increased biofilm formation would allow for increased generation of biomass in reduced volumes of media. Increased growth rates should lead to increased isobutene production since precursors are produced endogenously rather than scavenged from the environment. Finally, if exposure to spaceflight could be used to increase microbial production of the secondary metabolite 3-HIV, this could further enhance viability of production.

Microgravity is not believed to significantly impact the intracellular dynamics of metabolism. The effects on mass transfer from cells, however, are more marked. It is assumed that given the lack of gravity driven flows, uptake of extracellular nutrients is limited by rate of diffusion. Evidence for the importance of diffusion for nutrient uptake under microgravity is apparent when comparing the size of E. coli cell cultures aboard the ISS to those on Earth. After 49 hours of microgravity exposure, E. coli cells had decreased to 37% of the volume of their counterparts cultured on Earth. This shrinking of cells will shift the surface area to volume ratio of the cells in favor of increasing rates of diffusion, helping them maintain nutrient balance in this challenging environment. Due to this reliance on diffusion in the cultures aboard the ISS, the difference in expression of transcripts coding for porins and transporters to aid in nutrient uptake when compared against ground samples should be detectable.

E. coli sent to space also experiences the stress of freezing. Freezing cells at −80° C. induces expression of a cold shock response. The cold shock effect has been well studied in several system, since the primary method for long-term culture storage is freeze-drying. When cells enter cold shock, they start to repress translation and increase the palmitoleate content in the lipid A layer. Increasing the unsaturation of the fatty acid chains allows for increased membrane fluidity as temperatures decrease. The second response is induction of csp genes for chromosomal maintenance under increased hyper coiling from decreased temperatures. Induction of the SOS repair system is often activated when E. coli cultures are revived from freezing. These transcriptional changes need to be taken into account when determining the effect of spaceflight.

Microgravity conditions have shown to increase and streamline metabolic pathways. The use of microgravity to identify unneeded competing pathways allows for these pathways to be genetically removed and therefore allowing for more efficient isobutene production.

### 2. Effects on Gene Expression

To be able to expand civilization off of earth, there is a need to expand manufacturing off planet as well. Before that very distant concept can be executed, a much more detailed understanding of biological processes in environments outside of earth is needed. Before designing a method to measure microbial isobutene production under microgravity, an understanding of the effects of spaceflight on the engineered strain is needed. These effects were investigated by comparing gene expression data from corresponding time points on the ground and aboard the ISS. For wastewater samples aboard the ISS, the number of significantly dysregulated genes ranged from 1047 to 755, on days one and three respectively (FIG. 7). There was no day 7 ground sample to compare the day 7 ISS sample to, due to sequencing constraints from screening for freezing effects. Extraction of sufficient mRNA for sequencing was not achieved for all replicates of either day 14 or 30 ISS samples grown on wastewater. Ground vs ISS sample comparisons were tested with the glucose cultures for all paired time points (days 1, 3, 14, 30). In ISS samples grown on glucose the strongest changes in gene expression were seen when comparing day 14 (FIG. 7) cultures from ISS to ground. The number of significantly dysregulated genes ranged from 2,216 to 606 on days 1 and 30 respectively (FIG. 7). The strongest changes in gene expression are seen at day 14 with comparable responses when comparing day 1 ground and ISS samples, 2588 significantly expressed genes vs 2216, respectively.

Additionally, the effects of extended growth under starvation conditions were investigated by comparing changes in gene expression over time of ground and ISS samples (FIGS. 8 and 9). Cultures grown on glucose at UAA did not show strong differential expression responses over time comparable to that seen aboard the ISS. Samples were compared first against expression at day 1, then to later time points as well. Very few genes if any for late time point comparisons passed statistical filtering, so those comparisons will not be discussed further. The number of differentially expressed genes ranged from 165 at day 3 after freezing, to 95 genes at day 14.

M3K and MVD transcripts were detected in all samples grown at UAA and aboard the ISS, although not always at consistent abundance. Transcript abundance (1.9%-2.0%) did not significantly change over 30 days in cultures grown at UAA. Transcript abundance was highest among day 1 and day 3 samples and reached 1.98% of transcripts aligning to MVD and 2.2% aligning to M3K for wastewater samples at day 1. After one week, transcript abundance had decreased to 0.88% for M3K and 0.81% for MVD in wastewater samples. In glucose samples at day 1, 1.87% aligned to MVD and 2.11% of sequences aligned to M3K. Transcript abundance decreased over time to their lowest point at day 30. Day 30 glucose samples had alignment rates of 0.93% for M3K and 0.83% for MVD. It must be noted that there is a high degree of sequence homology between M3K and MVD that could influence sequence detection.

To determine the effects that freezing had after 24 hours of incubation, comparisons of the samples, either frozen or not, from ground and ISS were carried out. Day 3 cultures that were never frozen were compared to samples that were frozen for 48 hours at −80° C. at the end of the initial 24-hour incubation then grown again for 48 hours. When comparing the unfrozen day 3 samples from ground vs. the ISS samples, there is a strong upregulation of multiple genes in the cold shock family for both glucose and wastewater cultures (clpABCDEFGHI). The strongest responses that pass statistical filtering are in cspAI for both cultures (Table A-4). When comparing the day 3 ground samples that had been frozen to the ISS samples, only cspA was upregulated in either of the ISS cultures and that response was approximately 3-fold less than it was compared to the unfrozen samples. In both comparisons to determine the effects of freezing, the highest proportion of strong upregulation responses (L2FC>1) were seen when comparing unfrozen samples at day 3 to day 3 ISS samples. The day 30 cultures that had been frozen showed no cold shock responses when compared to either each other or to the ISS samples.

### 3. Wastewater

Changes in gene expression were determined through pairwise testing comparing the control values (day 1) to later time points for ground (days 3, 14, 30) or ISS (days 3, 7) samples. Samples were filtered at Bon Ferroni adjusted p-values <0.05. The number of significantly dysregulated genes in the ground cultures ranged from 779 to 1,841 on days 3 and 14 respectively. In cultures aboard the ISS, the number of genes that underwent significant changes in expression ranged from 1,055 to 1,759 on days 3 and 7 respectively.

The samples sent to the ISS show high levels of transcriptional activity at day 1 compared to ground samples. There is upregulation of ATP synthases, pantothenate kinase, and Tol-Pal genes involved in cell invagination. Day 1 ISS samples showed significant upregulation of many glycolytic genes (gapA, pgi, fbaA, eno, pgk). ISS samples showed high levels of expression of systems involved in motility and chemotaxis compared to ground samples (flg, fli, che). When comparing the ISS and ground samples at day 3 (using the frozen samples to reduce confounding variables) there are increased expression of genes that inhibit translational processes, DNA replication, and initiation stationary phase physiological changes. There was transcriptional evidence of increased biofilm formation and mucoid production and iron storage in ISS cultures grown on wastewater compared to samples grown at UAA.

There was a clear pattern to the grouping of the samples grown aboard the ISS (FIG. 8). All samples were tightly grouped and disperse from the ground samples after day 3. Additionally, the closest grouped sample to ISS day 3 is the ground day 3 sample that was frozen after 24 hours to mimic the ISS sample mistake. days 14 and 30 only have data from ground samples due to the inability to extract sufficient mRNA for sequencing from some replicates of ISS samples. days 14 and 30 ground samples cluster closely together, which is indicative of a change in growth conditions after day 7 that remained relatively stable through day 30. This is supported by upregulation of genes involved in error-prone DNA replication, dipeptide transport, and amino acid degradation that are present in ground samples from both days 14 and 30.

Gene expression was significantly altered throughout the course of growth aboard the ISS. Day 3 samples show upregulation of glp genes indicating accumulation of glycerone phosphate (dihydroxyacetone phosphate) and quinols. In days 3 and 7 there are upregulation of genes involved in acetate catabolism and nitrate reduction (ack, nar). Day 3 and 7 samples show upregulation of genes converting unsaturated fatty acids to saturated fatty acids (cfa), which would decrease membrane fluidity. Glycerol fermentation (glp genes) were upregulated at day 3 but decrease in expression at day 7. There was upregulation of edd and eda genes, indicating the cultures were converting gluconate to G3P. There was upregulation of acid response genes (c/c) but other stress responses are not apparent. By day 14, only two thirds of biological replicates yielded usable quantities of mRNA for sequencing, and only one third day 30 wastewater cultures from the ISS yielded the 5 μg of total RNA needed for input into rRNA depletion to properly balance libraries.

### 4. Glucose

Changes in gene expression over time were determined through pairwise testing of later time points against day 1 samples for ground (days 1, 3, 14, 30) and ISS (days 1, 3, 7, 14, 30) samples respectively. Samples were filtered at Bon Ferroni p-values <0.05. When comparing ISS samples at later time points to day 1 samples, there was a striking difference in the number of significantly differentially expressed genes. The number of significantly expressed genes range from 2,223 to 3,114 genes on days 3 and 14 respectively. One sequencing replicate did not generate comparable amounts of sequence data to the other replicates. This was a glucose day 1 sample that underwent over-drying while going through a bead cleanup and had poor recovery. Analyses were repeated with and without this data point to determine if the outlier had significant impacts on the DESeq-2 results and it did not show significant impacts.

There was a less clear trend to the gene expression data gathered from the samples grown on glucose (FIG. 9). There was tight grouping for days 1, 3, 7, and 30 aboard the space station with day 14 being widely distributed. The clustering of the ground samples was more sporadic with the most interesting groupings being the tight clustering of the day 3 ISS and frozen ground samples and the clustering of all day 14 and 30 ground samples. The clustering of day 3 frozen ground samples with day 3 ISS samples was seen also in the wastewater samples, showing the effect of the accidental freezing on day 3 gene expression. The close grouping of the day 14 and 30 ground samples would indicate that populations have been established and were remaining relatively stable in terms of gene expression. More interesting was the close grouping of days 7 and 30 aboard the ISS with the dispersion of day 14 indicating a shift in gene expression somewhere between days 7 and 14.

Changes in gene expression over time aboard the space station in the glucose samples showed mixed expression of acid stress response genes (gad, adi; FIG. 10, Table 6). Expression of genes involved in protein protection, repair and recycling showed mixed responses in ISS cultures with a general trend of decreasing expression over time (clp, dna, ibp; FIG. 10, Table 6).

After day 3 there was upregulation of several genes involved in anaerobic metabolism, indicating oxygen has been depleted from the environment (glp, pflA, nar; Table 6). From day 14 onward there is expression of genes coding for a wide range of metabolic transporters. By day 14, there was upregulation of genes involved in uptake and degradation of rhamnose (6-deoxy-L-mannose) as well as galactose (rha, gat; Table 6). There was upregulation of uptake and catabolism of amino acids, serine, histidine, ornithine, lysine, arginine, and threonine. The primary metabolic responses that were seen in days 14 and 30 were involved in recycling amino acids, cellular polysaccharides, and fatty acids.

As the cultures aged, there was an increase in transcripts that would likely lead to increased mutations rates. Expression of error-prone polymerases encoded by the increased reaching maximum expression at day 14 (din, umu; FIG. 11, Table 6). Expression of the endogenous CRISPR-Cas system encoded by cas1-3 was upregulated reaching the highest expression at day 14 (FIG. 11, Table 6). In time points after day 7, there was significantly higher expression of numerous insertion sequences (IS) compared to earlier time points aboard the ISS. Additionally, there are numerous insertion sequences that are significantly upregulated when comparing samples aboard the ISS to samples on Earth.

TABLE 6

| | Glucose ISS time Series Tests | | | | |
|---|---|---|---|---|---|
| | Day | Day | Day | Day | Day |
| Gene | 1v3 | 1v7 | 1v14 | 1 v30 | 3v7 |
| aceA | −0.63 ± 0.15* | −0.23 ± 0.13 | −0.46 ± 0.13* | −0.57 ± 0.13* | 0.46 ± 0.13* |
| aceB | −0.94 ± 0.17* | −0.97 ± 0.15* | −0.79 ± 0.15* | −1.10 ± 0.15* | 0.02 ± 0.15 |
| aceE | −2.42 ± 0.20* | −2.84 ± 0.18* | −2.37 ± 0.17* | −3.06 ± 0.18* | −0.49 ± 0.17* |
| aceF | −2.22 ± 0.17* | −2.80 ± 0.15* | −3.07 ± 0.15* | −3.21 ± 0.15* | −0.58 ± 0.15* |
| aceK | 0.06 ± 0.23 | 0.16 ± 0.20 | 0.92 ± 0.20* | 0.21 ± 0.20 | 0.16 ± 0.20 |
| ackA | −2.08 ± 0.18* | −2.43 ± .0.16* | −2.54 ± 0.16* | −2.61 ± 0.16* | −0.33 ± 0.16 |
| adiA | −0.14 ± 0.20 | 0.06 ± 0.18 | 0.62 ± 0.18* | −0.02 ± 0.18 | 0.20 ± 0.18 |
| adiC | 0.19 ± 0.24 | 0.61 ± 0.22* | 1.05 ± 0.21* | 0.66 ± 0.22* | 0.46 ± 0.21 |
| adiY | 0.19 ± 0.17 | 0.35 ± 0.15* | −1.03 ± 0.16* | 0.56 ± 0.15* | 0.21 ± 0.15 |
| bhsA | −0.16 ± 0.24 | 0.21 ± 0.21 | −0.74 ± 0.22* | −0.70 ± 0.22* | 0.28 ± 0.21 |
| bssR | 0.48 ± 0.19* | −0.31 ± 0.17 | −1.46 ± 0.17* | −2.21 ± 0.17* | −0.85 ± 0.17* |
| bssS | 0.42 ± 0.24 | 0.75 ± 0.22* | −0.73 ± 0.22* | −1.04 ± 0.22* | 0.21 ± 0.21 |
| cas1 | 0.37 ± 0.21 | 0.46 ± 0.18* | 0.98 ± 0.18* | 0.39 ± 0.18* | 0.08 ± 0.18 |
| cas2 | 1.03 ± 0.20* | 1.54 ± 0.18* | 1.92 ± 0.18* | 1.70 ± 0.18* | 0.57 ± 0.17* |
| cas3 | 1.00 ± 0.18* | 0.95 ± 0.16* | 1.92 ± 0.16* | 1.10 ± 0.16* | −0.12 ± 0.16 |
| casA | 1.12 ± 0.27* | 0.97 ± 0.24* | 0.60 ± 0.24* | 0.99 ± 0.24* | −0.09 ± 0.24 |
| casB | 1.08 ± 0.33* | 0.69 ± 0.30* | 1.75 ± 0.30* | 0.97 ± 0.30* | −0.45 ± 0.29 |
| casC | 0.67 ± 0.24* | 0.47 ± 0.22* | 0.64 ± 0.22* | 0.69 ± 0.22* | −0.35 ± 0.22 |
| clpA | −1.49 ± 0.21* | −1.94 ± 0.19* | −2.17 ± 0.19* | −2.06 ± 0.19* | −0.54 ± 0.19* |
| clpB | 0.95 ± 0.18* | −0.60 ± 0.16* | −0.62 ± 0.16* | −1.35 ± 0.16* | −1.73 ± 0.18* |
| clpP | −2.10 ± 0.18* | −3.06 ± 0.17* | −2.87 ± 0.16* | −3.43 ± 0.17* | −1.07 ± 0.18* |
| coaA | 0.87 ± 0.19* | 0.94 ± 0.17* | 1.05 ± 0.17* | 1.11 ± 0.17* | 0.06 ± 0.16 |
| coaD | −0.03 ± 0.20* | 0.06 ± 0.18* | −0.19 ± 0.18 | −0.14 ± 0.18 | 0.06 ± 0.18 |
| cspA | −3.97 ± 0.31* | −5.44 ± 0.29* | −6.76 ± 0.29* | −6.09 ± 0.29* | −1.51 ± 0.28* |
| cspB | −3.54 ± 0.23* | −3.59 ± 0.20* | −5.87 ± 0.25* | −3.79 ± 0.20* | −0.02 ± 0.21 |
| cspC | −3.73 ± 0.37* | −5.15 ± 0.37* | −4.31 ± 0.33* | −4.89 ± 0.34* | −1.64 ± 0.39* |
| cspD | −0.42 ± 0.17* | −1.32 ± 0.16* | −0.66 ± 0.15* | −1.45 ± 0.15* | −0.90 ± 0.16* |
| cspE | −2.86 ± 0.25* | −3.79 ± 0.23* | −4.62 ± 0.24* | −3.74 ± 0.22* | −0.97 ± 0.24* |
| cspF | −0.61 ± 0.24* | −0.42 ± 0.21 | −0.45 ± 0.21* | −0.51 ± 0.21* | 0.16 ± 0.21 |
| dhaK | −1.09 ± 0.20* | −1.12 ± 0.18* | −1.24 ± 0.18* | −1.01 ± 0.17* | −0.04 ± 0.19 |
| dhaL | −1.22 ± 0.30* | −1.83 ± 0.28* | −2.00 ± 0.27* | −2.01 ± 0.27* | −0.66 ± 0.29 |
| dinB | 0.13 ± 0.20 | 0.17 ± 0.18 | −0.27 ± 0.18 | 0.48 ± 0.17* | 0.05 ± 0.18 |
| dinD | 0.30 ± 0.15 | 0.23 ± 0.14 | 0.98 ± 0.13* | 0.22 ± 0.13 | −0.06 ± 0.13 |
| dnaJ | 0.45 ± 0.15* | −0.26 ± 0.14 | −0.49 ± 0.14* | −0.61 ± 0.14* | −0.76 ± 0.13* |
| dnaK | 0.37 ± 0.29 | −1.13 ± 0.26* | −1.30 ± 0.26* | −2.07 ± 0.26* | −1.60 ± 0.25* |
| fabA | −1.07 ± 0.20* | −1.81 ± 0.19* | −1.70 ± 0.18* | −1.71 ± 0.18* | −0.81 ± 0.20* |
| fabB | −1.35 ± 0.20* | −1.09 ± 0.18* | −1.67 ± 0.18* | −1.25 ± 0.18* | 0.25 ± 0.18 |
| fabD | −1.76 ± 0.23* | −2.60 ± 0.22* | −3.30 ± 0.22* | −3.12 ± 0.22* | −0.84 ± 0.22* |
| fadD | 0.45 ± 0.17* | 0.32 ± 0.15 | 0.41 ± 0.15* | 0.24 ± 0.15 | −0.20 ± 0.15 |
| fadE | 0.38 ± 0.16* | 0.53 ± 0.14* | 1.15 ± 0.14* | 0.55 ± 0.14 | 0.22 ± 0.14 |
| fadH | 0.05 ± 0.17 | 0.63 ± 0.14* | −0.08 ± 0.15 | 0.70 ± 0.14* | 0.56 ± 0.14* |
| fadI | 0.58 ± 0.22* | 0.42 ± 0.20 | −0.28 ± 0.20 | 0.25 ± 0.19 | −0.17 ± 0.19 |
| fadJ | 0.90 ± 0.18* | 0.67 ± 0.16* | 0.60 ± 0.16* | 0.64 ± 0.16* | −0.25 ± 0.15 |
| flgA | 0.54 ± 0.23 | 0.52 ± 0.21* | 1.11 ± 0.20* | 0.84 ± 0.20* | −0.04 ± 0.20 |
| flgB | 0.14 ± 0.29 | −0.30 ± 0.28 | 0.19 ± 0.25 | −0.16 ± 0.26 | −0.67 ± 0.28 |
| flgC | −0.27 ± 0.44 | −0.35 ± 0.40 | 0.47 ± 0.37 | −0.09 ± 0.38 | −0.21 ± 0.40 |
| flgD | −0.15 ± 0.30 | 0.14 ± 0.26 | 0.37 ± 0.25 | 0.36 ± 0.25 | 0.36 ± 0.26 |
| flgE | −0.02 ± 0.20 | 0.68 ± 0.17* | 0.65 ± 0.17* | 0.86 ± 0.17* | 0.81 ± 0.18* |
| flgF | −0.14 ± 0.32 | 0.19 ± 0.28 | −0.56 ± 0.29 | 0.23 ± 0.27 | 0.28 ± 0.28 |
| flgG | 0.10 ± 0.22 | 0.15 ± 0.20 | 0.39 ± 0.19 | 0.36 ± 0.19 | 0.15 ± 0.20 |
| flgH | 0.97 ± 0.30* | 0.55 ± 0.28 | 0.62 ± 0.27* | 1.06 ± 0.26* | −0.16 ± 0.29 |
| gabD | −0.85 ± 0.26* | −0.88 ± 0.24* | −1.30 ± 0.23* | −0.83 ± 0.22* | 0.04 ± 0.24 |
| gabP | 0.42 ± 0.15* | 0.68 ± 0.14* | 0.47 ± 0.13* | 0.75 ± 0.13* | 0.38 ± 0.14* |
| gabT | −0.41 ± 0.19 | −0.04 ± 0.17 | 0.23 ± 0.16 | −0.11 ± 0.17 | 0.45 ± 0.17* |
| gadA | −0.66 ± 0.20* | −1.46 ± 0.19* | −1.72 ± 0.19* | −1.67 ± 0.18* | −0.77 ± 0.18* |
| gadB | −2.95 ± 0.23* | −3.45 ± 0.21* | −4.96 ± 0.22* | −3.86 ± 0.20* | −0.62 ± 0.21* |
| gadC | −2.29 ± 0.12* | −3.05 ± 0.11* | −2.48 ± 0.11* | −3.06 ± 0.11* | −0.81 ± 0.11* |
| gadE | −1.57 ± 0.29* | −2.14 ± 0.26* | −4.63 ± 0.27* | −3.18 ± 0.26* | −0.66 ± 0.26* |
| gadW | 0.44 ± 0.30 | −0.04 ± 0.27 | −1.19 ± 0.27* | −0.13 ± 0.27 | −0.44 ± 0.26 |
| gadX | −0.02 ± 0.29 | −0.40 ± 0.26 | −0.94 ± 0.26* | −0.51 ± 0.26 | −0.40 ± 0.26 |
| gadY | −1.01 ± 0.21* | −1.72 ± 0.20* | −3.42 ± 0.23* | −1.72 ± 0.19* | −0.77 ± 0.20* |
| gatA | −0.12 ± 0.41 | −0.74 ± 0.38 | −0.92 ± 0.37* | −0.59 ± 0.37 | −0.78 ± 0.37 |
| gatB | −0.77 ± 0.32* | −0.69 ± 0.28* | −1.51 ± 0.29* | −0.36 ± 0.28 | −0.09 ± 0.29 |
| gatD | 0.65 ± 0.20* | 0.64 ± 0.18* | 1.04 ± 0.18* | 0.98 ± 0.18* | −0.08 ± 0.17 |
| gatY | −1.74 ± 0.38* | −2.35 ± 0.35* | −2.15 ± 0.34* | −2.57 ± 0.34* | −0.56 ± 0.35 |
| gatZ | −0.80 ± 0.29* | −1.23 ± 0.26* | −0.71 ± 0.25* | −0.95 ± 0.25* | −0.44 ± 0.25 |
| glpA | 0.60 ± 0.24* | 0.69 ± 0.21* | 0.72 ± 0.21* | 0.68 ± 0.21* | 0.17 ± 0.21 |
| glpB | 0.44 ± 0.20* | 0.53 ± 0.18* | 0.41 ± 0.17* | 0.14 ± 0.17 | 0.02 ± 0.17 |
| glpC | 0.68 ± 0.22* | 0.36 ± 0.21* | 0.62 ± 0.20* | 0.47 ± 0.20* | −0.33 ± 0.19 |
| groL | 0.33 ± 0.15* | −1.00 ± 0.14* | −1.37 ± 0.13* | −1.76 ± 0.14* | −1.51 ± 0.16* |
| groS | 1.34 ± 0.24* | −0.69 ± 0.22* | −2.32 ± 0.23* | −2.52 ± 0.23* | −2.25 ± 0.23* |
| hchA | 0.48 ± 0.23 | 0.65 ± 0.20* | 0.49 ± 0.20* | 0.62 ± 0.20* | 0.09 ± 0.20 |
| hslJ | −1.43 ± 0.36* | −1.90 ± 0.34* | −2.39 ± 0.34* | −2.57 ± 0.34* | −0.35 ± 0.36 |
| hslO | 0.45 ± 0.16* | −0.34 ± 0.15* | 0.63 ± 0.14* | −0.37 ± 0.14* | −0.83 ± 0.14* |
| hslR | 1.22 ± 0.22* | −0.14 ± 0.21 | −1.59 ± 0.23* | −1.46 ± 0.22* | −1.53 ± 0.22* |
| hslU | −0.37 ± 0.19 | −0.87 ± 0.17* | −0.43 ± 0.17* | −0.98 ± 0.17* | −0.50 ± 0.17* |
| hslV | −0.42 ± 0.29 | −1.23 ± 0.27* | −2.04 ± 0.27* | −1.72 ± 0.27* | −0.86 ± 0.26* |
| ibpA | 3.18 ± 0.23* | 3.15 ± 0.21* | 1.77 ± 0.21* | 1.31 ± 0.21* | −0.22 ± 0.21 |
| ibpB | 3.08 ± 0.22* | 2.84 ± 0.20* | 1.53 ± 0.21* | 0.87 ± 0.21* | −0.36 ± 0.19 |
| lhgO | −0.39 ± 0.24 | 0.08 ± 0.21 | −1.05 ± 0.22* | −0.35 ± 0.21 | 0.60 ± 0.22* |
| lipA | −0.94 ± 0.17* | −1.87 ± 0.16* | −1.79 ± 0.16* | −2.09 ± 0.16* | −1.00 ± 0.17* |
| lipB | 2.08 ± 0.14* | 1.57 ± 0.13* | 0.46 ± 0.13* | 0.43 ± 0.13* | −0.65 ± 0.14* |
| narG | 0.41 ± 0.15* | 0.38 ± 0.14* | 0.37 ± 0.14* | 0.26 ± 0.14 | −0.03 ± 0.13 |
| narH | 0.42 ± 0.23 | −0.14 ± 0.21 | 0.17 ± 0.21 | −0.52 ± 0.21* | −0.53 ± 0.21* |
| narI | 0.82 ± 0.19* | 1.11 ± 0.17* | 0.87 ± 0.17* | 0.60 ± 0.17* | 0.31 ± 0.16 |
| narJ | 1.07 ± 0.22* | 0.01 ± 0.21 | −0.60 ± 0.21* | −1.11 ± 0.21* | −1.11 ± 0.19* |
| narK | 0.65 ± 0.18* | 0.65 ± 0.16* | 1.21 ± 0.15* | 0.88 ± 0.16* | 0.07 ± 0.15 |
| potA | 0.31 ± 0.17* | −0.50 ± 0.16* | −0.63 ± 0.15* | −0.63 ± 0.15* | −0.81 ± 0.15* |
| potB | 0.36 ± 0.23* | 0.20 ± 0.20 | 0.92 ± 0.19* | 0.37 ± 0.20 | −0.11 ± 0.20 |
| potC | −0.51 ± 0.32* | −1.68 ± 0.31* | −1.29 ± 0.29* | −1.31 ± 0.29* | −1.15 ± 0.31* |
| pspA | −0.59 ± 0.23* | −0.97 ± 0.20* | −2.02 ± 0.20* | −2.37 ± 0.20* | −0.53 ± 0.21* |
| pspB | −0.53 ± 0.24 | −1.05 ± 0.23* | −0.81 ± 0.22* | −1.64 ± 0.22* | −0.55 ± 0.22 |
| rpoA | −2.39 ± 0.18* | −3.57 ± 0.16* | −3.75 ± 0.16* | −4.09 ± 0.16* | −1.23 ± 0.16* |
| rpoB | −1.06 ± 0.17* | −1.80 ± 0.15* | −1.52 ± 0.15* | −2.09 ± 0.15* | −0.80 ± 0.15* |
| rpoC | −1.34 ± 0.14* | −1.64 ± 0.12* | −1.41 ± 0.12* | −1.88 ± 0.12* | −0.34 ± 0.12* |
| rpoD | −0.35 ± 0.17 | −0.67 ± 0.15* | −1.06 ± 0.15* | −1.54 ± 0.15* | −0.44 ± 0.16* |
| rpoE | 0.73 ± 0.20* | −1.78 ± 0.19* | −1.49 ± 0.18* | −2.96 ± 0.19* | −2.63 ± 0.19* |
| rpoH | 0.13 ± 0.15 | −1.27 ± 0.14* | −1.84 ± 0.14* | −2.40 ± 0.14* | −1.53 ± 0.15* |
| rpoN | −2.00 ± 0.14* | −2.05 ± 0.13* | −1.24 ± 0.12* | −2.27 ± 0.12* | −0.08 ± 0.13 |
| rpoS | −2.75 ± 0.25* | −3.81 ± 0.23* | −4.39 ± 0.23* | −4.32 ± 0.23* | −1.10 ± 0.22* |
| rpoZ | −1.97 ± 0.25* | −2.65 ± 0.23* | −2.67 ± 0.22* | −3.05 ± 0.23* | −0.79 ± 0.24* |
| spy | −0.53 ± 0.20* | −0.62 ± 0.18* | −2.16 ± 0.19* | −1.05 ± 0.18* | −0.13 ± 0.18 |
| tatA | −1.60 ± 0.18* | −2.27 ± 0.17* | −2.33 ± 0.16* | −1.97 ± 0.16* | −0.77 ± 0.18* |
| tatB | −1.84 ± 0.27* | −1.65 ± 0.23* | −1.53 ± 0.23* | −1.86 ± 0.23* | 0.08 ± 0.25 |
| tatC | 0.09 ± 0.19* | 0.18 ± 0.17 | 0.80 ± 0.17* | 0.37 ± 0.17* | 0.11 ± 0.17 |
| tatD | −0.18 ± 0.20* | −0.21 ± 0.18 | −0.49 ± 0.18* | −0.59 ± 0.18* | −0.03 ± 0.18 |
| tatE | −1.14 ± 0.22* | −1.27 ± 0.20* | −2.16 ± 0.20* | −1.56 ± 0.20* | −0.16 ± 0.20 |
| umuC | 0.22 ± 0.29 | 0.39 ± 0.26 | 1.21 ± 0.26* | 0.54 ± 0.26 | 0.26 ± 0.26 |
| umuD | 1.49 ± 0.26* | 1.03 ± 0.24* | 1.99 ± 0.23* | 1.41 ± 0.24* | −0.46 ± 0.22 |

| | Glucose ISS time Series Tests | | | |
|---|---|---|---|---|
| | Day | | Day | Day |
| Gene | 3v14 | Day3v30 | 7v14 | 7v30 |
| aceA | 0.23 ± 0.13 | −0.12 ± 0.13 | −0.23 ± 0.13 | −0.34 ± 0.13 |
| aceB | 0.20 ± 0.15 | −0.11 ± 0.15 | 0.18 ± 0.15 | −0.13 ± 0.15 |
| aceE | −0.02 ± 0.17 | −0.72 ± 0.17* | 0.48 ± 0.17* | −0.22 ± 0.17 |
| aceF | −0.84 ± 0.15* | −0.99 ± 0.15* | −0.27 ± 0.15 | −0.41 ± 0.15 |
| aceK | 0.92 ± 0.20* | 0.21 ± 0.20 | 0.76 ± 0.20* | 0.05 ± 0.20 |
| ackA | −0.44 ± 0.16* | −0.52 ± 0.16* | −0.11 ± 0.16 | −0.19 ± 0.16 |
| adiA | 0.76 ± 0.17* | 0.12 ± 0.17 | 0.56 ± 0.17* | −0.08 ± 0.17 |
| adiC | 0.90 ± 0.21* | 0.51 ± 0.21* | 0.44 ± 0.21 | 0.05 ± 0.21 |
| adiY | −1.17 ± 0.15* | 0.41 ± 0.15* | −1.39 ± 0.15* | 0.20 ± 0.15 |
| bhsA | −0.66 ± 0.22* | −0.63 ± 0.21* | −0.95 ± 0.21* | −0.91 ± 0.21* |
| bssR | −1.26 ± 0.22* | −2.74 ± 0.17* | −1.14 ± 0.17* | −1.90 ± 0.17* |
| bssS | 1.47 ± 0.31* | −1.58 ± 0.22* | −1.47 ± 0.22* | −1.79 ± 0.22* |
| cas1 | 0.60 ± 0.17* | 0.01 ± 0.17 | 0.52 ± 0.17* | −0.07 ± 0.17 |
| cas2 | 0.95 ± 0.17* | 0.73 ± 0.17* | 0.38 ± 0.17* | 0.16 ± 0.17 |
| cas3 | 0.85 ± 0.16* | 0.03 ± 0.16 | 0.97 ± 0.16* | 0.15 ± 0.16 |
| casA | −0.46 ± 0.24 | −0.07 ± 0.23 | −0.37 ± 0.24 | 0.02 ± 0.23 |
| casB | 0.61 ± 0.28 | −0.17 ± 0.28 | 1.07 ± 0.29* | 0.28 ± 0.29 |
| casC | −0.18 ± 0.22 | −0.13 ± 0.22 | 0.18 ± .22 | 0.23 ± 0.22 |
| clpA | −0.77 ± 0.19* | −0.66 ± 0.19* | −0.23 ± 0.19 | −0.12 ± 0.19 |
| clpB | −1.75 ± 0.18* | −2.48 ± 0.18* | −0.03 ± 0.18 | −0.75 ± 0.18* |
| clpP | −0.89 ± 0.17* | −1.45 ± 0.18* | 0.19 ± 0.18 | −0.37 ± 0.19 |
| coaA | 0.16 ± 0.16 | 0.23 ± 0.16 | 0.11 ± 0.16 | 0.17 ± 0.16 |
| coaD | −0.18 ± 0.17 | −0.13 ± 0.17 | −0.24 ± 0.17 | −0.19 ± 0.17 |
| cspA | −2.83 ± 0.29* | −2.17 ± 0.28* | −1.32 ± 0.30* | −0.66 ± 0.29 |
| cspB | −2.30 ± 0.26* | −0.22 ± 0.21 | −2.28 ± .26* | −0.20 ± 0.21 |
| cspC | −0.80 ± 0.35* | −1.37 ± 0.36* | 0.84 ± 0.40* | 0.26 ± 0.41 |
| cspD | −0.24 ± 0.14 | −1.02 ± 0.15* | 0.65 ± 0.15* | −0.13 ± 0.16 |
| cspE | −1.79 ± 0.25* | −0.91 ± 0.23* | −0.82 ± 0.26* | 0.06 ± 0.24 |
| cspF | 0.13 ± 0.21 | 0.08 ± 0.21 | −0.03 ± 0.21* | −0.08 ± 0.21 |
| dhaK | −0.16 ± 0.18 | 0.07 ± 0.18* | −0.17 ± 0.30 | 0.11 ± 0.18 |
| dhaL | −0.84 ± 0.28* | −0.85 ± 0.28 | 0.78 ± 0.10* | −0.18 ± 0.30 |
| dinB | −0.40 ± 0.17* | 0.35 ± 0.17 | −0.44 ± 0.17* | 0.30 ± 0.17 |
| dinD | 0.69 ± 0.12 | −0.06 ± 0.13 | 0.75 ± 0.13* | −0.01 ± 0.13 |
| dnaJ | −0.98 ± 0.13 | −1.11 ± 0.13* | −0.22 ± 0.13 | −0.35 ± 0.13 |
| dnaK | −1.77 ± 0.25* | −2.53 ± 0.25* | −0.17 ± 0.25 | −0.94 ± 0.25* |
| fabA | −0.70 ± 0.18* | −0.71 ± 0.18* | 0.12 ± 0.20 | 0.10 ± 0.20 |
| fabB | −0.33 ± 0.18 | 0.09 ± 0.17 | −0.58 ± 0.18* | −0.15 ± 0.17 |
| fabD | −1.54 ± 0.22* | −1.35 ± 0.22* | −0.69 ± 0.23* | −0.51 ± 0.22 |
| fadD | −0.10 ± 0.14 | −0.28 ± 0.14 | 0.10 ± 0.15 | −0.08 ± 0.15 |
| fadE | 0.83 ± 0.13* | 0.24 ± 0.13 | 0.61 ± 0.13* | 0.02 ± 0.13 |
| fadH | −0.14 ± 0.14 | 0.63 ± 0.13* | −0.70 ± 0.13* | 0.07 ± 0.13 |
| fadI | −0.87 ± 0.19* | −0.34 ± 0.18 | −0.70 ± 0.19* | −0.17 ± 0.18 |
| fadJ | −0.32 ± 0.14 | −0.28 ± 0.14 | −0.07 ± 0.15 | −0.03 ± 0.15 |
| flgA | 0.55 ± 0.18* | 0.28 ± 0.19 | 0.59 ± 0.18* | 0.32 ± 0.19 |
| flgB | −0.18 ± 0.26 | −0.54 ± 0.26 | 0.49 ± 0.27 | 0.13 ± 0.28 |
| flgC | 0.61 ± 0.37 | 0.04 ± 0.38 | 0.83 ± 0.38 | 0.26 ± 0.39 |
| flgD | 0.58 ± 0.25* | 0.57 ± 0.25 | 0.22 ± 0.24 | 0.22 ± 0.24 |
| flgE | 0.78 ± 0.17* | 0.99 ± 0.17* | −0.03 ± 0.17 | 0.18 ± 0.16 |
| flgF | −0.47 ± 0.29 | 0.33 ± 0.27 | −0.75 ± 0.28* | 0.05 ± 0.26 |
| flgG | 0.40 ± 0.19 | 0.36 ± 0.19 | 0.25 ± 0.19 | 0.22 ± 0.19 |
| flgH | −0.09 ± 0.28 | 0.35 ± 0.27 | 0.08 ± 0.28 | 0.52 ± 0.28 |
| gabD | −0.37 ± 0.24 | 0.10 ± 0.23 | −0.41 ± 0.24 | 0.06 ± 0.23 |
| gabP | 0.17 ± 0.14 | 0.45 ± 0.14* | −0.21 ± 0.14 | 0.07 ± 0.14 |
| gabT | 0.73 ± 0.17* | 0.38 ± 0.17* | 0.28 ± 0.16 | −0.06 ± 0.16 |
| gadA | −1.04 ± 0.18* | −0.99 ± 0.18* | −0.27 ± 0.19 | −0.22 ± 0.19 |
| gadB | −2.13 ± 0.23* | −1.03 ± 0.21* | −1.51 ± 0.23* | −0.41 ± 0.22 |
| gadC | −0.24 ± 0.11* | −0.82 ± 0.11* | 0.56 ± 0.11* | −0.02 ± 0.11 |
| gadE | −3.16 ± 0.27* | −1.70 ± 0.26* | −2.50 ± 0.27* | −1.05 ± 0.26* |
| gadW | −1.60 ± 0.27* | −0.53 ± 0.26 | −1.16 ± 0.27* | −0.09 ± 0.26 |
| gadX | −0.95 ± 0.26* | −0.51 ± 0.26 | −0.54 ± 0.26 | −0.11 ± 0.26 |
| gadY | −2.46 ± 0.23* | −0.77 ± 0.19* | −1.70 ± 0.24* | 0.00 ± 0.20 |
| gatA | −0.95 ± 0.37* | −0.62 ± 0.37 | −0.17 ± 0.38 | 0.16 ± 0.37 |
| gatB | −0.90 ± 0.29* | 0.24 ± 0.28 | −0.81 ± 0.29* | 0.33 ± 0.28 |
| gatD | 0.32 ± 0.17 | 0.26 ± 0.17 | 0.41 ± 0.17* | 0.34 ± 0.17 |
| gatY | −0.35 ± 0.34 | −0.78 ± 0.34 | 0.20 ± 0.35 | −0.22 ± 0.35 |
| gatZ | 0.08 ± 0.25 | −0.16 ± 0.25 | 0.52 ± 0.25 | 0.28 ± 0.25 |
| glpA | 0.20 ± 0.20 | 0.16 ± 0.20 | 0.03 ± .20* | −0.01 ± 0.20 |
| glpB | −0.11 ± 0.16 | −0.38 ± 0.17 | −0.12 ± 0.16 | −0.39 ± 0.17 |
| glpC | −0.06 ± 0.18 | −0.21 ± 0.18 | 0.27 ± 0.19 | 0.11 ± 0.19 |
| groL | −1.88 ± 0.16* | −2.27 ± 0.16* | −0.37 ± .16* | −0.76 ± 0.16* |
| groS | −3.88 ± 0.24* | −4.08 ± 0.24* | −1.63 ± 0.24* | −1.83 ± 0.24* |
| hchA | −0.06 ± 0.20 | 0.07 ± 0.20 | −0.16 ± 0.20 | −0.02 ± 0.19* |
| hslJ | −0.84 ± 0.36* | −1.02 ± 0.36* | −0.49 ± .37 | −0.67 ± 0.38 |
| hslO | 0.14 ± 0.13 | −0.86 ± 0.13* | 0.97 ± 0.14* | −0.03 ± 0.14 |
| hslR | −2.98 ± 0.23* | −2.85 ± 0.23* | −1.45 ± 0.24* | −1.32 ± 0.24* |
| hslU | −0.06 ± 0.17 | −0.61 ± 0.17* | 0.44 ± 0.17* | −0.11 ± 0.17 |
| hslV | −1.67 ± 0.27* | −1.35 ± 0.26* | −0.81 ± 0.28* | −0.49 ± 0.27 |
| ibpA | −1.60 ± 0.22* | −2.06 ± 0.22* | −1.38 ± 0.22* | −1.84 ± 0.22* |
| ibpB | −1.67 ± 0.19* | −2.33 ± 0.19* | −1.31 ± 0.19* | −1.97 ± 0.20* |
| lhgO | −0.53 ± 0.23* | 0.17 ± 0.22 | −1.13 ± 0.22* | −0.43 ± 0.21 |
| lipA | −0.92 ± 0.16* | 0.46 ± 0.17* | 0.09 ± 0.17 | −0.21 ± 0.17 |
| lipB | −1.76 ± 0.14* | 0.06 ± 0.16 | −1.11 ± 0.14* | −1.13 ± 0.14* |
| narG | −0.04 ± 0.13 | −0.14 ± 0.13 | −0.01 ± 0.13 | −0.12 ± 0.13 |
| narH | −0.22 ± 0.20 | −0.91 ± 0.20* | 0.31 ± 0.20 | −0.38 ± 0.21 |
| narI | 0.07 ± 0.15 | −0.20 ± 0.16 | −0.24 ± 0.15 | −0.51 ± 0.15 |
| narJ | −1.72 ± 0.19* | −2.23 ± 0.20* | −0.61 ± 0.20* | −1.12 ± 0.21* |
| narK | 0.63 ± 0.15* | 0.30 ± 0.15 | 0.56 ± 0.15* | 0.23 ± 0.15 |
| potA | −0.94 ± 0.14* | −0.94 ± 0.14* | −0.13 ± 0.15 | −0.13 ± 0.15 |
| potB | 0.62 ± 0.19* | 0.07 ± 0.19 | 0.72 ± 0.19* | 0.18 ± 0.19 |
| potC | −0.77 ± 0.29* | −0.79 ± 0.29* | 0.38 ± 0.32 | 0.36 ± 0.32 |
| pspA | −1.58 ± 0.21* | −1.94 ± 0.21* | −1.04 ± 0.21* | −1.40 ± 0.21* |
| pspB | −0.31 ± 0.21 | −1.14 ± 0.22* | 0.24 ± 0.22 | −0.59 ± 0.23 |
| rpoA | −1.41 ± 0.16* | −1.75 ± 0.16* | −0.18 ± 0.16 | −0.52 ± 0.16* |
| rpoB | −0.51 ± 0.15* | −1.09 ± 0.15* | 0.28 ± 0.15 | −0.29 ± 0.15 |
| rpoC | −0.11 ± 0.12* | −0.58 ± 0.12* | 0.23 ± 0.12 | −0.24 ± 0.12 |
| rpoD | −0.83 ± 0.16* | −1.31 ± 0.16* | −0.39 ± 0.16* | −0.87 ± 0.16* |
| rpoE | −2.33 ± 0.18* | −3.80 ± 0.19* | 0.29 ± 0.19* | −1.17 ± 0.19* |
| rpoH | −2.10 ± 0.15* | −2.66 ± 0.15* | −0.57 ± 0.16* | −1.13 ± 0.16* |
| rpoN | 0.73 ± 0.12* | −0.30 ± 0.12 | 0.81 ± 0.12* | −0.22 ± 0.13 |
| rpoS | −1.68 ± 0.22* | −1.62 ± 0.22* | −0.58 ± 0.23* | −0.52 ± 0.23 |
| rpoZ | −0.81 ± 0.23* | −1.20 ± 0.24* | −0.02 ± 0.25 | −0.41 ± 0.25 |
| spy | −1.67 ± 0.19* | −0.56 ± 0.18* | −1.54 ± 0.19* | −0.43 ± 0.18 |
| tatA | −0.83 ± 0.17* | −0.46 ± 0.17* | −0.06 ± 0.18 | 0.31 ± 0.18 |
| tatB | 0.20 ± 0.24 | −0.14 ± 0.24 | 0.12 ± 0.24 | −0.21 ± 0.24 |
| tatC | 0.72 ± 0.16* | 0.29 ± 0.16 | 0.61 ± 0.16* | 0.18 ± 0.16 |
| tatD | −0.31 ± 0.18 | −0.41 ± 0.18 | −0.28 ± 0.18 | −0.38 ± 0.18 |
| tatE | −1.06 ± 0.20* | −0.45 ± 0.20 | −0.90 ± 0.21 | −0.29 ± 0.20 |
| umuC | 1.09 ± 0.26* | 0.42 ± 0.26 | 0.82 ± 0.266 | 0.16 ± 0.26 |
| umuD | 0.49 ± 0.22* | −0.09 ± 0.22 | 0.96 ± 0.22* | 0.38 ± 0.22* |

In both sets of ISS samples, genes involved in growth were unregulated compared to ground samples over the first three days. Upregulation of genes involved in cell division, initiation of translation, ATP synthesis, CoA synthesis, fatty acid synthesis, and protein export was seen at days 1 and 3 but is gone by day 7(Table 7). After seven days, there was an upregulation of proteins dealing with initiation of stationary phase and acid stress response proteins to handle toxic accumulation of fermentation byproducts. Additionally, by day 7 and up through day 30 all samples showed increased expression of amino acid catabolism genes. When comparing the ground samples to the ISS samples, the ISS samples had upregulation of din, ppk and umu genes, which encode error-prone DNA polymerase systems and their regulators (FIG. 11, Table 7). The ISS cultures had decreased expression of ast genes for amino acid transport and catabolism compared to ground samples (Table 7). Since this was the predominant mode of metabolism under extended stationary phase growth this would indicate a reduction in metabolic rate, but an increased rate of mutation. This was also supported by increased expression of the Cas1-3 system in ISS samples compared to ground samples (Table 7).

TABLE 7
Spaceflight Induced Dysregulation of Genes

| | Glucose ISS vs Ground time Series Tests | | | |
|---|---|---|---|---|
| Gene | Day 1 | Day 3 | Day 14 | Day 30 |
| aceA | 0.08 ± 0.39 | 0.13 ± 0.12 | 0.43 ± 0.23 | −0.34 ± 0.35 |
| aceB | 1.18 ± 0.27* | 0.56 ± 0.11* | 0.77 ± 0.22* | 0.29 ± 0.17 |
| aceE | 0.26 ± 0.22 | −1.42 ± 0.30* | −0.73 ± 0.23* | −0.85 ± 0.18* |
| aceF | 0.66 ± 0.36 | −1.96 ± 0.27* | −1.96 ± 0.19* | −1.20 ± 0.31* |
| aceK | −0.49 ± 0.21* | 0.21 ± 0.14 | 1.12 ± 0.27* | −0.12 ± 0.25 |
| ackA | 2.26 ± 0.24* | 0.15 ± 0.20* | −0.18 ± 0.23 | −0.37 ± 0.15 |
| adiA | −0.24 ± 0.23 | 0.22 ± 0.12 | 0.84 ± 0.24* | −0.02 ± 0.20 |
| adiC | −0.26 ± 0.25 | 0.11 ± 0.11 | 0.99 ± 0.27* | 0.38 ± 0.23 |
| adiY | 0.73 ± 0.30* | 0.18 ± 0.17 | −1.16 ± 0.21* | 0.90 ± 0.51 |
| bhsA | −2.30 ± 0.43* | −2.39 ± 0.26* | −2.19 ± 0.30* | −1.08 ± 0.33* |
| bssR | −2.33 ± 0.55* | −1.92 ± 0.37* | −2.96 ± 0.19* | −2.90 ± 0.50* |
| bssS | −1.70 ± 0.56* | −2.12 ± 0.31* | −2.38 ± 0.24* | −1.44 ± 0.49* |
| cas1 | −0.28 ± 0.20 | 0.02 ± 0.14 | 0.52 ± 0.26 | −0.13 ± 0.18 |
| cas2 | −0.04 ± 0.22 | 0.76 ± 0.16* | 1.59 ± 0.21* | 1.19 ± 0.25* |
| cas3 | −0.35 ± 0.17 | 0.66 ± 0.15* | 1.28 ± 0.22* | 0.41 ± 0.20 |
| casA | 0.29 ± 0.33 | 0.55 ± 0.13* | −0.21 ± 0.30 | 0.48 ± 0.43 |
| casB | −0.37 ± 0.30 | 0.81 ± 0.20* | 1.03 ± 0.35* | 0.36 ± 0.26 |
| casC | 0.37 ± 0.26 | 0.67 ± 0.16* | 0.32 ± 0.30 | 0.44 ± 0.31 |
| clpA | −0.64 ± 0.38 | −2.47 ± 0.31* | −2.26 ± 0.19* | −1.58 ± 0.28* |
| clpB | −2.03 ± 0.35* | −1.47 ± 0.43* | −2.33 ± 0.18* | −2.21 ± 0.35* |
| clpP | 1.89 ± 0.29* | −0.91 ± 0.30* | −1.26 ± 0.23* | −1.36 ± 0.25* |
| coaA | 0.76 ± 0.25* | 1.33 ± 0.17* | 1.36 ± 0.24* | 1.38 ± 0.31* |
| coaD | 0.23 ± 0.28 | 0.30 ± 0.21 | 0.19 ± 0.32 | 0.18 ± 0.26 |
| cspA | 6.48 ± 0.48* | 1.60 ± 0.18* | −0.87 ± 0.41 | 0.19 ± 0.61 |
| cspB | 4.87 ± 0.55* | 0.11 ± 0.20 | −2.34 ± 0.41* | −0.03 ± 0.75 |
| cspC | 4.67 ± 0.44* | 0.44 ± 0.30 | −0.01 ± 0.51 | −0.76 ± 0.52 |
| cspD | 0.67 ± 0.30 | 0.38 ± 0.20 | 0.69 ± 0.30 | 0.20 ± 0.25 |
| cspE | 3.92 ± 0.47* | −0.88 ± 0.19* | −1.62 ± 0.37* | −0.23 ± 0.60 |
| cspF | 1.04 ± 0.31* | 0.65 ± 0.19* | 0.64 ± 0.29 | 0.52 ± 0.36 |
| dhaK | 1.01 ± 0.32* | −0.19 ± 0.21 | −0.32 ± 0.27 | 0.03 ± 0.18 |
| dhaL | 0.34 ± 0.30 | −0.58 ± 0.26 | −1.04 ± 0.42* | −0.93 ± 0.33* |
| dinB | −0.61 ± 0.24* | −0.32 ± 0.17 | −0.88 ± 0.29* | −0.03 ± 0.23 |
| dinD | 0.23 ± 0.26 | 0.42 ± 0.13* | 1.13 ± 0.22* | 0.51 ± 0.26 |
| dnaJ | 0.16 ± 0.20 | 0.77 ± 0.32* | −0.36 ± 0.23 | −0.28 ± 0.24 |
| dnaK | −0.89 ± 0.40 | −1.42 ± 0.44* | −2.27 ± 0.30* | −2.22 ± 0.41* |
| fabA | 1.27 ± 0.29* | 0.23 ± 0.18 | −0.36 ± 0.35 | −0.54 ± 0.25 |
| fabB | 1.24 ± 0.23* | −0.19 ± 0.14 | −0.17 ± 0.30 | 0.09 ± 0.19 |
| fabD | 1.91 ± 0.29* | 0.32 ± 0.17 | −1.07 ± 0.30* | −0.67 ± 0.23* |
| fadD | −0.85 ± 0.32* | −0.13 ± 0.14 | −0.18 ± 0.23 | −0.13 ± 0.16 |
| fadE | −1.19 ± 0.25* | −0.19 ± 0.14 | 0.67 ± 0.21* | −0.25 ± 0.31 |
| fadH | −0.85 ± 0.20* | −0.60 ± 0.15* | −0.62 ± 0.23* | 0.32 ± 0.14 |
| fadI | −0.77 ± 0.29* | −0.03 ± 0.21 | −0.88 ± 0.27* | −0.22 ± 0.19 |
| fadJ | −0.66 ± 0.25* | 0.24 ± 0.14 | −0.20 ± 0.25 | −0.08 ± 0.14 |
| flgA | −0.86 ± 0.27* | −0.52 ± 0.21* | 0.23 ± 0.28 | −0.10 ± 0.25 |
| flgB | −0.07 ± 0.34 | −0.17 ± 0.26 | 0.10 ± 0.40 | −0.09 ± 0.28 |
| flgC | −0.42 ± 0.45 | −0.19 ± 0.34 | −0.37 ± 0.46 | −0.89 ± 0.38 |
| flgD | −0.62 ± 0.32 | −0.75 ± 0.24* | −0.04 ± 0.33 | −0.45 ± 0.38 |
| flgE | −0.64 ± 0.23* | −0.36 ± 0.15* | 0.22 ± 0.23 | 0.34 ± 0.16 |
| flgF | −1.01 ± 0.29* | −0.68 ± 0.26* | −1.13 ± 0.46* | −0.56 ± 0.30 |
| flgG | −0.93 ± 0.29* | −0.06 ± 0.19 | 0.29 ± 0.28 | −0.21 ± 0.25 |
| flgH | −1.58 ± 0.28* | −0.58 ± 0.28 | −0.71 ± 0.34 | −0.14 ± 0.24 |
| gabD | −0.61 ± 0.23* | −1.27 ± 0.21* | −1.19 ± 0.38* | −0.69 ± 0.24* |
| gabP | −0.40 ± 0.21 | −0.18 ± 0.15 | −0.18 ± 0.22 | 0.41 ± 0.20 |
| gabT | −0.39 ± 0.35 | −0.46 ± 0.17* | 0.42 ± 0.25 | −0.20 ± 0.32 |
| gadA | 1.84 ± 0.33* | 0.23 ± 0.32 | −0.42 ± 0.30 | −0.22 ± 0.37 |
| gadB | 2.90 ± 0.41* | −0.96 ± 0.40* | −2.47 ± 0.33* | −1.09 ± 0.45 |
| gadC | 1.95 ± 0.26* | −0.67 ± 0.27* | −0.56 ± 0.20* | −0.82 ± 0.20* |
| gadE | 1.51 ± 0.55* | −0.16 ± 0.35 | −2.70 ± 0.31* | −0.29 ± 0.58 |
| gadW | 1.28 ± 0.38* | 1.10 ± 0.14* | −0.48 ± 0.37 | 0.81 ± 0.51 |
| gadX | 1.30 ± 0.40* | 1.31 ± 0.14* | 0.27 ± 0.34 | 1.01 ± 0.40 |
| gadY | 2.59 ± 0.50* | 0.81 ± 0.18* | −1.57 ± 0.39* | 0.51 ± 0.73 |
| gatA | 1.36 ± 0.54* | 1.36 ± 0.43* | −0.20 ± 0.38 | 0.44 ± 0.37 |
| gatB | 1.91 ± 0.48* | 0.25 ± 0.35 | −1.06 ± 0.33* | 0.40 ± 0.44 |
| gatD | −0.03 ± 0.29 | 0.77 ± 0.19* | 0.78 ± 0.21* | 0.48 ± 0.24 |
| gatY | 2.25 ± 0.46* | 0.04 ± 0.31 | −0.16 ± 0.36 | −0.68 ± 0.30 |
| gatZ | 1.18 ± 0.37* | 0.42 ± 0.27 | 0.42 ± 0.23 | −0.01 ± 0.16 |
| glpA | −1.43 ± 0.28* | −0.42 ± 0.21 | −0.37 ± 0.28 | −0.63 ± 0.26 |
| glpB | −0.50 ± 0.30 | −0.40 ± 0.20 | −0.09 ± 0.28 | −0.45 ± 0.19 |
| glpC | −1.35 ± 0.28* | −0.16 ± 0.16 | −0.36 ± 0.26 | −0.56 ± 0.15* |
| groL | 0.06 ± 0.27 | 0.25 ± 0.34 | −0.89 ± 0.20* | −0.71 ± 0.18* |
| groS | −0.12 ± 0.40 | 0.81 ± 0.50 | −2.47 ± 0.23* | −1.63 ± 0.41* |
| hchA | −0.64 ± 0.23* | 0.10 ± 0.15 | −0.05 ± 0.25 | 0.26 ± 0.31 |
| hslJ | 2.32 ± 0.47* | −0.39 ± 0.31 | −1.33 ± 0.52* | −0.79 ± 0.49 |
| hslO | −0.05 ± 0.20 | 0.60 ± 0.18* | 0.95 ± 0.26* | 0.14 ± 0.15 |
| hslR | −0.28 ± 0.34 | 0.05 ± 0.34 | −2.13 ± 0.29* | −1.02 ± 0.40 |
| hslU | −0.04 ± 0.26 | −0.17 ± 0.24 | 0.25 ± 0.26 | −0.41 ± 0.16 |
| hslV | 0.30 ± 0.29 | −0.70 ± 0.32 | −2.15 ± 0.33* | −1.15 ± 0.41* |
| ibpA | −3.75 ± 0.46* | −0.69 ± 0.35 | −1.59 ± 0.22* | −0.42 ± 0.43 |
| ibpB | −3.01 ± 0.53* | −0.76 ± 0.25* | −1.51 ± 0.23* | −0.98 ± 0.40 |
| lhgO | −0.92 ± 0.28* | −0.95 ± 0.22* | −1.30 ± 0.34* | −0.68 ± 0.24* |
| lipA | 0.84 ± 0.24* | 0.49 ± 0.19* | −0.47 ± 0.26 | −0.46 ± 0.22 |
| lipB | −0.26 ± 0.25 | 1.69 ± 0.16* | −0.10 ± 0.22 | 0.21 ± 0.27 |
| narG | −1.53 ± 0.25* | −0.50 ± 0.16* | −0.60 ± 0.19* | −0.63 ± 0.22* |
| narH | −1.56 ± 0.21* | −1.15 ± 0.17* | −0.82 ± 0.24* | −1.18 ± 0.31* |
| narI | −1.37 ± 0.33* | −0.05 ± 0.16 | 0.24 ± 0.27 | −0.45 ± 0.36 |
| narJ | −1.33 ± 0.23* | −0.14 ± 0.39 | −1.39 ± 0.30* | −1.50 ± 0.20* |
| narK | −1.61 ± 0.32* | −0.59 ± 0.13* | −0.11 ± 0.21 | −0.57 ± 0.26 |
| potA | 0.23 ± 0.23 | 0.91 ± 0.14* | −0.23 ± 0.26 | 0.08 ± 0.23 |
| potB | −0.81 ± 0.25* | −0.04 ± 0.19 | 0.50 ± 0.28 | −0.37 ± 0.17 |
| potC | 0.31 ± 0.38 | 0.13 ± 0.25 | −0.58 ± 0.39 | −0.64 ± 0.38 |
| pspA | 0.02 ± 0.47 | −0.24 ± 0.39 | −1.17 ± 0.23* | −1.03 ± 0.31* |
| pspB | 0.39 ± 0.44 | 0.29 ± 0.17 | 0.45 ± 0.36 | −0.57 ± 0.29 |
| rpoA | 3.32 ± 0.31* | 0.52 ± 0.29 | −0.16 ± 0.26 | −0.45 ± 0.20 |
| rpoB | 1.68 ± 0.25* | 0.76 ± 0.23* | 0.69 ± 0.22* | 0.06 ± 0.16 |
| rpoC | 1.16 ± 0.16* | −0.13 ± 0.22 | 0.35 ± 0.19 | −0.06 ± 0.20 |
| rpoD | 0.20 ± 0.21 | −0.36 ± 0.32 | −0.52 ± 0.21* | −0.59 ± 0.18* |
| rpoE | 0.85 ± 0.38 | 0.36 ± 0.33 | −0.89 ± 0.26* | −1.37 ± 0.34* |
| rpoH | −0.75 ± 0.41 | −0.59 ± 0.28 | −1.55 ± 0.22* | −1.22 ± 0.28* |
| rpoN | 1.87 ± 0.18* | 0.10 ± 0.16 | 0.99 ± 0.21* | 0.09 ± 0.15 |
| rpoS | 0.54 ± 0.52 | −2.55 ± 0.30* | −3.33 ± 0.21* | −2.37 ± 0.44* |
| rpoZ | 2.71 ± 0.34* | 0.27 ± 0.20 | −0.24 ± 0.38 | −0.36 ± 0.25 |
| spy | 0.27 ± 0.35 | −0.03 ± 0.21 | −1.33 ± 0.30* | −0.10 ± 0.34* |
| tatA | 1.30 ± 0.38* | 0.82 ± 0.18* | 0.21 ± 0.34 | 1.14 ± 0.36* |
| tatB | 0.92 ± 0.34* | −0.02 ± 0.20 | 0.35 ± 0.36 | −0.27 ± 0.20 |
| tatC | −0.31 ± 0.24 | 0.25 ± 0.16 | 0.77 ± 0.24* | 0.37 ± 0.22 |
| tatD | 0.79 ± 0.32* | −0.13 ± 0.17 | −0.31 ± 0.31 | −0.13 ± 0.22 |
| tatE | 1.12 ± 0.36* | −0.48 ± 0.16* | −1.33 ± 0.30* | −0.10 ± 0.27 |
| umuC | −0.80 ± 0.30* | −0.46 ± 0.17* | 0.65 ± 0.34 | 0.03 ± 0.26 |
| umuD | −0.76 ± 0.38 | 1.22 ± 0.16* | 1.78 ± 0.26* | 0.78 ± 0.24* |

### 5. Discussion

In early time points on the ISS, there is increased transcription of ATP synthetases, pantothenate kinases, genes involved cell invagination and Z ring formation. This is indicative of increased rates of cell division and nutrient cycling in samples aboard the ISS, consistent with trends previously observed. When early time points aboard the ISS are compared to those on the ground, there is upregulation of production of octanoic acid. Fatty acid biosynthesis is also unregulated likely to enhance production of lipoacyl-proteins or lipoacyl-polysaccharides. This would be consistent with reported increases in cellular envelope volume in E. coli cultures exposed to spaceflight. Increased growth rates over the first 72 hours of growth are of particular interest. This time frame corresponds to the highest production rates of isobutene (linear production rate until 48 hours) and the highest levels of M3K and MVD transcript abundance in the first three days of growth in the engineered cultures. These factors support the potential for increased isobutene production under microgravity conditions during exponential growth. Unfortunately, due to shipping weight constraints the effects of spaceflight on isobutene production was not able to be investigated, but the evidence for increased growth rates, metabolic processes, and high transcript levels for M3K and MVD would indicate that isobutene production rates may be elevated.

To determine if spaceflight exposure would have an effect on expression of M3K and MVD, the abundance of transcripts was mapped to each of these proteins over the course of the 30-day experiment aboard the ISS. One main difference stood out between ISS and ground samples. Abundance of transcripts mapping to M3K and MVD in early time points was similar on ground and ISS samples, between 1.9% to 2.2% of total reads mapped. In cultures grown on Earth however, that transcript abundance does not decrease after day 3. At later time points for ground cultures, M3K and MVD transcript alignment rates are still >2.0%, where aboard the ISS, M3K and MVD abundance dropped below 1.0%. M3K and MVD are under constitutive expression, which provokes the question, why is transcript abundance changing over time for these genes, and why is it only happening under certain conditions? One explanation for this could be the decreased expression levels of metabolic genes in ISS samples at days 14 and 30 when compared to ground samples. This may also be partially explained through the gene silencing measures undertaken by the cultures aboard the ISS. When comparing ground and ISS cultures, after day 3, there is upregulation of both type II and type I-e systems aboard the ISS. The type I-e Cascade system is an RNA interference system to knock down foreign transcripts inside a host. This increased expression of interfering RNAs may explain the decrease in transcript abundance for M3K and MVD seen after day 3 in the cultures grown aboard the ISS.

When comparing day 1 samples from ground to ISS, there was an upregulation of general stress response genes rpoS and osmY as well as gadABCDE genes in the ISS samples. There was also strong upregulation of cold shock response genes cspABEF compared to ground samples at day 1. Interestingly, there was also increased transcription of heat shock response genes hslJORUV at day 1 compared to both ground and later ISS samples. Transcription of the general stress response genes is mostly gone by day 3 (when compared to ground samples that underwent freezing) and was also the strongest at day 1 when comparing all later ISS time points to day 1. When comparing day 3 ISS samples, to ground samples that were not frozen at 24 hours, there still was a decrease in the general stress response, but higher expression of csp genes. This expression of both heat and cold shock responses in the day 1 cultures indicates there was more than just one source of stress for the day 1 cultures. Samples were frozen at −80° C. prior to transit and it is possible that they underwent partial thawing and refreezing during transit. This freeze-thaw cycling will induce a stress response of general SOS response systems as well as cold shock adaptations to increase membrane fluidity. The second factor contributing to high expression of stress response genes might be from increased pressure during rocket launch. Previous work in E. coli has shown that when exposed to high pressures, there is increased expression of both heat shock and cold shock genes, as well as SOS response genes. It is important to note however that E. coli has been demonstrated to be active and viable up to gigapascal pressures, which are far higher than anything experienced by the cultures. This would indicate that while increased pressure might induce a stress response it should not reduce viability of the cultures. Finally, after transit cultures were cultured in a high radiation environment, compared to ground controls, which would also induce expression of SOS genes. Seeing expression of stress response systems to help alleviate these different stressors e.g. heat shock proteins, chaperones, polymerases, all indicates that there are several components that together induce a very strong stress response in the day 1 cultures.

There was upregulation of cad genes responsible for conversion of lysine to cadaverine to deal with mixed acid fermentation byproducts after day 3. There was upregulation of genes from day 3 onward involved in polyamine metabolism, primarily with spermidine and putrescine cycling. These genes run from uptake and conversion of arginine to agmatine then conversion of agmatine to putrescine then spermidine, and export/catabolism of spermidine. It appears that this polyamine biosynthesis and neutralization is being used to combat acid stress rather than conversion of glutamate to GABA, as there is downregulation of gad genes at all time points after day 1. Production of GABA is most likely being carried out through metabolism of polyamines. Upregulation of polyamine metabolism after day 3 is most likely due to a need to neutralize nitrogen toxicity from an abundance of amino acids in the media coming from dying cells lysing as cultures age.

After one week, there was evidence of an increase in rhamnose uptake likely from degrading cellular polysaccharides from lysed cells. There was evidence of increased expression of genes involved in various responses to low pH. Upregulation of oxalate catabolism will activate an acid tolerant response (ATR), and increased production of colonic acid for capsule production will also increase tolerance to low pH. As cultures reach the later stages of growth, there was an increase in expression of transcripts coding for genes involved in fatty acid degradation and uptake of monocarboxylic acids, along with some specific amino acids such as serine, lysine, arginine, and threonine. The change from expression of genes involved in metabolism of glucose and byproducts of mixed acid fermentation to catabolism of fatty acids and amino acids indicates that cultures have reached the high rate of cellular turnover found in extended stationary phase cultures. Cultures in this stage of growth have depleted the initial nutrients supplied and are degrading dead cells for nutrients.

From day 3 onward there was upregulation of glp genes to convert glycerol to the glycolytic intermediate glycerone phosphate (DHAP). Under glucose starvation the methylglyoxal pathway is expressed in E. coli as a low energy bypass of the Embden—Meyerhof—Parnas (EMP pathway). There isn't strong evidence of concomitant upregulation of methylglyoxal synthase genes in the cultures. DHAP would be converted to pyruvate through methylglyoxal, but there was not a clear pattern to expression of mgsA. This may be due to the high toxicity of methylglyoxal. Other researchers have shown that methylglyoxal synthase levels in a cell are not accurately reflected in transcript abundance. Additionally, when comparing ground and ISS samples there is upregulation at later times of genes involved further down the glyoxylate cycle, such as aceBK. Taking this information all together, it is likely that the cells are using the methylglyoxal cycle to catabolize G3P and PEP with concomitant excretion of acetate from the cell.

There was upregulation of error-prone polymerases, recombination proteins, and insertion sequences when comparing cultures aboard the ISS to ground cultures. The increase in expression of genes that increase mutation rates was highest in time points after 7 days. This falls in line with previous reporting of mutant phenotypes conferring a growth advantage in stationary phase (GASP) arising in cultures grown for extended periods of time. GASP phenotypes are defined as the ability of mutant cultures to out-compete non-mutant cultures of the same parent strain; i.e., if cells grown for 10+ days are introduced into co-culture with unaged cells of the same strain, after 7-10 days, only previously aged cells will remain. This phenotype is encoded by genetic changes not physiological responses to the environment; this was confirmed by introducing mutations into unaged cell populations that conferred the GASP phenotype onto the naive cells. Four mutations that confer the GASP phenotype have been elucidated and are in the rpoS (alternative sigma factor), lrp (leucine responsive protein), and the gltIJKL cluster (glutamate and aspartate transport). All of these mutations confer an increased ability to break down at least one amino acid as a primary energy source. The increased expression of genes involved in amino acid uptake and catabolism in long term growth cultures lend support to the possibility of GASP mutants being present in the cultures. Amino acid metabolism becomes critically important to extended stationary phase cultures, as the carbon available in the environment is coming from the lysing of other cells. Proteins have been shown to account for up to 55% of cell dry mass in E. coli. This means proteins or peptides will constitute a large portion of the available carbon in the environment after a period of cell death. The increased expression of genes involved in dipeptide and amino acid transport and catabolism in the cultures at and after day 14 indicate that this was the predominant metabolism in the cultures during extended stationary phase.

There were some confounding results in the transcriptomic data, however. There was not consistent detection of changes in expression of sigma factors in the later cultures. This may have been due to mutations in these sigma factors causing mRNA transcripts to not be properly aligned when assembling primary sequencing files to reference genomes. If there were indels in these genes, but they remained functional, sequencing of this mRNA would result in a fragment that would not be assembled into a larger scaffold due to a large gap or mismatch penalty. Mutations in genes that retained functionality might have led to an underestimation of the expression of these genes. Mutation rates from IS insertion is high, approximately 3.5×10⁻⁴ insertions per genome per generation. The high rate of IS driven mutation is evident when you consider a 4 mL culture with OD=1 or approximately 3.2×10⁹ cells, after one generation this culture would have experienced potentially 1.12×10⁶ insertion events. This is in contrast to detection of 95 single nucleotide polymorphisms (SNPs) after more than 650 generations of growth, with only 17 being confirmed not to be sequencing artefacts. Due to the high level of transcription of systems that increase mutation rates, it is reasonable to expect that this will have had some effect on the sequencing. To determine what mutations occurred in the long-term cultures, re-culturing and sequencing can be necessary. Finding mutations in genes that underwent large changes in expression could be an indicator that perhaps that change was an artifact of mutation, rather than a change in expression.

### E. Methods

Described below are the methods used in the Examples described throughout.

### 1. Growth Conditions and Media

All plasmid curation and cloning was carried out with E. coli DH5a, and all recombination and isobutene production was carried out in E. coli K12-MG1655. All cultures grown for plasmid curation were grown with shaking in tryptic soy broth (TSB) supplemented with 0.5% v/v yeast extract (YE) and the appropriate antibiotic at 37° C., with the exception of E. coli DH5a carrying pCas, which was grown at 30° C. Cultures grown for headspace analysis were grown for 24 hours at 37° C. with shaking on MOPS minimal media supplemented with either 1% v/v wastewater or 0.5% w/v glucose.

### 2. Plasmid Construction, PCR, and Cloning

All primers used in this study are listed in Table 8. All plasmid extractions were carried out using Thermo Scientifics' GeneJet Plasmid Miniprep kit according to the manufacturers' protocols. Plasmids pCas (Addgene #62225), pSS9 (Addgene #71655), SS9 RNA (Addgene #71656), and pTargetF (Addgene #62226) were either purchased or received as gifts from the lab of Dr. Ryan Gill. pCas contains the lambda red recombinase genes exo, bet, and gam under arabinose-inducible expression, and Cas9 under constitutive expression. pSS9 contains 600 bp homology arms (H1, H2) that match an intergenic region of the E. coli chromosome that contain a mutation in the protospacer within H1. MVD and M3K plasmids were constructed by cloning the respective genes into Puc57 backbones with ampicillin resistance. A new plasmid pSS9-3KD was constructed through Gibson cloning by removing the GFP and inserting M3K and MVD into pSS9 between the homology arms H1 and H2. A linear fragment containing both M3K and MVD flanked by homology regions was excised by restriction digestion with BglII and Xhol and purified via gel electrophoresis. This fragment was used for recombination experiments and was PCR amplified using a GoTac DNA polymerase under the following conditions for use as transformant DNA: A hot start at 95° C. for 10 minutes, followed by 30 cycles of 95° C. for 30s, 51° C. for 30s, 72° C. for 2.5 minutes, and a final extension at 72° C. for 5 minutes.

TABLE 8
Primers for Transformations and Sequencing

| Primer | Sequence |
|---|---|
| 5′ H1 | ATCCAGCCCACATCGTCC |
| H1 M3K | CGACAGCAACAAGACG |
| F | |
| H1 M3K | AGAATCTGAGCTGCCACTT |
| R | |
| H2 MVD | GTTTGGCTCTGTTCCTGGATG |
| F | |
| H2 MVD | GTGTCCATGGTGTCTGATGAG |
| R | |
| 3′ H2 | TAACCCGCCACAGTAGTTCC |
| ScriptSeq | CAAGCAGAAGACGGCATACGAGATATCACGGT |
| Index 1 | GACTGGAGTTCAGACGTGTGCTCTTCCGATCT |
| ScriptSeq | CAAGCAGAAGACGGCATACGAGATCGATGTGT |
| Index 2 | GACTGGAGTTCAGACGTGTGCTCTTCCGATCT |
| ScriptSeq | CAAGCAGAAGACGGCATACGAGATTTAGGCGT |
| Index 3 | GACTGGAGTTCAGACGTGTGCTCTTCCGATCT |
| ScriptSeq | CAAGCAGAAGACGGCATACGAGATTGACCAGT |
| Index 4 | GACTGGAGTTCAGACGTGTGCTCTTCCGATCT |
| ScriptSeq | CAAGCAGAAGACGGCATACGAGATACAGTGGT |
| Index 5 | GACTGGAGTTCAGACGTGTGCTCTTCCGATCT |
| ScriptSeq | CAAGCAGAAGACGGCATACGAGATGCCAATGT |
| Index 6 | GACTGGAGTTCAGACGTGTGCTCTTCCGATCT |
| ScriptSeq | CAAGCAGAAGACGGCATACGAGATCAGATCGT |
| Index 7 | GACTGGAGTTCAGACGTGTGCTCTTCCGATCT |
| ScriptSeq | CAAGCAGAAGACGGCATACGAGATACTTGAGT |
| Index 8 | GACTGGAGTTCAGACGTGTGCTCTTCCGATCT |
| ScriptSeq | CAAGCAGAAGACGGCATACGAGATGATCAGGT |
| Index 9 | GACTGGAGTTCAGACGTGTGCTCTTCCGATCT |
| ScriptSeq | CAAGCAGAAGACGGCATACGAGATTAGCTTGT |
| Index 10 | GACTGGAGTTCAGACGTGTGCTCTTCCGATCT |
| ScriptSeq | CAAGCAGAAGACGGCATACGAGATGGCTACGT |
| Index 11 | GACTGGAGTTCAGACGTGTGCTCTTCCGATCT |
| ScriptSeq | CAAGCAGAAGACGGCATACGAGATCTTGTAGT |
| Index 12 | GACTGGAGTTCAGACGTGTGCTCTTCCGATCT |

### 3. Lambda Red Mediated Recombination

E. coli K12-MG1655 were transformed with pCas through electroporation with a Bio-Rad MicroPulser. The transformed strain was grown at 30° C. in 250 mL TSB with kanamycin to an OD600˜0.4-0.6, at which point expression of lambda red recombinases was induced via addition of 10 mM L-arabinose. Following induction, samples were grown for an additional two hours at 30° C. and cotransformed with the SS9 RNA plasmid and the PCR amplified linear fragment via electroporation. Transformants were recovered in 37° C. LB with shaking for eight hours and then plated on LB+ tetracycline and kanamycin. Transformants were selected from colonies and grown at 37° C. overnight to cure pCas, and successful gene integration was confirmed by detection of isobutene in culture headspace via GC-MS.

### 4. Wastewater Sample Preparation

Wastewater samples (2L) were taken by pumping effluent from the secondary effluent tank at the John M. Asplund Wastewater Treatment Facility in Anchorage, AK in September of 2018. Wastewater samples were sterilized via a heterothermic sequence of pasteurization (80° C. for 3 hours) followed by cooling (to room temperature), freezing (−20° C.) and thawing. The sequence was carried out four times, for a total of 12 hours of heating at 80° C., and four freeze-thaw cycles. Pasteurization was performed in lieu of autoclaving because autoclaving has been shown to significantly change the abiotic properties of wastewater. Subsamples (50 mL) were homogenized to break apart large particulate matter (bead beading; 5 minutes×5), added to MOPS minimal media at 10 mL/L (1% v/v) and stored at 4° C.

### 5. Detection and Quantification of Isobutene

All detection and quantification of isobutene was carried out via gas chromatography. All sample were grown in 20 mL Restek headspace vials and processed using a headspace autosampler coupled to an Agilent 6890N GC-FID with a Restek Rxi 624sil-ms 30 m×0.25 mm ID×1.4 μm dF column. Analytical standards were diluted from a reference standard of isobutene (AirLiquide). Samples were grown at 37° C. at 200 RPM shaking, in 3.9 mL of MOPS minimal media+1% wastewater supplemented with 50 mM 3-HIV. Overnight cultures of the recombinant E. coli were grown in TSB+0.5% YE at 37° C. with shaking (220 rpm). Cultures were pelleted (5,000xg), supernatant removed, cells resuspended in 5 μL of nuclease free H₂O by vortexing, and cells (100 mg) added to each culture vessel as inoculum. Samples were also grown under the same conditions above but were filtered through a 0.22 μm filter, and cell masses recorded. Samples containing only 4 mL MOPS+1% wastewater and 50 mM 3-HIV were also analyzed as a control to measure spontaneous isobutene production from 3-HIV decomposition. GC-HS conditions were: headspace oven: 80° C., loop: 90° C., transfer line: 100° C., oven equilibration time: 10 minutes, injection: 1 minute. GC oven initial: 35° C., hold for 1.5 minutes, ramp at 30° C./minute to 80° C., ramp at 6° C./minute to 116° C., ramp at 120° C./minute to 300° C., hold 2 minutes. Helium was used as the carrier gas at a rate of 7.1 mL/minute. The flame ionization detector (FID) was set at 300° C. and the fuel gas flow (H2) was set to 40 mL/minute, air flow was set to 450 mL/minute, and the makeup gas (N2) was set to 45 mL/minute.

### 6. Solvent Tolerance Transcriptomics

Engineered strains of E. coli were exposed to solvents at various concentrations to assess the effects of solvent exposure on gene expression. The solvents used were as follows: acetone 1% v/v, isobutanol 1% v/v, 3-methyl-1-pentene 0.75% v/v, 3-methylpentane 0.75% v/v, hexane 0.75% v/v, and cyclohexene 0.5% v/v. Isobutene is gaseous at room temperature, so media was prepared through sparging isobutene through TSB media in a sealed vessel for 3 minutes, which was then used to grow the cultures. Cells were grown in TSB supplemented with solvent at 37° C. with shaking (220 RPM) until cultures reached OD 0.6 when 4×10⁸ cells were harvested for RNA extraction. Samples were pelleted and resuspended in 1 mL RNALater prior to extraction of RNA using Qiagen RNEasy mini kit. Following RNA extraction, samples were subjected to on-column DNase digestion followed by Ribo-Zero rRNA depletion before library prep.

### 7. Spaceflight Transcriptomics

Samples were sent to the ISS to determine the effects of spaceflight on the engineered strain. 5 mL universal cryo-tubes were used as growth vessels with 4 mL of either MOPS minimal media+0.5% glucose, or MOPS minimal media+1% wastewater. Cells for inoculum were grown in TSB with 0.5% YE at 37° C. with shaking overnight (220 RPM). The overnight culture was pelleted at 5,000 X g, the supernatant removed, the pellet resuspended in 10 μL of filter sterilized 50% glycerol via vortexing, and each vessel was inoculated with resuspended cells (1004). After inoculation the media was vortexed and frozen at −80° C. for transport. Samples were made in triplicate to be grown over a 30-day period. Subsets of samples were grown for 1 day, 3 days, 7 days, 14 days and 30 days. A corresponding set of samples was prepared and grown under the same conditions in the lab to use as a control for differential gene expression resulting in a total of 30 vessels (2×3 vessels per time point) inoculated and sent to the space station, and 30 inoculated and grown in the lab. Growth conditions were 25° C. with no shaking. All RNA extractions were carried out as described above.

### 8. Accounting for Freezing Effects

Due to human error aboard the ISS, additional ground samples were required. At the end of the 24 hour time point, all vessels aboard the ISS were removed from the incubator and frozen at −80° C. At the end of the 72-hour timepoint, the researcher on the ISS realized their mistake, and the 3, 7, 14, and 30-day samples were removed from the freezer and put back in the incubator to finish incubating. To account for the effects of freezing, two more sets of triplicate samples were prepped at UAA as stated above and grown for 24 hours. After 24 hours, the samples were frozen for 48 hours and placed back at 25° C. to thaw. One set of samples was incubated for 48 more hours (equivalent to day 3 sample from ISS) and the other set were grown an additional 29 days to compare to the day 30 ISS samples.

### 9. Library Preparations

Libraries were prepared following the Illumina Script-Seq V2 RNA library prep guide to produce 500 bp cDNA libraries. Each library had 12 individual RNA samples indexed with the Script-Seq primers (Table 8) for demultiplexing. Pooled libraries were quantified via qPCR following Kappa's library quantification protocol. Each solvent exposure library was diluted to 4 nM with a 5% PhiX spike-in and each ISS comparison library used a 1% PhiX spike-in. The libraries were sequenced on an Illumina MiSeq, using a V3 600 cycle kit to generate 250 bp paired-end reads.

### 10. RNA-Seq Analysis

All fastq files were sorted into read pairs then merged as single fastq files. These file were trimmed using TRIMMOMATIC with a sliding window of length 5, and quality score of 30. Minimum length for files was set to 75 bp. FastQC was used to check sequences after trimming for adapter contamination, or low sequence quality. Bowtie2 was used to build indices and align the fastq files. The alignment was performed in end to end mode, with the minimum quality scores (S) calculated as per Equation 3, where “L” is the length of the fragment being aligned. Penalty scores were set to mismatch: −6, gap open: −3, gap extension: −3.

S=(−0.6)+(−0.6*L) (3)

Files were run as unpaired reads, since they had been concatenated into single read files, and the SAM files generated were used to count genes and assign genome coordinates. Feature counts were carried out in HTSeq and features were assigned to reads by defining a set: S(i) composed of all features overlapping at position i in a read. If the union of all sets S(i) contains only one item then the read is assigned to that feature. If the union contains more than one feature the reads are designated ambiguous and not assigned as counts for the feature. Additionally, any reads that aligned to multiple features were excluded and designated as non-unique alignments. The final constraint applied to the feature count assignment was a minimum alignment quality score of 10. The alignment quality score (Q) is calculated by Bowtie2 through Equation 4, where p is an estimation of the probability that the calculated alignment is not correctly assigned to the true location of the read.

Q=−10*log 10(p) (4)

### 11. Differential Gene Expression Analysis Using DESeq-2

The feature count files generated from HTSeq-count were reformatted to import into DESeq-2 as DESeqDataSets grouped by treatment conditions (i.e., solvent exposure, spaceflight exposure, freezing, and length of incubations). Count matrices were fitted to a Generalized Linear Model (GLM) in the form of a negative binomial distribution with mean and dispersion values calculated from size factors for the mean and intra-group variability for dispersion factors. Fold changes are estimated by generating maximum likelihood estimates (MLE) from the GLM fits described above. The coefficients describing logarithmic fold changes (LFC) are then fitted to a normal distribution (centered at 0) to the distribution of MLEs over all the genes in the count table. The distribution generated here is used as a priori for a second set of GLM fitting and a maximum a priori is used as the final value for the reported LFC. For the analyses, log base two was used for transformation so values are reported as Log₂ Fold Change (L2FC). This method of estimating LFCs helps correct for large absolute LFC values in lowly expressed genes being falsely detected as strong interactions. This final distribution is also used to calculate the standard error used in the Walds' test for differential expression. In the Walds' test, the LFC estimate is divided by its' standard error term. This calculation yields the z-statistic, which is compared back against a normal distribution. The P-values generated from this test are then passed through an independent filtering procedure based on estimating false discovery rates (FDR). The genes that pass this filter are then adjusted via the Benjamini and Hochberg method for multiple testing.

### 12. Expression Networks

Cytoscape was used to generate expression networks from the transcriptomic data. Gene tables were uploaded and annotated with ontology functions using the Gene Ontology Resource (GO). For differential expression datasets, genes uploaded were filtered at adjusted p-values <0.05 and the networks generated were then filtered at p-values of 0.01. The genes were then color-coded by the strength of their log 2-fold-change for ease of interpretation.

### F. Conclusions

In summary, the engineered strain of E. coli can produce isobutene from expression of M3K and MVD from the central chromosome under constitutive expression. Following exposure to solvent stress, there was a conserved response of increased expression of heat shock genes (ibpAB, hslJOR, and clpABP) to help protect and repair protein damage. There was also elevated expression of genes encoding chaperones groELS and dnaJK which play supporting roles in the process mentioned above. Following solvent exposure, there was an increase in expression of gad genes, coding for enzymes that convert glutamate to GABA to deal with excess intracellular protons for acetone, and isobutene, two of the most relevant solvents, but a decrease in expression for all other solvents. Additionally, there was downregulation of the adiACY genes involved in conversion of arginine to agmatine to deal with low pH for almost all solvent treatments. When compared to the long-term stress responses of the 30-day ISS cultures however, there is upregulation of ibpAB and adiAC genes in cultures grown past 14 days, but downregulation of clpABP and gadABCDE. This indicates that solvent stress from isobutene production might not be as strong of an effect as environmental stress from the accumulation of secondary metabolites. Since there was a downregulation of glutamate conversion in the long-term growth samples but an upregulation in the cultures exposed to high concentrations of isobutene, it might improve tolerance of the culture to supplement the media with additional glutamate. Overall this study has resulted in the design of a production strain to generate isobutene from a low-cost feedstock. This study has also elucidated several avenues for genetic optimization from transcriptomic sequencing of various stress responses in the engineered strain.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Gourmelon G. Global Plastic Production Rises, Recycling Lags. :7.
2. Geyer R, Jambeck J R, Law K L. Production, use, and fate of all plastics ever made. Science Advances. 2017;3:e1700782.
3. Narancic T, O'Connor K E. Microbial biotechnology addressing the plastic waste disaster. Microbial Biotechnology. 2017; 10:1232-5.
4. Szklo A, Schaeffer R. Fuel specification, energy consumption and CO₂ emission in oil refineries. Energy. 2007; 32:1075-92.
5. Centi G, Quadrelli E A, Perathoner S. Catalysis for CO₂ conversion: a key technology for rapid introduction of renewable energy in the value chain of chemical industries. Energy and Environmental Science. 2010; 4:1166-9.
6. Schietekat C, Cauwenberge D, Van Geem K, Marin G. Computational Fluid Dynamics-Based Design of Finned Steam Cracking Reactors. American Institute of Chemical Engineers. 2013; 7:405-10.
7. Davis S J, Caldeira K, Matthews H D. Future CO₂ Emissions and Climate Change from Existing Energy Infrastructure. 2010; 5317.
8. Van Leeuwen BNM, Van Der Wulp A M, Duijnstee I, Van Maris AJA, Straathof AJJ. Fermentative production of isobutene. Applied Microbiology and Biotechnology. 2012; 93:1377-87.
9. Lynch S, Eckert C, Yu J, Gill R, Maness P-C. Overcoming substrate limitations for improved production of ethylene in E. coli. Biotechnology for Biofuels. BioMed Central; 2016; 9:1-10.
10. Guo M, Song W, Buhain J. Bioenergy and biofuels: History, status, and perspective. Renewable and Sustainable Energy Reviews. 2015; 42:712-25.
11. Campbell-Platt G. Fermented foods—a world perspective. Food Research International. 1994; 27:253-7.
12. Ladygina N, Dedyukhina E G, Vainshtein M B. A review on microbial synthesis of hydrocarbons. Process Biochemistry. 2006; 41:1001-1014.
13. Antoni D, Zverlov V V, Schwarz W H. Biofuels from microbes. Appl Microbiol Biotechnol. 2007; 77:23-35.
14. Hawkins A B, Lian H, Zeldes B M, Loder A J, Lipscomb G L, Schut G J, et al. Bioprocessing analysis of Pyrococcus furiosus strains engineered for CO₂-based 3-hydroxypropionate production. Biotechnology and Bioengineering. 2015; 112:1533-43.
15. Liu L, Zhu Y, Li J, Wang M, Lee P, Du G, et al. Microbial production of propionic acid from propionibacteria: Current state, challenges and perspectives. Critical Reviews in Biotechnology. 2012; 32:374-81.
16. Saxena R K, Anand P, Saran S, Isar J, Agarwal L. Microbial production and applications of 1,2-propanediol. Indian J Microbiol. 2010; 50:2-11.
17. Edwards M C, Henriksen E D, Yomano L P, Gardner B C, Sharma L N, Ingram L O, et al. Addition of Genes for Cellobiase and Pectinolytic Activity in Escherichia coli for Fuel Ethanol Production from Pectin-Rich Lignocellulosic Biomass. Appl Environ Microbiol. 2011; 77:5184-91.
18. Choi Y J, Lee S Y. Microbial production of short-chain alkanes. Nature. 2013; 502:571-4.
19. Schirmer A, Rude M A, Li X, Popova E, Cardayre S B del. Microbial Biosynthesis of Alkanes. Science. 2010; 329:559-62.
20. Lynch S, Eckert C, Yu J, Gill R, Maness P-C. Overcoming substrate limitations for improved production of ethylene in E. coli. Biotechnology for Biofuels. 2016; 9:1-10.
21. Wilson J, Gering S, Pinard J, Lucas R, Briggs B R. Bio-production of gaseous alkenes: ethylene, isoprene, isobutene. Biotechnology for Biofuels. 2018; 11:234.
22. Nagahama K, Ogawa T, Fujii T, Fukuda H. Classification of ethylene-producing bacteria in terms of biosynthetic pathways to ethylene. Journal of Fermentation and Bioengineering. 1992; 73:1-5.
23. Van Leeuwen BNM, Van Der Wulp A M, Duijnstee I, Van Maris AJA, Straathof AJJ. Fermentative production of isobutene. Applied Microbiology and Biotechnology. 2012; 93:1377-1387.
24. Gogerty D S, Bobik T A. Formation of isobutene from 3-hydroxy-3-methylbutyrate by diphosphomevalonate decarboxylase. Applied and Environmental Microbiology. 2010; 76:8004-8010.
25. Alvarez T M, Paiva J H, Ruiz D M, Cairo JPLF, Pereira T O, Paix??o DAA, et al. Structure and function of a novel cellulase 5 from sugarcane soil metagenome. PLoS ONE. 2013; 8:1-9.
26. Alvarez T M, Goldbeck R, dos Santos C R, Paix??o DAA, Gon??alves TA, Franco Cairo JPL, et al. Development and Biotechnological Application of a Novel Endoxylanase Family GH10 Identified from Sugarcane Soil Metagenome. PLoS ONE. 2013;8.
27. Buermans HPJ, Den Dunnen J T. Next generation sequencing technology: Advances and applications. Biochimica et biophysica acta. 2014; 1842:1932-1941.
28. Bentley F K, Melis A. Diffusion-based process for carbon dioxide uptake and isoprene emission in gaseous/aqueous two-phase photobioreactors by photosynthetic microorganisms. Biotechnology and Bioengineering. 2012;
29. Johnson E. New Biofuel debut: biopropane. Biofuels, Bioproducts and Biorefining. 2012; 6:246-56.
30. Guo X, Liu J, Xiao B. Bioelectrochemical enhancement of hydrogen and methane production from the anaerobic digestion of sewage sludge in single-chamber membrane-free microbial electrolysis cells. International Journal of Hydrogen Energy. Elsevier Ltd; 2013; 38:1342-7.
31. Reisch C R, Prather K U. The no-SCAR (Scarless Cas9 Assisted Recombineering) system for genome editing in Escherichia coli. Scientific reports. Nature Publishing Group; 2015; 5:15096.
32. Bassalo M C, Garst A D, Halweg-Edwards A L, Grau W C, Domaille D W, Mutalik V K, et al. Rapid and Efficient One-Step Metabolic Pathway Integration in E. coli. ACS Synthetic Biology. 2016; acssynbio.5b00187.
33. Steen E J, Kang Y, Bokinsky G, Hu Z, Schirmer A, McClure A, et al. Microbial production of fatty-acid-derived fuels and chemicals from plant biomass. Nature. Nature Publishing Group; 2010; 463:559-62.
34. Zhang F, Rodriguez S, Keasling J D. Metabolic engineering of microbial pathways for advanced biofuels production. Current Opinion in Biotechnology. Elsevier Ltd; 2011; 22:775-83.
35. Gerbrandt K, Chu P L, Simmonds A, Mullins K A, MacLean H L, Griffin W M, et al. Life cycle assessment of lignocellulosic ethanol: A review of key factors and methods affecting calculated GHG emissions and energy use. Current Opinion in Biotechnology. Elsevier Ltd; 2016; 38:63-70.
36. Popp J, Lakner Z, Harangi-Rakos M, Fan M. The effect of bioenergy expansion: Food, energy, and environment. Renewable and Sustainable Energy Reviews. 2014; 32:559-78.
37. Podola B, Li T, Melkonian M. Porous Substrate Bioreactors: A Paradigm Shift in Microalgal Biotechnology? Trends in Biotechnology. 2017; 35:121-132.
38. Liu B, Benning C. Lipid metabolism in microalgae distinguishes itself. Current Opinion in Biotechnology. 2013; 24:300-309.
39. Converti A, Casazza A A, Ortiz E Y, Perego P, Del Borghi M. Effect of temperature and nitrogen concentration on the growth and lipid content of Nannochloropsis oculata and Chlorella vulgaris for biodiesel production. Chemical Engineering and Processing: Process Intensification. 2009; 48:1146-1151.
40. Sun X-M, Ren L-J, Zhao Q-Y, Ji X-J, Huang H. Microalgae for the production of lipid and carotenoids: a review with focus on stress regulation and adaptation. Biotechnology for Biofuels. 2018; 11:272.
41. Olkiewicz M, Tones C M, Jim??nez L, Font J, Bengoa C. Scale-up and economic analysis of biodiesel production from municipal primary sewage sludge. Bioresource Technology. 2016; 214:122-131.
42. Ditzig J, Liu H, Logan B E. Production of hydrogen from domestic wastewater using a bioelectrochemically assisted microbial reactor (BEAMR). International Journal of Hydrogen Energy. 2007; 32:2296-304.
43. De Clercq D, Wen Z, Fan F, Caicedo L. Biomethane production potential from restaurant food waste in megacities and project level-bottlenecks: A case study in Beijing. Renewable and Sustainable Energy Reviews. 2016; 59:1676-1685.
44. Wagner R C, Regan J M, Oh S-E, Zuo Y, Logan B E. Hydrogen and methane production from swine wastewater using microbial electrolysis cells. Water Research. 2009; 43:1480-8.
45. Ogejo J A, Li L. Enhancing biomethane production from flush dairy manure with turkey processing wastewater. Applied Energy. 2010; 87:3171-7.
46. Woertz I, Feffer A, Lundquist T, Nelson Y. Algae Grown on Dairy and Municipal Wastewater for Simultaneous Nutrient Removal and Lipid Production for Biofuel Feedstock. Journal of Environmental Engineering. 2009; 135:1115-1122.
47. Olkiewicz M, Plechkova N V, Fabregat A, Stither F, Fortuny A, Font J, et al. Efficient extraction of lipids from primary sewage sludge using ionic liquids for biodiesel production [Internet]. Elsevier B. V.; 2015. Available from: http://dx.doi.org/10.1016/j.seppur.2015.08.038
48. Ward A, Ball A, Lewis D. Halophytic microalgae as a feedstock for anaerobic digestion. Algal Research. 2015; 7:16-23.
49. Huffer S, Roche C M, Blanch H W, Clark D S. Escherichia coli for biofuel production: Bridging the gap from promise to practice. Trends in Biotechnology. Elsevier Ltd; 2012; 30:538-45.
50. Beasley J E, Planes F J, Rezola A, Pey J, Tobalina L. Advances in network-based metabolic pathway analysis and gene expression data integration. 2014;16.
51. Lee S, J. Mitchell R. Perspectives on the use of transcriptomics to advance biofuels. AIMS Bioengineering. 2015; 2:487-506.
52. Kleijntjens R H. Bioreactors. 2000; 14:329-47.
53. Wei\s s S, Tauber M, Somitsch W, Meincke R, Müller H, Berg G, et al. Enhancement of biogas production by addition of hemicellulolytic bacteria immobilised on activated zeolite. Water Research. 2010; 44:1970-1980.
54. Granata T. Dependency of Microalgal Production on Biomass and the Relationship to Yield and Bioreactor Scale-up for Biofuels: a Statistical Analysis of 60+ Years of Algal Bioreactor Data. Bioenergy Research. 2016;1-21.
55. Foo J L, Jensen H M, Dahl R H. Improving Microbial Biogasoline Production in Escherichia coli Using Tolerance Engineering. 2014; 5:1-9.
56. Rau M H, Calero P, Lennen R M, Long K S, Nielsen A T. Genome-wide Escherichia coli stress response and improved tolerance towards industrially relevant chemicals. Microbial Cell Factories. 2016; 15:176.
57. Dunlop M J, Dossani Z Y, Szmidt H L, Chu H C, Lee T S, Keasling JD, et al. Engineering microbial biofuel tolerance and export using efflux pumps. Mol Syst Biol. 2011; 7:487.
58. Mukhopadhyay A. Tolerance engineering in bacteria for the production of advanced biofuels and chemicals. Trends in Microbiology. 2015; 23:498-508.
59. Fisher M A, Boyarskiy S, Yamada M R, Kong N, Bauer S, Tullman-Ercek D. Enhancing Tolerance to Short-Chain Alcohols by Engineering the Escherichia coli AcrB Efflux Pump to Secrete the Non-native Substrate n-Butanol. ACS Synth Biol. 2014; 3:30-40.
60. Chen B, Ling H, Chang M W. Transporter engineering for improved tolerance against alkane biofuels in Saccharomyces cerevisiae. Biotechnology for Biofuels. 2013; 6:21.
61. Tomas C A, Welker N E, Papoutsakis E T. Overexpression of groESL in Clostridium acetobutylicum Results in Increased Solvent Production and Tolerance, Prolonged Metabolism, and Changes in the Cell's Transcriptional Program. Appl Environ Microbiol. 2003; 69:4951-65.
62. Fukuda H, Fujii T, Sukita E, Tazaki M, Nagahama S, Ogawa T. Reconstitution of the isobutene-forming reaction catalyzed by cytochrome P450 and P450 reductase from Rhodotorula minuta: decarboxylation with the formation of isobutene. Biochem Biophys Res Commun. 1994; 201:516-22.
63. Fujii T, Ogawa T, Fukuda H. Preparation of a Cell-Free, Isobutene-Forming System from Rhodotorula minuta. Appl Environ Microbiol. 1988; 54:583-4.
64. Allard M, Anissimova M, Marliere P. Methods for producing isobutene from 3-methylcrotonic acid [Internet]. 2017 [cited 2019 Jun 8]. Available from: https://patents.google.com/patent/WO2017085167A2/en
65. Rossoni L, Hall S J, Eastham G, Licence P, Stephens G. The putative mevalonate diphosphate decarboxylase from Picrophilus torridus is in reality a mevalonate-3-kinase with high potential for bioproduction of isobutene. Applied and Environmental Microbiology. 2015; 81:2625-34.
66. Kim W, Tengra F K, Young Z, Shong J, Marchand N, Chan H K, et al. Spaceflight Promotes Biofilm Formation by Pseudomonas aeruginosa. PLOS ONE. 2013;8:e62437.
67. Arunasri K, Adil M, Charan K V, Suvro C, Reddy S H, Shivaji S. Effect of Simulated Microgravity on E. coli K12 MG1655 Growth and Gene Expression. PLOS ONE. 2013;8:e57860.
68. Aunins T R, Erickson K E, Prasad N, Levy S E, Jones A, Shrestha S, et al. Spaceflight Modifies Escherichia coli Gene Expression in Response to Antibiotic Exposure and Reveals Role of Oxidative Stress Response. Front Microbiol [Internet]. 2018 [cited 2019 Mar 10];9. Available from: https://www.ncbi.nlm.nih.gov/pmc/articles/PMC5865062/
69. Moeller R, Reitz G, Nicholson the PT Wayne L, Horneck G. Mutagenesis in Bacterial Spores Exposed to Space and Simulated Martian Conditions: Data from the EXPOSE-E Spaceflight Experiment PROTECT. Astrobiology. 2012; 12:457-68.
70. Rabbow E, Horneck G, Rettberg P, Schott J-U, Panitz C, L′Afflitto A, et al. EXPOSE, an Astrobiological Exposure Facility on the International Space Station—from Proposal to Flight. Orig Life Evol Biosph. 2009; 39:581-98.
71. Janion C. Inducible SOS Response System of DNA Repair and Mutagenesis in Escherichia coli. Int J Biol Sci. 2008; 4:338-44.
72. Little J W, Mount D W. The SOS regulatory system of Escherichia coli. Cell. 1982; 29:11-22.
73. Gao H, Liu Z, Zhang L. Secondary metabolism in simulated microgravity and space flight. Protein Cell. 2011; 2:858-61.
74. Venkateswaran K, Duc MTL, Horneck G. Microbial Existence in Controlled Habitats and Their Resistance to Space Conditions. Microbes Environ. 2014;ME14032.
75. Zea L, Prasad N, Levy S E, Stodieck L, Jones A, Shrestha S, et al. A Molecular Genetic Basis Explaining Altered Bacterial Behavior in Space. PLOS ONE. 2016;11:e0164359.
76. Huang B, Li D-G, Huang Y, Liu C-T. Effects of spaceflight and simulated microgravity on microbial growth and secondary metabolism. Mil Med Res [Internet]. 2018 [cited 2019 Mar 10];5. Available from: https://www.ncbi.nlm.nih.gov/pmc/articles/PMC5971428/
77. Kim H W, Matin A, Rhee M S. Microgravity Alters the Physiological Characteristics of Escherichia coli O₁₅₇:H7 ATCC 35150, ATCC 43889, and ATCC 43895 under Different Nutrient Conditions. Appl Environ Microbiol. 2014; 80:2270-8.
78. Zea L, Larsen M, Estante F, Qvortrup K, Moeller R, Dias de Oliveira S, et al. Phenotypic Changes Exhibited by E. coli Cultured in Space. Front Microbiol [Internet]. 2017 [cited 2019 May 25]; 8. Available from: https://www.frontiersin.org/articles/10.3389/fmicb.2017.01598/full
79. Phadtare S, Inouye M. The Cold Shock Response. EcoSal Plus [Internet]. 2008 [cited 2019 Apr 1];3. Available from: http://europepmc.org/abstract/med/26443733
80. Recent developments in bacterial cold-shock response.—PubMed—NCBI [Internet]. [cited 2019 Apr 1]. Available from: https://www.ncbi.nlm.nih.gov/pubmed/15119823
81. Rosen R, Buchinger S, Pfander R, Pedhazur R, Reifferscheid G, Belkin S. SOS gene induction and possible mutagenic effects of freeze-drying in Escherichia coli and Salmonella typhimurium. Appl Microbiol Biotechnol. 2016; 100:9255-64.
82. Kandror O, DeLeon A, Goldberg A L. Trehalose synthesis is induced upon exposure of Escherichia coli to cold and is essential for viability at low temperatures. PNAS. 2002; 99:9727-32.
83. Rodriguez-Vargas S, Estruch F, Randez-Gil F. Gene Expression Analysis of Cold and Freeze Stress in Baker's Yeast. Applied and Environmental Microbiology. 2002; 68:3024-30.
84. Barria C, Malecki M, Arraiano C M. Bacterial adaptation to cold. Microbiology. 2013; 159:2437-43.
85. Jones P H, Prasad D. The Effect of Sterilization Techniques on Wastewater Properties. Journal (Water Pollution Control Federation). 1968;40:R477-83.
86. Bolger A M, Lohse M, Usadel B. Trimmomatic: A flexible trimmer for Illumina sequence data. Bioinformatics. 2014; 30:2114-2120.
87. Langmead B, Salzberg S L. Fast gapped-read alignment with Bowtie 2. Nature Methods. 2012; 9:357-9.
88. Anders S, Pyl P T, Huber W. HTSeq-a Python framework to work with high-throughput sequencing data. Bioinformatics. 2015; 31:166-9.
89. Love M I, Huber W, Anders S. Moderated estimation of fold change and dispersion for RNA-seq data with DESeq2. Genome Biol. 2014; 15:550.
90. Benjamini Y, Hochberg Y. Controlling the False Discovery Rate: A Practical and Powerful Approach to Multiple Testing. Journal of the Royal Statistical Society Series B (Methodological). 1995; 57:289-300.
91. Cline M, Smoot M, Cerami E, Kuchinsky A, Landys N, Workman C, et al. Integration of biological networks and gene expression data using Cytoscape. Nature protocols. 2007; 2:2366-2382.
92. Ashburner M, Ball C A, Blake J A, Botstein D, Butler H, Cherry J M, et al. Gene Ontology: tool for the unification of biology. Nat Genet. 2000; 25:25-9.
93. The Gene Ontology Resource: 20 years and still Going strong. Nucleic Acids Res. 2019;47:D330-8.
94. Atsumi S, Wu T-Y, Machado IMP, Huang W-C, Chen P-Y, Pellegrini M, et al. Evolution, genomic analysis, and reconstruction of isobutanol tolerance in Escherichia coli. Molecular Systems Biology [Internet]. 2010 [cited 2019 Mar 7];6. Available from: http://msb.embopress.org/cgi/doi/10.1038/msb.2010.98
95. Fraley C D, Kim J H, McCann M P, Matin A. The Escherichia coli Starvation GenecstC Is Involved in Amino Acid Catabolism. Journal of Bacteriology. 1998; 180:4287-90.
96. Baquero F, Martinez J-L, Canton R. Antibiotics and antibiotic resistance in water environments. Current Opinion in Biotechnology. 2008; 19:260-5.
97. Rizzo L, Manaia C, Merlin C, Schwartz T, Dagot C, Ploy MC, et al. Urban wastewater treatment plants as hotspots for antibiotic resistant bacteria and genes spread into the environment: A review. Science of The Total Environment. 2013; 447:345-60.
98. Basen M, Sun J, Adams MWW. Engineering a Hyperthermophilic Archaeon for Temperature-Dependent Product Formation. Giovannoni S J, editor. mBio [Internet]. 2012 [cited 2019 Mar 21];3. Available from: https://mbio.asm.org/lookup/doi/10.1128/mBio.00053-12
99. Nguyen TAD, Han S J, Kim J P, Kim M S, Sim S J. Hydrogen production of the hyperthermophilic eubacterium, Thermotoga neapolitana under N₂ sparging condition. Bioresource Technology. 2010.
100. Zeldes B M, Keller M W, Loder A J, Straub C T, Adams MWW, Kelly R M. Extremely thermophilic microorganisms as metabolic engineering platforms for production of fuels and industrial chemicals. Front Microbiol [Internet]. 2015 [cited 2019 Mar 21];6. Available from: https://www.frontiersin.org/articles/10.3389/fmicb.2015.01209/full
101. Lipscomb G L. Deletion of acetyl-CoA synthetases I and II increases production of 3-hydroxypropionate by the metabolically-engineered hyperthermophile Pyrococcus furiosus Elsevier Enhanced Reader [Internet]. [cited 2019 Mar 21]. Available from: https://reader.elsevier.com/reader/sd/pii/S1096717613001316?token=A3648545F2174A7085FD 2258A38F713292A9D0D264EC1470069D78E7D4D5DD5CC46E3380285148C3DCF12DE87 67DF339
102. Schut G J, Nixon W J, Lipscomb G L, Scott R A, Adams M. Mutational Analyses of the Enzymes Involved in the Metabolism of Hydrogen by the Hyperthermophilic Archaeon Pyrococcus furiosus. Front Microbiol [Internet]. 2012 [cited 2019 Mar 21];3. Available from: https://www.frontiersin.org/articles/10.3389/fmicb.2012.00163/full
103. Zeldes B M, Keller M W, Loder A J, Straub C T, Adams MWW, Kelly R M. Extremely thermophilic microorganisms as metabolic engineering platforms for production of fuels and industrial chemicals. Front Microbiol [Internet]. 2015 [cited 2019 Mar 21];6. Available from: https://www.ncbi.nlm.nih.gov/pmc/articles/PMC4633485/
104. Angelov A, Fütterer 0, Valerius 0, Braus G H, Liebl W. Properties of the recombinant glucose/galactose dehydrogenase from the extreme thermoacidophile, Picrophilus torridus. The FEBS Journal. 2005; 272:1054-62.
105. Fütterer 0, Angelov A, Liesegang H, Gottschalk G, Schleper C, Schepers B, et al. Genome sequence of Picrophilus torridus and its implications for life around pH 0. Proceedings of the National Academy of Sciences. 2004; 101:9091-6.
106. Brynildsen M P, Liao J C. An integrated network approach identifies the isobutanol response network of Escherichia coli. Molecular Systems Biology. 2009; 5:277.
107. Reyes L H, Almario M P, Kao K C. Genomic library screens for genes involved in n-butanol tolerance in Escherichia coli. PLoS ONE. 2011; 6:e17678.
108. Cornet M, Rogiers V. Metabolism and toxicity of 2-methylpropene (isobutene)—A review. Critical Reviews in Toxicology. 1997; 27:223-232.
109. Pubchem. Isobutene [Internet]. [cited 2019 Mar 10]. Available from: https://pubchem.ncbi.nlm.nih.gov/compound/8255
110. Dunlop M J. Engineering microbes for tolerance to next-generation biofuels. Biotechnol Biofuels. 2011; 4:32.
111. Baer S H, Blaschek H P, Smith T L. Effect of Butanol Challenge and Temperature on Lipid Composition and Membrane Fluidity of Butanol-Tolerant Clostridium acetobutylicum. Applied and environmental microbiology. 1987; 53:2854-61.
112. Chong H, Yeow J, Wang I, Song H, Jiang R. Improving Acetate Tolerance of Escherichia coli by Rewiring Its Global Regulator cAMP Receptor Protein (CRP). PLoS ONE. 2013;8.
113. Chong H, Huang L, Yeow J, Wang I, Zhang H, Song H, et al. Improving Ethanol Tolerance of Escherichia coli by Rewiring Its Global Regulator cAMP Receptor Protein (CRP). PLoS One [Internet]. 2013 [cited 2019 Mar 11];8. Available from: https://www.ncbi.nlm.nih.gov/pmc/articles/PMC3585226/
114. Burk M J. Sustainable production of industrial chemicals from sugars. INTERNATIONAL SUGAR JOURNAL. 2010; 112:6.
115. Kacena M A, Merrell G A, Manfredi B, Smith E E, Klaus D M, Todd P. Bacterial growth in space flight: logistic growth curve parameters for Escherichia coli and Bacillus subtilis. Applied Microbiology and Biotechnology. 1999; 51:229-34.
116. Roy B, Zhao J, Yang C, Luo W, Xiong T, Li Y, et al. CRISPR/Cascade 9-Mediated Genome Editing-Challenges and Opportunities. Front Genet [Internet]. 2018 [cited 2019 Mar 22];9. Available from: https://www.ncbi.nlm.nih.gov/pmc/articles/PMC6042012/
117. Cooper L A, Stringer A M, Wade J T. Determining the Specificity of Cascade Binding, Interference, and Primed Adaptation In Vivo in the Escherichia coli Type I-E CRISPR-Cas System. mBio. 2018;9:e02100-17.
118. Jones P G, Inouye M. The cold-shock response—a hot topic. Molecular Microbiology. 1994; 11:811-8.
119. Ishii A, Oshima T, Sato T, Nakasone K, Mori H, Kato C. Analysis of hydrostatic pressure effects on transcription in Escherichia coli by DNA microarray procedure. Extremophiles. 2005; 9:65-73.
120. Marietou A, Nguyen ATT, Allen E E, Bartlett D H. Adaptive laboratory evolution of Escherichia coli K-12 MG1655 for growth at high hydrostatic pressure. Front Microbiol [Internet]. 2015 [cited 2019 May 27];5. Available from: https://www.frontiersin.org/articles/10.3389/fmicb.2014.00749/full
121. Zhao F, Bi X, Hao Y, Liao X. Induction of Viable but Nonculturable Escherichia coli O157:H7 by High Pressure CO2 and Its Characteristics. PLOS ONE. 2013;8:e62388.
122. Sharma A, Scott J H, Cody G D, Fogel M L, Hazen R M, Hemley R J, et al. Microbial Activity at Gigapascal Pressures. Science. 2002; 295:1514-6.
123. Ohnishi K, Ohnishi T. The Biological Effects of Space Radiation during Long Stays in Space. Biol Sci Space. 2004; 18:201-5.
124. Takahashi A, Ohnishi K, Yokota A, Kumagai T, Nakano T, Ohnishi T. Mutation Frequency of Plasmid DNA and Escherichia coli Following Long-term Space Flight on Mir. J Radiat Res. 2002;43: S137-40.
125. Shah P, Swiatlo E. A multifaceted role for polyamines in bacterial pathogens. Molecular Microbiology. 2008; 68:4-16.
126. Casero R A, Pegg A E. Spermidine/spermine N1-acetyltransferase—the turning point in polyamine metabolism. The FASEB Journal. 1993; 7:653-61.
127. Feehily C, Karatzas K a. G. Role of glutamate metabolism in bacterial responses towards acid and other stresses. Journal of Applied Microbiology. 2013; 114:11-24.
128. Samsonova N N, Smirnov S V, Altman I B, Ptitsyn L R. Molecular cloning and characterization of Escherichia coli K12 ygjG gene. BMC Microbiology. 2003;10.
129. Reitzer L. Nitrogen Assimilation and Global Regulation in Escherichia coli. Annual Review of Microbiology. 2003; 57:155-76.
130. Rhee H J, Kim E-J, Lee J K. Physiological polyamines: simple primordial stress molecules. Journal of Cellular and Molecular Medicine. 2007; 11:685-703.
131. Fontenot E M, Ezelle K E, Gabreski L N, Giglio E R, McAfee J M, Mills A C, et al. YfdW and YfdU are required for oxalate-induced acid tolerance in Escherichia coli K-12. J Bacteriol. 2013; 195:1446-55.
132. Reid A N, Whitfield C. Functional Analysis of Conserved Gene Products Involved in Assembly of Escherichia coli Capsules and Exopolysaccharides: Evidence for Molecular Recognition between Wza and Wzc for Colanic Acid Biosynthesis. Journal of Bacteriology.
133. β-Lactam induction of colanic acid gene expression in Escherichia coli FEMS Microbiology Letters Oxford Academic [Internet]. [cited 2019 Mar 10]. Available from: https://academic.oup.com/femsle/article/226/2/245/578008
134. Pletnev P, Osterman I, Sergiev P, Bogdanov A, Dontsova O. Survival guide: Escherichia coli in the stationary phase. Acta Naturae. 2015; 7:22-33.
135. Navarro Llorens J M, Tormo A, Martinez-Garcia E. Stationary phase in gram-negative bacteria. FEMS Microbiol Rev. 2010; 34:476-95.
136. Weber J, Kayser A, Rinas U. Metabolic flux analysis of Escherichia coli in glucose-limited continuous culture. II. Dynamic response to famine and feast, activation of the methylglyoxal pathway and oscillatory behaviour. Microbiology (Reading, Engl). 2005; 151:707-16.
137. Layton J C, Foster P L. Error-prone DNA polymerase IV is controlled by the stress-response sigma factor, RpoS, in Escherichia coli. Molecular Microbiology. 2003; 50:549-61.
138. Tang M, Shen X, Frank E G, O'Donnell M, Woodgate R, Goodman M F. UmuD′2C is an error-prone DNA polymerase, Escherichia coli pol V. PNAS. 1999; 96:8919-24.
139. Tompkins J D, Nelson J L, Hazel J C, Leugers S L, Stumpf J D, Foster P L. Error-Prone Polymerase, DNA Polymerase IV, Is Responsible for Transient Hypermutation during Adaptive Mutation in Escherichia coli. Journal of Bacteriology. 2003; 185:3469-72.
140. Lee H, Doak T G, Popodi E, Foster P L, Tang H. Insertion sequence-caused large-scale rearrangements in the genome of Escherichia coli. Nucleic Acids Res. 2016; 44:7109-19.
141. Finkel S E. Long-term survival during stationary phase: evolution and the GASP phenotype. Nature Reviews Microbiology. 2006; 4:113-20.
142. Zinser E R, Kolter R. Mutations Enhancing Amino Acid Catabolism Confer a Growth Advantage in Stationary Phase. Journal of Bacteriology. 1999; 181:5800-7.
143. Zinser E R, Kolter R. Escherichia coli evolution during stationary phase. Research in Microbiology. 2004; 155:328-36.
144. Zambrano M M, Kolter R. GASPing for Life in Stationary Phase. Cell. 1996; 86:181-4.
145. growth advantage in stationary-phase (GASP) phenomenon in mixed cultures of enterobacteria|FEMS Microbiology Letters Oxford Academic [Internet]. [cited 2019 Mar 10]. Available from: https://academic.oup.com/femsle/article/266/1/119/562771
146. Murrell J C. Physiology of the bacterial cell—A molecular approach: by F. C. Neidhardt, J. L. Ingraham and M. Schaechter, Sinauer Associates, 1990. £34.95 (xii+506 pages) ISBN 0 87893 608 4. Trends in Genetics. 1991; 7:341.
147. Tenaillon O, Barrick J E, Ribeck N, Deatherage D E, Blanchard J L, Dasgupta A, et al. Tempo and mode of genome evolution in a 50,000-generation experiment. Nature. 2016; 536:165-70.
148. Shen Y, Wan Z, Coarfa C, Drabek R, Chen L, Ostrowski E A, et al. A SNP discovery method to assess variant allele probability from next-generation resequencing data. Genome Res. 2010; 20:273-80.
149. Pollutants in urban waste water and sewage sludge. :273.
150. Statovci D, Aguilera M, MacSharry J, Melgar S. The Impact of Western Diet and Nutrients on the Microbiota and Immune Response at Mucosal Interfaces. Front Immunol [Internet]. 2017 [cited 2019 Mar 11];8. Available from: https://www.ncbi.nlm.nih.gov/pmc/articles/PMC5532387/
151. Moore L B, Liu S V, Halliday T M, Neilson A P, Hedrick V E, Davy B M. Urinary Excretion of Sodium, Nitrogen, and Sugar Amounts Are Valid Biomarkers of Dietary Sodium, Protein, and High Sugar Intake in Nonobese Adolescents. J Nutr. 2017; 147:2364-73.
152. Tasevska N. Urinary Sugars—A Biomarker of Total Sugars Intake. Nutrients. 2015; 7:5816-33.
153. Brockman I M, Prather K U. Dynamic knockdown of E. coli central metabolism for redirecting fluxes of primary metabolites. Metabolic Engineering. 2015;28.
154. Wagner C, Urbanczik R. The geometry of the flux cone of a metabolic network. Biophysical journal. 2005; 89:3837-45.
155. Kleijntjens R H. Bioreactors. 2000; 14:329-347.
156. Mannina G, Cosenza A, Di Trapani D, Laudicina V A, Morici C, Odegaard H. Nitrous oxide emissions in a membrane bioreactor treating saline wastewater contaminated by hydrocarbons. Bioresource Technology. 2016; 219:289-97.
157. Zhang Y, Zhu Y, Zhu Y, Li Y. The importance of engineering physiological functionality into microbes.
158. Salazar A N, Gorter de Vries A R, van den Broek M, Wijsman M, de la Torre Cortes P, Brickwedde A, et al. Nanopore sequencing enables near-complete de novo assembly of Saccharomyces cerevisiae reference strain CEN.PK113-7D. FEMS Yeast Research. 2017; 17:1-11.
159. Schmidt M H, Vogel A, Denton A K, Istace B, Wormit A, van de Geest H, et al. De novo Assembly of a New Solanum pennellii Accession Using Nanopore Sequencing. The Plant Cell. 2017;29:tpc.00521.2017.

We claim:

1. A nucleic acid sequence comprising:
a. a first E. coli homology region, wherein the first E. coli homology region comprises a PAM mutation;
b. a constitutive promoter;
c. a mevalonate-3-kinase (M3K) gene comprising a sequence having at least 90% identity to the sequence of SEQ ID NO:1;
d. a mevalonate diphosphate decarboxylase (MVD) gene comprising a sequence having at least 90% identity to the sequence of SEQ ID NO:2; and
e. a second E. coli homology region.

2. The nucleic acid sequence of claim 1, wherein the constitutive promoter is a 16S rRNA promoter.

3. The nucleic acid sequence of claim 1, wherein the constitutive promoter is a T7A1 promoter.

4. The nucleic acid sequence of any one of claims 1-3, wherein the first and second E. coli homology regions are homologous to E. coli strain MG1655.

5. The nucleic acid sequence of any one of claims 1-4, wherein the PAM mutation is CAAA.

6. The nucleic acid sequence of any one of claims 1-5, wherein the MVD gene and the M3K gene are operably linked.

7. The nucleic acid sequence of any one of claims 1-6, wherein the nucleic acid sequence is linear.

8. The nucleic acid sequence of any one of claims 1-7, wherein the M3K gene comprises the sequence of SEQ ID NO:1.

9. The nucleic acid sequence of any one of claims 1-8, wherein the MVD gene comprises the sequence of SEQ ID NO:2.

10. A vector comprising the nucleic acid sequence of any one of claims 1-9.

11. The vector of claim 10, wherein the vector is a viral vector.

12. The vector of claim 10, wherein the vector is a plasmid.

13. A recombinant cell comprising a nucleic acid sequence, wherein the nucleic acid sequence comprises:
a. a first E. coli homology region, wherein the first E. coli homology region comprises a PAM mutation;
b. a constitutive promoter;
c. a mevalonate-3-kinase (M3K) gene comprising a sequence having at least 90% identity to the sequence of SEQ ID NO:1;
d. a mevalonate diphosphate decarboxylase (MVD) gene comprising a sequence having at least 90% identity to the sequence of SEQ ID NO:2; and
e. a second E. coli homology region.

14. The recombinant cell of claim 13, wherein the nucleic acid sequence is integrated into the genome of the recombinant cell.

15. The recombinant cell of any one of claims 13-14, wherein the recombinant cell is a bacterial cell.

16. The recombinant cell of claim 15, wherein the bacterial cell is E. coli.

17. The recombinant cell of any one of claims 14-16, wherein the M3K gene and MVD gene are in a region of the recombinant cell genome known to have higher expression levels.

18. The recombinant cell of claim 17, wherein the region of the genome known to have higher expression levels is the safe site 9 region of E. coli.

19. The recombinant cell of any one of claims 13-17, wherein the M3K gene and the MVD gene are operably linked.

20. The recombinant cell of any one of claims 13-19, wherein the M3K gene and MVD gene are controlled by the constitutive promoter.

21. The recombinant cell of any one of claims 13-20, wherein the constitutive promoter is a 16S rRNA promoter.

22. The recombinant cell of any one of claims 13-20, wherein the constitutive promoter is a T7A1 promoter.

23. The recombinant cell of any one of claims 13-22, wherein the M3K gene comprises the sequence of SEQ ID NO:1.

24. The recombinant cell of any one of claims 13-22, wherein the MVD gene comprises the sequence of SEQ ID NO:2.

25. A method of making a recombinant cell comprising administering the nucleic acid sequence of claim 7 to a cell, wherein the cell incorporates the linear nucleic acid sequence into the cellular genome.

26. The method of claim 25, wherein the recombinant cell is a bacterial cell.

27. The method of any one of claims 25-26, wherein the incorporation of the nucleic acid sequence into the cellular genome occurs through homologous recombination using the first and second E. coli homology regions of the nucleic acid sequence.

28. The method of any one of claims 25-27, further comprising administering a safe site 9 specific gRNA to the recombinant cell.

29. The method of any one of claims 25-28, wherein the recombinant cells comprise Cas9 or a gene encoding Cas9.

30. The method of any one of claims 25-29, wherein only the recombinant cells that incorporate the linear nucleic acid sequence into the cellular genome will remain viable.

31. A method of producing isobutene comprising culturing one or more of the recombinant cells of any one of claims 13-24 under conditions suitable for growth of the recombinant cells, wherein the MVD decarboxylates 3-hydroxyisovalerate (3-HIV) to isobutene, and wherein the M3K catalyzes the phosphorylation of 3-HIV into an unstable 3-phosphate intermediate that undergoes spontaneous decarboxylation to isobutene.

32. The method of claim 31, wherein conditions suitable for growth of the recombinant cells comprises culturing the cells in wasterwater from a water treatment plant.


SEQUENCE LISTING (4 sequences)

SEQ ID NO: 1 - DNA, 978 bp, Artificial Sequence
  synthetic construct; M3K optimized gene
atggagaactataatgttaaaacccgtgcatttccgaccattggtattattctgctgggtggcattagcgacaaaaaaaaccgtattccgctgcataccaccgcaggtattgcatataccggcatcaataacgatgtgtacaccgaaaccaaactgtatgtgagcaaagacgaaaaatcgtatatcgatggcaaagaaatcgatctgaatagcgatcgtagcccgagcaaagtgatcgataaattcaaacatgaaatcctgatgcgtgtgaatctggatgatgaaaacaacctgagcattgatagccgcaattttaacattctgagcggtagcagcgatagcggtgcagcagcactgggtgaatgcattgaaagcatcttcgagtacaacatcaacatcttcacctttgaaaatgatctgcagcgtattagcgaaagcgttggtcgtagcctgtatggtggtctgaccgttaattatgcaaatggtcgtgaaagcctgaccgaaccgctgctggaaccggaagcatttaacaactttaccatcatcggtgcccattttaacattgatcgcaaaccgagcaacgaaatccacgaaaacatcatcaaacatgagaactatcgcgaacgtattaaaagcgcagagcgcaaagcaaaaaaactggaagaactgagccgtaatgccaacattaaaggcatttttgaactggcagaaagcgataccgtggaatatcataaaatgctgcatgatgtgggcgttgatattatcaatgaccgcatggaaaatctgattgaacgcgtgaaagagatgaaaaacaacttctggaacagctatattgttaccggtggtccgaatgtttttgtgatcaccgagaaaaaagatgtggataaagccatggaaggtctgaatgatctgtgtgatgatattcgtctgctgaaagttgcaggtaaaccgcaggttatcagcaaaaacttctaatga

SEQ ID NO: 2 - DNA, 1191 bp, Artificial Sequence
  synthetic construct; MVD optimized gene sequence
atgaccgtttataccgcaagcgttaccgcaccggttaatattgcaaccctgaaatattggggtaaacgtgataccaaactgaatctgccgaccaatagcagcattagcgttaccctgagccaggatgatctgcgtaccctgaccagcgcagcaaccgcaccggaatttgaacgtgataccctgtggctgaatggtgaaccgcatagcattgataatgaacgtacccagaattgtctgcgtgatctgcgtcagctgcgtaaagaaatggaaagcaaagatgcaagcctgccgaccctgagccagtggaaactgcatattgttagcgaaaataattttccgaccgcagcaggtctggcaagcagcgcagcaggttttgcagcactggttagcgcaattgcaaaactgtatcagctgccgcagagcaccagcgaaattagccgtattgcacgtaaaggtagcggtagcgcatgtcgtagcctgtttggtggttatgttgcatgggaaatgggtaaagcagaagatggtcatgatagcatggcagttcagattgcagatagcagcgattggccgcagatgaaagcatgtgttctggttgttagcgatattaaaaaagatgttagcagcacccagggtatgcagctgaccgttgcaaccagcgaactgtttaaagaacgtattgaacatgttgttccgaaacgttttgaagttatgcgtaaagcaattgttgaaaaagatttcgcaacctttgcaaaagaaaccatgatggatagcaatagctttcatgcaacctgtctggatagctttccgccgattttttatatgaatgataccagcaaacgcattattagctggtgtcataccattaatcagttttatggtgaaaccattgtggcatatacctttgatgcaggtccgaatgcagttctgtattatctggcagaaaatgaaagcaaactgtttgcatttatctacaaactgttcggtagcgttccgggttgggataaaaaatttaccaccgaacagctggaagcatttaatcatcagtttgaaagcagcaatttcaccgcacgtgaactggatctggaactgcagaaagatgttgcacgtgttattctgacccaggttggtagcggtccgcaggaaaccaatgaaagcctgattgatgcaaaaaccggtctgccgaaagaataa

SEQ ID NO: 3 - DNA, 975 bp, Picrophilus torridus
atggaaaattacaatgttaagacaagggcgttcccaacaataggcataatactgcttggtgggatctcggataaaaagaacaggataccgctgcatacaacggcaggcatagcatatactggtataaacaatgatgtttacactgagacaaagctttatgtatcaaaagatgaaaaatgctatattgatggaaaggaaattgatttaaattcagatagatcaccatcgaaggttattgataaattcaagcatgaaatacttatgagagtaaatcttgatgatgaaaataacctttcaattgattcaaggaactttaatatattaagtggcagctcagattctggggccgctgcactgggagagtgcatagaatcaatttttgaatacaatataaatatatttacatttgaaaacgatcttcagaggatatcagaaagtgttggaagaagcctttacggtggtttaacagtaaactatgccaatggcagggaatcattaacagagccattacttgagcctgaggcatttaataactttacaataattggtgcacattttaacattgatagaaaaccatcaaatgagattcatgaaaatatcataaaacatgaaaattacagggaaagaataaaaagtgctgagagaaaggcgaaaaaacttgaggagctatcaaggaatgcaaacataaagggtatctttgaacttgcagaatccgatacagtggaataccataaaatgctccatgatgttggcgttgacataataaatgatagaatggagaacctcattgaaagggtaaaagaaatgaaaaataacttctggaattcatacatagttaccggcggcccgaacgtttttgtaataacagagaaaaaggacgttgataaggcaatggaaggattaaatgatctgtgcgatgatataagattattaaaagttgcaggaaagccacaggtcatttcaaaaaacttttaa

SEQ ID NO: 4 - DNA, 1191 bp, Saccharomyces cerevisiae
atgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataa